SEQUENCE LISTING (327 sequences)

SEQ ID NO: 1 - protein, 239 aa, Synthetic construct
  REGION 1..239: note = Description of Artificial Sequence: Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 239 aa, synthetic construct
  REGION 1..239: note = Description of Artificial Sequence: Synthetic polypeptide
MVSKGEELFTGVVPILVELDGDVNGHKFSVSGEGEGDATYGKLTLKFICTTGKLPVPWPTLVTTLTHGVQCFGRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 108 bp, synthetic construct
  misc_feature 1..108: note = Description of Artificial Sequence: Synthetic polynucleotide
  source 1..108: mol_type = other DNA
gacccacggcgtgcagtgcttgttttagagctagaaatagcaagttaaaataaggctagtccgttatcaacttgaaaaagtggcaccgagtcggtgcttttttgtttt

SEQ ID NO: 4 - DNA, 105 bp, synthetic construct
  misc_feature 1..105: note = Description of Artificial Sequence: Synthetic polynucleotide
  source 1..105: mol_type = other DNA
gggtcttcgagaagacctgttttagagctagaaatagcaagttaaaataaggctagtccgttatcaacttgaaaaagtggcaccgagtcggtgcttttttgtttt

SEQ ID NO: 5 - DNA, 107 bp, synthetic construct
  misc_feature 1..107: note = Description of Artificial Sequence: Synthetic polynucleotide
  source 1..107: mol_type = other DNA
ggcccagactgagcacgtgagttttagagctagaaatagcaagttaaaataaggctagtccgttatcaacttgaaaaagtggcaccgagtcggtgcttttttgtttt

SEQ ID NO: 6 - DNA, 107 bp, synthetic construct
  misc_feature 1..107: note = Description of Artificial Sequence: Synthetic polynucleotide
  source 1..107: mol_type = other DNA
gaacacaaagcatagactgcgttttagagctagaaatagcaagttaaaataaggctagtccgttatcaacttgaaaaagtggcaccgagtcggtgcttttttgtttt

SEQ ID NO: 7 - DNA, 107 bp, synthetic construct
  misc_feature 1..107: note = Description of Artificial Sequence: Synthetic polynucleotide
  source 1..107: mol_type = other DNA
ggcactgcggctggaggtgggttttagagctagaaatagcaagttaaaataaggctagtccgttatcaacttgaaaaagtggcaccgagtcggtgcttttttgtttt

SEQ ID NO: 8 - DNA, 107 bp, synthetic construct
  misc_feature 1..107: note = Description of Artificial Sequence: Synthetic polynucleotide
  source 1..107: mol_type = other DNA
gaagcgcctggcagtgtaccgttttagagctagaaatagcaagttaaaataaggctagtccgttatcaacttgaaaaagtggcaccgagtcggtgcttttttgtttt

SEQ ID NO: 9 - DNA, 108 bp, synthetic construct
  misc_feature 1..108: note = Description of Artificial Sequence: Synthetic polynucleotide
  source 1..108: mol_type = other DNA
gtgctgttagcaccctggaaagttttagagctagaaatagcaagttaaaataaggctagtccgttatcaacttgaaaaagtggcaccgagtcggtgcttttttgtttt

SEQ ID NO: 10 - DNA, 107 bp, synthetic construct
  misc_feature 1..107: note = Description of Artificial Sequence: Synthetic polynucleotide
  source 1..107: mol_type = other DNA
gtggcagagcggccagcgctgttttagagctagaaatagcaagttaaaataaggctagtccgttatcaacttgaaaaagtggcaccgagtcggtgcttttttgtttt

SEQ ID NO: 11 - DNA, 108 bp, synthetic construct
  misc_feature 1..108: note = Description of Artificial Sequence: Synthetic polynucleotide
  source 1..108: mol_type = other DNA
gcacccagaccgccaagctgagttttagagctagaaatagcaagttaaaataaggctagtccgttatcaacttgaaaaagtggcaccgagtcggtgcttttttgtttt

SEQ ID NO: 12 - DNA, 107 bp, synthetic construct
  misc_feature 1..107: note = Description of Artificial Sequence: Synthetic polynucleotide
  source 1..107: mol_type = other DNA
gacccaggactcctccctgcgttttagagctagaaatagcaagttaaaataaggctagtccgttatcaacttgaaaaagtggcaccgagtcggtgcttttttgtttt

SEQ ID NO: 13 - DNA, 108 bp, synthetic construct
  misc_feature 1..108: note = Description of Artificial Sequence: Synthetic polynucleotide
  source 1..108: mol_type = other DNA
gcaagcagaggctgaagctgagttttagagctagaaatagcaagttaaaataaggctagtccgttatcaacttgaaaaagtggcaccgagtcggtgcttttttgtttt

SEQ ID NO: 14 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 15 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 16 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 17 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 18 - DNA, 21 bp, synthetic construct
  misc_feature 1..21: note = Description of Artificial Sequence: Synthetic oligonucleotide
  source 1..21: mol_type = other DNA
gacccacggcgtgcagtgctt

SEQ ID NO: 19 - DNA, 21 bp, synthetic construct
  misc_feature 1..21: note = Description of Artificial Sequence: Synthetic oligonucleotide
  source 1..21: mol_type = other DNA
gtgctgttagcaccctggaaa

SEQ ID NO: 20 - DNA, 20 bp, synthetic construct
  misc_feature 1..20: note = Description of Artificial Sequence: Synthetic primer
  source 1..20: mol_type = other DNA
atgtgggctgcctagaaagg

SEQ ID NO: 21 - DNA, 20 bp, synthetic construct
  misc_feature 1..20: note = Description of Artificial Sequence: Synthetic primer
  source 1..20: mol_type = other DNA
cccagccaaacttgtcaacc

SEQ ID NO: 22 - DNA, 20 bp, synthetic construct
  misc_feature 1..20: note = Description of Artificial Sequence: Synthetic primer
  source 1..20: mol_type = other DNA
ccagccccatctgtcaaact

SEQ ID NO: 23 - DNA, 25 bp, synthetic construct
  misc_feature 1..25: note = Description of Artificial Sequence: Synthetic primer
  source 1..25: mol_type = other DNA
tgaatggattccttggaaacaatga

SEQ ID NO: 24 - DNA, 28 bp, synthetic construct
  misc_feature 1..28: note = Description of Artificial Sequence: Synthetic primer
  source 1..28: mol_type = other DNA
tggtcttctttcccctcccctgccctcc

SEQ ID NO: 25 - DNA, 24 bp, synthetic construct
  misc_feature 1..24: note = Description of Artificial Sequence: Synthetic primer
  source 1..24: mol_type = other DNA
ggcctggaggcgggggctcagaga

SEQ ID NO: 26 - DNA, 27 bp, synthetic construct
  misc_feature 1..27: note = Description of Artificial Sequence: Synthetic primer
  source 1..27: mol_type = other DNA
ggacgagaccatgaaggagttgaaggc

SEQ ID NO: 27 - DNA, 25 bp, synthetic construct
  misc_feature 1..25: note = Description of Artificial Sequence: Synthetic primer
  source 1..25: mol_type = other DNA
ccacctgctccttcacctcgtccag

SEQ ID NO: 28 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 29 - DNA, 12 bp, synthetic construct
  misc_feature 1..12: note = Description of Artificial Sequence: Synthetic oligonucleotide
  source 1..12: mol_type = other DNA
ggcctgggtcaa

SEQ ID NO: 30 - DNA, 13 bp, synthetic construct
  misc_feature 1..13: note = Description of Artificial Sequence: Synthetic oligonucleotide
  source 1..13: mol_type = other DNA
ttcctttcctctg

SEQ ID NO: 31 - DNA, 20 bp, synthetic construct
  misc_feature 1..20: note = Description of Artificial Sequence: Synthetic oligonucleotide
  source 1..20: mol_type = other DNA
tcacgtgctcagtctgggcc

SEQ ID NO: 32 - DNA, 19 bp, synthetic construct
  misc_feature 1..19: note = Description of Artificial Sequence: Synthetic oligonucleotide
  source 1..19: mol_type = other DNA
gaggaaaggaagccctgct

SEQ ID NO: 33 - DNA, 15 bp, synthetic construct
  misc_feature 1..15: note = Description of Artificial Sequence: Synthetic oligonucleotide
  source 1..15: mol_type = other DNA
caggccagggctgga

SEQ ID NO: 34 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 35 - DNA, 14 bp, synthetic construct
  misc_feature 1..14: note = Description of Artificial Sequence: Synthetic oligonucleotide
  source 1..14: mol_type = other DNA
cccgctggccctgt

SEQ ID NO: 36 - DNA, 14 bp, synthetic construct
  misc_feature 1..14: note = Description of Artificial Sequence: Synthetic oligonucleotide
  source 1..14: mol_type = other DNA
tcaggctggcccgc

SEQ ID NO: 37 - DNA, 20 bp, synthetic construct
  misc_feature 1..20: note = Description of Artificial Sequence: Synthetic oligonucleotide
  source 1..20: mol_type = other DNA
gcagtctatgctttgtgttc

SEQ ID NO: 38 - DNA, 19 bp, synthetic construct
  misc_feature 1..19: note = Description of Artificial Sequence: Synthetic oligonucleotide
  source 1..19: mol_type = other DNA
ccagcccgctggccctgta

SEQ ID NO: 39 - DNA, 13 bp, synthetic construct
  misc_feature 1..13: note = Description of Artificial Sequence: Synthetic oligonucleotide
  source 1..13: mol_type = other DNA
agctattcaggct

SEQ ID NO: 40 - DNA, 20 bp, synthetic construct
  misc_feature 1..20: note = Description of Artificial Sequence: Synthetic oligonucleotide
  source 1..20: mol_type = other DNA
gtggcactgcggctggaggt

SEQ ID NO: 41 - DNA, 17 bp, synthetic construct
  misc_feature 1..17: note = Description of Artificial Sequence: Synthetic oligonucleotide
  source 1..17: mol_type = other DNA
gatgacaggcaggggca

SEQ ID NO: 42 - DNA, 10 bp, synthetic construct
  misc_feature 1..10: note = Description of Artificial Sequence: Synthetic oligonucleotide
  source 1..10: mol_type = other DNA
cagcaccaga

SEQ ID NO: 43 - DNA, 18 bp, synthetic construct
  misc_feature 1..18: note = Description of Artificial Sequence: Synthetic oligonucleotide
  source 1..18: mol_type = other DNA
acctccagccgcagtgcc

SEQ ID NO: 44 - DNA, 17 bp, synthetic construct
  misc_feature 1..17: note = Description of Artificial Sequence: Synthetic oligonucleotide
  source 1..17: mol_type = other DNA
ccgcggtgcccctgcct

SEQ ID NO: 45 - DNA, 18 bp, synthetic construct
  misc_feature 1..18: note = Description of Artificial Sequence: Synthetic oligonucleotide
  source 1..18: mol_type = other DNA
aagcggagactctggtgc

SEQ ID NO: 46 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 47 - DNA, 14 bp, synthetic construct
  misc_feature 1..14: note = Description of Artificial Sequence: Synthetic oligonucleotide
  source 1..14: mol_type = other DNA
ctgcgcaagctgcg

SEQ ID NO: 48 - DNA, 14 bp, synthetic construct
  misc_feature 1..14: note = Description of Artificial Sequence: Synthetic oligonucleotide
  source 1..14: mol_type = other DNA
tcggcgccctcgcg

SEQ ID NO: 49 - DNA, 20 bp, synthetic construct
  misc_feature 1..20: note = Description of Artificial Sequence: Synthetic oligonucleotide
  source 1..20: mol_type = other DNA
ggtacactgccaggcgcttc

SEQ ID NO: 50 - DNA, 12 bp, synthetic construct
  misc_feature 1..12: note = Description of Artificial Sequence: Synthetic oligonucleotide
  source 1..12: mol_type = other DNA
ggatggcgctga

SEQ ID NO: 51 - DNA, 15 bp, synthetic construct
  misc_feature 1..15: note = Description of Artificial Sequence: Synthetic oligonucleotide
  source 1..15: mol_type = other DNA
gcctcgcctcccacc

SEQ ID NO: 52 - DNA, 19 bp, synthetic construct
  misc_feature 1..19: note = Description of Artificial Sequence: Synthetic primer
  source 1..19: mol_type = other DNA
gatgcgcttccggaagacc

SEQ ID NO: 53 - DNA, 20 bp, synthetic construct
  misc_feature 1..20: note = Description of Artificial Sequence: Synthetic primer
  source 1..20: mol_type = other DNA
gcttcttgagcttctcagcg

SEQ ID NO: 54 - DNA, 21 bp, synthetic construct
  misc_feature 1..21: note = Description of Artificial Sequence: Synthetic primer
  source 1..21: mol_type = other DNA
ggtagcatgttcaggcaccag

SEQ ID NO: 55 - DNA, 27 bp, synthetic construct
  misc_feature 1..27: note = Description of Artificial Sequence: Synthetic primer
  source 1..27: mol_type = other DNA
catccctagtaccgaatcccatatagc

SEQ ID NO: 56 - DNA, 24 bp, synthetic construct
  misc_feature 1..24: note = Description of Artificial Sequence: Synthetic primer
  source 1..24: mol_type = other DNA
catcctcccacctaagcctttcaa

SEQ ID NO: 57 - DNA, 31 bp, synthetic construct
  misc_feature 1..31: note = Description of Artificial Sequence: Synthetic primer
  source 1..31: mol_type = other DNA
ttgagttaatagcattataacaatttccaca

SEQ ID NO: 58 - DNA, 24 bp, synthetic construct
  misc_feature 1..24: note = Description of Artificial Sequence: Synthetic primer
  source 1..24: mol_type = other DNA
actccttacaaccggaaggcaaac

SEQ ID NO: 59 - DNA, 23 bp, synthetic construct
  misc_feature 1..23: note = Description of Artificial Sequence: Synthetic primer
  source 1..23: mol_type = other DNA
tggacgtggtgaagcccgtggtg

SEQ ID NO: 60 - DNA, 21 bp, synthetic construct
  misc_feature 1..21: note = Description of Artificial Sequence: Synthetic primer
  source 1..21: mol_type = other DNA
taggtctctagggggcctctg

SEQ ID NO: 61 - DNA, 21 bp, synthetic construct
  misc_feature 1..21: note = Description of Artificial Sequence: Synthetic primer
  source 1..21: mol_type = other DNA
aggctgcccaacagccccact

SEQ ID NO: 62 - DNA, 20 bp, synthetic construct
  misc_feature 1..20: note = Description of Artificial Sequence: Synthetic primer
  source 1..20: mol_type = other DNA
tcccctgttgacctggagaa

SEQ ID NO: 63 - DNA, 20 bp, synthetic construct
  misc_feature 1..20: note = Description of Artificial Sequence: Synthetic primer
  source 1..20: mol_type = other DNA
cactgtacttgccctgacca

SEQ ID NO: 64 - DNA, 19 bp, synthetic construct
  misc_feature 1..19: note = Description of Artificial Sequence: Synthetic primer
  source 1..19: mol_type = other DNA
tgagatgtgggcagaaggg

SEQ ID NO: 65 - DNA, 20 bp, synthetic construct
  misc_feature 1..20: note = Description of Artificial Sequence: Synthetic primer
  source 1..20: mol_type = other DNA
ttggtgttgacagggagcaa

SEQ ID NO: 66 - DNA, 20 bp, synthetic construct
  misc_feature 1..20: note = Description of Artificial Sequence: Synthetic primer
  source 1..20: mol_type = other DNA
gtccaaaggcccaagaacct

SEQ ID NO: 67 - DNA, 20 bp, synthetic construct
  misc_feature 1..20: note = Description of Artificial Sequence: Synthetic primer
  source 1..20: mol_type = other DNA
tgagagggaacagaagggct

SEQ ID NO: 68 - DNA, 22 bp, synthetic construct
  misc_feature 1..22: note = Description of Artificial Sequence: Synthetic primer
  source 1..22: mol_type = other DNA
gctcatcttaatctgctcagcc

SEQ ID NO: 69 - DNA, 21 bp, synthetic construct
  misc_feature 1..21: note = Description of Artificial Sequence: Synthetic primer
  source 1..21: mol_type = other DNA
tcctagcactttggaaggtcg

SEQ ID NO: 70 - DNA, 20 bp, synthetic construct
  misc_feature 1..20: note = Description of Artificial Sequence: Synthetic primer
  source 1..20: mol_type = other DNA
aaaggagcagctcttcctgg

SEQ ID NO: 71 - DNA, 20 bp, synthetic construct
  misc_feature 1..20: note = Description of Artificial Sequence: Synthetic primer
  source 1..20: mol_type = other DNA
gtctgcaccatctcccacaa

SEQ ID NO: 72 - DNA, 21 bp, synthetic construct
  misc_feature 1..21: note = Description of Artificial Sequence: Synthetic primer
  source 1..21: mol_type = other DNA
gtgtggagagtgagtaagcca

SEQ ID NO: 73 - DNA, 21 bp, synthetic construct
  misc_feature 1..21: note = Description of Artificial Sequence: Synthetic primer
  source 1..21: mol_type = other DNA
acggtaggatgatttcaggca

SEQ ID NO: 74 - DNA, 26 bp, synthetic construct
  misc_feature 1..26: note = Description of Artificial Sequence: Synthetic primer
  source 1..26: mol_type = other DNA
ttttttggtactcgagtgttattcag

SEQ ID NO: 75 - DNA, 21 bp, synthetic construct
  misc_feature 1..21: note = Description of Artificial Sequence: Synthetic primer
  source 1..21: mol_type = other DNA
cacaaagcagtgtagctcagg

SEQ ID NO: 76 - DNA, 20 bp, synthetic construct
  misc_feature 1..20: note = Description of Artificial Sequence: Synthetic primer
  source 1..20: mol_type = other DNA
ggcatggcttctgagactca

SEQ ID NO: 77 - DNA, 21 bp, synthetic construct
  misc_feature 1..21: note = Description of Artificial Sequence: Synthetic primer
  source 1..21: mol_type = other DNA
ccccttgcactccctgtcttt

SEQ ID NO: 78 - DNA, 20 bp, synthetic construct
  misc_feature 1..20: note = Description of Artificial Sequence: Synthetic primer
  source 1..20: mol_type = other DNA
gaagaggctgcccatgagag

SEQ ID NO: 79 - DNA, 20 bp, synthetic construct
  misc_feature 1..20: note = Description of Artificial Sequence: Synthetic primer
  source 1..20: mol_type = other DNA
tttggcaatggaggcattgg

SEQ ID NO: 80 - DNA, 20 bp, synthetic construct
  misc_feature 1..20: note = Description of Artificial Sequence: Synthetic primer
  source 1..20: mol_type = other DNA
ggtctgaggctcgaatcctg

SEQ ID NO: 81 - DNA, 20 bp, synthetic construct
  misc_feature 1..20: note = Description of Artificial Sequence: Synthetic primer
  source 1..20: mol_type = other DNA
ctgtggcctccatatccctg

SEQ ID NO: 82 - DNA, 20 bp, synthetic construct
  misc_feature 1..20: note = Description of Artificial Sequence: Synthetic primer
  source 1..20: mol_type = other DNA
tttccaccagaactcagccc

SEQ ID NO: 83 - DNA, 20 bp, synthetic construct
  misc_feature 1..20: note = Description of Artificial Sequence: Synthetic primer
  source 1..20: mol_type = other DNA
cctcggttcctccacaacac

SEQ ID NO: 84 - DNA, 20 bp, synthetic construct
  misc_feature 1..20: note = Description of Artificial Sequence: Synthetic primer
  source 1..20: mol_type = other DNA
gcaggggagggataaagcag

SEQ ID NO: 85 - DNA, 20 bp, synthetic construct
  misc_feature 1..20: note = Description of Artificial Sequence: Synthetic primer
  source 1..20: mol_type = other DNA
cacgggaaggacaggagaag

SEQ ID NO: 86 - DNA, 20 bp, synthetic construct
  misc_feature 1..20: note = Description of Artificial Sequence: Synthetic primer
  source 1..20: mol_type = other DNA
atgtgggctgcctagaaagg

SEQ ID NO: 87 - DNA, 20 bp, synthetic construct
  misc_feature 1..20: note = Description of Artificial Sequence: Synthetic primer
  source 1..20: mol_type = other DNA
cccagccaaacttgtcaacc

SEQ ID NO: 88 - DNA, 27 bp, synthetic construct
  misc_feature 1..27: note = Description of Artificial Sequence: Synthetic primer
  source 1..27: mol_type = other DNA
ggacgagaccatgaaggagttgaaggc

SEQ ID NO: 89 - DNA, 25 bp, synthetic construct
  misc_feature 1..25: note = Description of Artificial Sequence: Synthetic primer
  source 1..25: mol_type = other DNA
ccacctgctccttcacctcgtccag

SEQ ID NO: 90 - DNA, 21 bp, synthetic construct
  misc_feature 1..21: note = Description of Artificial Sequence: Synthetic oligonucleotide
  source 1..21: mol_type = other DNA
gacccacggcgtgcagtgctt

SEQ ID NO: 91 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 92 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 93 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 94 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 95 - DNA, 20 bp, synthetic construct
  misc_feature 1..20: note = Description of Artificial Sequence: Synthetic oligonucleotide
  source 1..20: mol_type = other DNA
gtggcagagcggccagcgct

SEQ ID NO: 96 - DNA, 21 bp, synthetic construct
  misc_feature 1..21: note = Description of Artificial Sequence: Synthetic oligonucleotide
  source 1..21: mol_type = other DNA
gcacccagaccgccaagctga

SEQ ID NO: 97 - DNA, 20 bp, synthetic construct
  misc_feature 1..20: note = Description of Artificial Sequence: Synthetic oligonucleotide
  source 1..20: mol_type = other DNA
gacccaggactcctccctgc

SEQ ID NO: 98 - DNA, 21 bp, synthetic construct
  misc_feature 1..21: note = Description of Artificial Sequence: Synthetic oligonucleotide
  source 1..21: mol_type = other DNA
gcaagcagaggctgaagctga

SEQ ID NO: 99 - DNA, 18 bp, synthetic construct
  misc_feature 1..18: note = Description of Artificial Sequence: Synthetic oligonucleotide
  source 1..18: mol_type = other DNA
gggtcttcgagaagacct

SEQ ID NO: 100 - DNA, 20 bp, synthetic construct
  misc_feature 1..20: note = Description of Artificial Sequence: Synthetic primer
  source 1..20: mol_type = other DNA
atgtgggctgcctagaaagg

SEQ ID NO: 101 - DNA, 20 bp, synthetic construct
  misc_feature 1..20: note = Description of Artificial Sequence: Synthetic primer
  source 1..20: mol_type = other DNA
cccagccaaacttgtcaacc

SEQ ID NO: 102 - DNA, 20 bp, synthetic construct
  misc_feature 1..20: note = Description of Artificial Sequence: Synthetic primer
  source 1..20: mol_type = other DNA
ccagccccatctgtcaaact

SEQ ID NO: 103 - DNA, 25 bp, synthetic construct
  misc_feature 1..25: note = Description of Artificial Sequence: Synthetic primer
  source 1..25: mol_type = other DNA
tgaatggattccttggaaacaatga

SEQ ID NO: 104 - DNA, 28 bp, synthetic construct
  misc_feature 1..28: note = Description of Artificial Sequence: Synthetic primer
  source 1..28: mol_type = other DNA
tggtcttctttcccctcccctgccctcc

SEQ ID NO: 105 - DNA, 24 bp, synthetic construct
  misc_feature 1..24: note = Description of Artificial Sequence: Synthetic primer
  source 1..24: mol_type = other DNA
ggcctggaggcgggggctcagaga

SEQ ID NO: 106 - DNA, 27 bp, synthetic construct
  misc_feature 1..27: note = Description of Artificial Sequence: Synthetic primer
  source 1..27: mol_type = other DNA
ggacgagaccatgaaggagttgaaggc

SEQ ID NO: 107 - DNA, 25 bp, synthetic construct
  misc_feature 1..25: note = Description of Artificial Sequence: Synthetic primer
  source 1..25: mol_type = other DNA
ccacctgctccttcacctcgtccag

SEQ ID NO: 108 - DNA, 22 bp, synthetic construct
  misc_feature 1..22: note = Description of Artificial Sequence: Synthetic primer
  source 1..22: mol_type = other DNA
tcagaaggaccctgacccacct

SEQ ID NO: 109 - DNA, 26 bp, synthetic construct
  misc_feature 1..26: note = Description of Artificial Sequence: Synthetic primer
  source 1..26: mol_type = other DNA
atgaaacctggacctggggaggtata

SEQ ID NO: 110 - DNA, 20 bp, synthetic construct
  misc_feature 1..20: note = Description of Artificial Sequence: Synthetic primer
  source 1..20: mol_type = other DNA
agctgtgaccggcgcctacg

SEQ ID NO: 111 - DNA, 20 bp, synthetic construct
  misc_feature 1..20: note = Description of Artificial Sequence: Synthetic primer
  source 1..20: mol_type = other DNA
gggattctcctccacgtcac

SEQ ID NO: 112 - DNA, 19 bp, synthetic construct
  misc_feature 1..19: note = Description of Artificial Sequence: Synthetic primer
  source 1..19: mol_type = other DNA
gatgcgcttccggaagacc

SEQ ID NO: 113 - DNA, 20 bp, synthetic construct
  misc_feature 1..20: note = Description of Artificial Sequence: Synthetic primer
  source 1..20: mol_type = other DNA
gcttcttgagcttctcagcg

SEQ ID NO: 114 - DNA, 21 bp, synthetic construct
  misc_feature 1..21: note = Description of Artificial Sequence: Synthetic primer
  source 1..21: mol_type = other DNA
ggtagcatgttcaggcaccag

SEQ ID NO: 115 - DNA, 27 bp, synthetic construct
  misc_feature 1..27: note = Description of Artificial Sequence: Synthetic primer
  source 1..27: mol_type = other DNA
catccctagtaccgaatcccatatagc

SEQ ID NO: 116 - DNA, 24 bp, synthetic construct
  misc_feature 1..24: note = Description of Artificial Sequence: Synthetic primer
  source 1..24: mol_type = other DNA
catcctcccacctaagcctttcaa

SEQ ID NO: 117 - DNA, 31 bp, synthetic construct; identical to SEQ ID NO: 57
  misc_feature 1..31: note = Description of Artificial Sequence: Synthetic primer
  source 1..31: mol_type = other DNA

SEQ ID NO: 118 - DNA, 25 bp, synthetic construct
  misc_feature 1..25: note = Description of Artificial Sequence: Synthetic primer
  source 1..25: mol_type = other DNA
gatacaccataaaggggtttgactg

SEQ ID NO: 119 - DNA, 23 bp, synthetic construct
  misc_feature 1..23: note = Description of Artificial Sequence: Synthetic primer
  source 1..23: mol_type = other DNA
accatttccccccaattctactc

SEQ ID NO: 120 - DNA, 23 bp, synthetic construct
  misc_feature 1..23: note = Description of Artificial Sequence: Synthetic primer
  source 1..23: mol_type = other DNA
catctgcattggcttgaaacatc

SEQ ID NO: 121 - DNA, 26 bp, synthetic construct
  misc_feature 1..26: note = Description of Artificial Sequence: Synthetic primer
  source 1..26: mol_type = other DNA
ttacaaaagtgctaaatgatgcacat

SEQ ID NO: 122 - DNA, 23 bp, synthetic construct
  misc_feature 1..23: note = Description of Artificial Sequence: Synthetic primer
  source 1..23: mol_type = other DNA
actcagtaaagctcctcttcaac

SEQ ID NO: 123 - DNA, 21 bp, synthetic construct
  misc_feature 1..21: note = Description of Artificial Sequence: Synthetic primer
  source 1..21: mol_type = other DNA
ttttgcttaggtccactgggc

SEQ ID NO: 124 - DNA, 20 bp, synthetic construct
  misc_feature 1..20: note = Description of Artificial Sequence: Synthetic oligonucleotide
  source 1..20: mol_type = other DNA
gttgatggggtggttcagga

SEQ ID NO: 125 - DNA, 20 bp, synthetic construct
  misc_feature 1..20: note = Description of Artificial Sequence: Synthetic oligonucleotide
  source 1..20: mol_type = other DNA
gttgatgaggtggttcagga

SEQ ID NO: 126 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 127 - DNA, 20 bp, synthetic construct
  misc_feature 1..20: note = Description of Artificial Sequence: Synthetic oligonucleotide
  source 1..20: mol_type = other DNA
gagtatgaggcatagactgc

SEQ ID NO: 128 - DNA, 20 bp, synthetic construct
  misc_feature 1..20: note = Description of Artificial Sequence: Synthetic oligonucleotide
  source 1..20: mol_type = other DNA
gatgagataatgatgagtca

SEQ ID NO: 129 - DNA, 20 bp, synthetic construct
  misc_feature 1..20: note = Description of Artificial Sequence: Synthetic oligonucleotide
  source 1..20: mol_type = other DNA
ggattgacccaggccagggc

SEQ ID NO: 130 - DNA, 20 bp, synthetic construct
  misc_feature 1..20: note = Description of Artificial Sequence: Synthetic oligonucleotide
  source 1..20: mol_type = other DNA
gtagaaaaagtatagactgc

SEQ ID NO: 131 - DNA, 21 bp, synthetic construct
  misc_feature 1..21: note = Description of Artificial Sequence: Synthetic oligonucleotide
  source 1..21: mol_type = other DNA
gcttgaccaatagccttgaca

SEQ ID NO: 132 - DNA, 21 bp, synthetic construct
  misc_feature 1..21: note = Description of Artificial Sequence: Synthetic oligonucleotide
  source 1..21: mol_type = other DNA
gatatttgcattgagatagtg

SEQ ID NO: 133 - DNA, 20 bp, synthetic construct
  misc_feature 1..20: note = Description of Artificial Sequence: Synthetic oligonucleotide
  source 1..20: mol_type = other DNA
gaaaatagttgaagaaaaag

SEQ ID NO: 134 - DNA, 21 bp, synthetic construct
  misc_feature 1..21: note = Description of Artificial Sequence: Synthetic oligonucleotide
  source 1..21: mol_type = other DNA
gcacagaagataccgagactg

SEQ ID NO: 135 - DNA, 18 bp, synthetic construct
  misc_feature 1..18: note = Description of Artificial Sequence: Synthetic oligonucleotide
  source 1..18: mol_type = other DNA
gggtcttcgagaagacct

SEQ ID NO: 136 - DNA, 25 bp, synthetic construct
  misc_feature 1..25: note = Description of Artificial Sequence: Synthetic primer
  source 1..25: mol_type = other DNA
tccttggaaacaatgataacaagac

SEQ ID NO: 137 - DNA, 20 bp, synthetic construct
  misc_feature 1..20: note = Description of Artificial Sequence: Synthetic primer
  source 1..20: mol_type = other DNA
ccagccccatctgtcaaact

SEQ ID NO: 138 - DNA, 25 bp, synthetic construct
  misc_feature 1..25: note = Description of Artificial Sequence: Synthetic primer
  source 1..25: mol_type = other DNA
gcttatattctagggagacagacat

SEQ ID NO: 139 - DNA, 22 bp, synthetic construct
  misc_feature 1..22: note = Description of Artificial Sequence: Synthetic primer
  source 1..22: mol_type = other DNA
acctgaggtcagaagtttgaga

SEQ ID NO: 140 - DNA, 20 bp, synthetic construct
  misc_feature 1..20: note = Description of Artificial Sequence: Synthetic primer
  source 1..20: mol_type = other DNA
gtctgaggtcacacagtggg

SEQ ID NO: 141 - DNA, 20 bp, synthetic construct
  misc_feature 1..20: note = Description of Artificial Sequence: Synthetic primer
  source 1..20: mol_type = other DNA
agagcagggaccacatctac

SEQ ID NO: 142 - DNA, 20 bp, synthetic construct
  misc_feature 1..20: note = Description of Artificial Sequence: Synthetic primer
  source 1..20: mol_type = other DNA
gccaaacttgtcaaccagta

SEQ ID NO: 143 - DNA, 20 bp, synthetic construct
  misc_feature 1..20: note = Description of Artificial Sequence: Synthetic primer
  source 1..20: mol_type = other DNA
atgtgggctgcctagaaagg

SEQ ID NO: 144 - DNA, 33 bp, synthetic construct
  misc_feature 1..33: note = Description of Artificial Sequence: Synthetic primer
  source 1..33: mol_type = other DNA
tccatttatatgaaatgttcagaaaaggcaaat

SEQ ID NO: 145 - DNA, 32 bp, synthetic construct
  misc_feature 1..32: note = Description of Artificial Sequence: Synthetic primer
  source 1..32: mol_type = other DNA
gtaactatatgctctctgattctcctattagc

SEQ ID NO: 146 - DNA, 18 bp, synthetic construct
  misc_feature 1..18: note = Description of Artificial Sequence: Synthetic primer
  source 1..18: mol_type = other DNA
cctaccttcccagggttt

SEQ ID NO: 147 - DNA, 23 bp, synthetic construct
  misc_feature 1..23: note = Description of Artificial Sequence: Synthetic primer
  source 1..23: mol_type = other DNA
aagaagtcctggtatcttctatg

SEQ ID NO: 148 - DNA, 20 bp, synthetic construct
  misc_feature 1..20: note = Description of Artificial Sequence: Synthetic primer
  source 1..20: mol_type = other DNA
tcagacgttccagaagcgag

SEQ ID NO: 149 - DNA, 23 bp, synthetic construct
  misc_feature 1..23: note = Description of Artificial Sequence: Synthetic primer
  source 1..23: mol_type = other DNA
gacaagaaggtgaaaaacggctg

SEQ ID NO: 150 - DNA, 25 bp, synthetic construct
  misc_feature 1..25: note = Description of Artificial Sequence: Synthetic primer
  source 1..25: mol_type = other DNA
gattcaaagcataccagagaatagt

SEQ ID NO: 151 - DNA, 25 bp, synthetic construct
  misc_feature 1..25: note = Description of Artificial Sequence: Synthetic primer
  source 1..25: mol_type = other DNA
tcaaactagtatgcatttcaacaac

SEQ ID NO: 152 - DNA, 24 bp, synthetic construct
  misc_feature 1..24: note = Description of Artificial Sequence: Synthetic primer
  source 1..24: mol_type = other DNA
gagtgtagctgtttttctcaggtt

SEQ ID NO: 153 - DNA, 25 bp, synthetic construct
  misc_feature 1..25: note = Description of Artificial Sequence: Synthetic primer
  source 1..25: mol_type = other DNA
gaatacccaaccataagaagaacag

SEQ ID NO: 154 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 155 - DNA, 20 bp, synthetic construct
  misc_feature 1..20: note = Description of Artificial Sequence: Synthetic oligonucleotide
  source 1..20: mol_type = other DNA
gagtatgaggcatagactgc

SEQ ID NO: 156 - DNA, 20 bp, synthetic construct
  misc_feature 1..20: note = Description of Artificial Sequence: Synthetic oligonucleotide
  source 1..20: mol_type = other DNA
gatgagataatgatgagtca

SEQ ID NO: 157 - DNA, 20 bp, synthetic construct
  misc_feature 1..20: note = Description of Artificial Sequence: Synthetic oligonucleotide
  source 1..20: mol_type = other DNA
ggattgacccaggccagggc

SEQ ID NO: 158 - DNA, 20 bp, synthetic construct
  misc_feature 1..20: note = Description of Artificial Sequence: Synthetic oligonucleotide
  source 1..20: mol_type = other DNA
gcagtctatactttttctac

SEQ ID NO: 159 - DNA, 20 bp, synthetic construct
  misc_feature 1..20: note = Description of Artificial Sequence: Synthetic oligonucleotide
  source 1..20: mol_type = other DNA
cttgaccaatagccttgaca

SEQ ID NO: 160 - DNA, 20 bp, synthetic construct
  misc_feature 1..20: note = Description of Artificial Sequence: Synthetic oligonucleotide
  source 1..20: mol_type = other DNA
atatttgcattgagatagtg

SEQ ID NO: 161 - DNA, 20 bp, synthetic construct
  misc_feature 1..20: note = Description of Artificial Sequence: Synthetic oligonucleotide
  source 1..20: mol_type = other DNA
gaaaatagttgaagaaaaag

SEQ ID NO: 162 - DNA, 20 bp, synthetic construct
  misc_feature 1..20: note = Description of Artificial Sequence: Synthetic oligonucleotide
  source 1..20: mol_type = other DNA
cacagaagataccgagactg

SEQ ID NO: 163 - DNA, 22 bp, synthetic construct
  misc_feature 1..22: note = Description of Artificial Sequence: Synthetic primer
  source 1..22: mol_type = other DNA
cagcattatccatttgctgcca

SEQ ID NO: 164 - DNA, 21 bp, synthetic construct
  misc_feature 1..21: note = Description of Artificial Sequence: Synthetic primer
  source 1..21: mol_type = other DNA
tggagacagcgagtctacagc

SEQ ID NO: 165 - DNA, 25 bp, synthetic construct
  misc_feature 1..25: note = Description of Artificial Sequence: Synthetic primer
  source 1..25: mol_type = other DNA
taacaccattatagctgaagtgggg

SEQ ID NO: 166 - DNA, 27 bp, synthetic construct
  misc_feature 1..27: note = Description of Artificial Sequence: Synthetic primer
  source 1..27: mol_type = other DNA
tgagttacacacaagccagttaaattc

SEQ ID NO: 167 - DNA, 20 bp, synthetic construct
  misc_feature 1..20: note = Description of Artificial Sequence: Synthetic primer
  source 1..20: mol_type = other DNA
agggagtggacatgaggcga

SEQ ID NO: 168 - DNA, 20 bp, synthetic construct
  misc_feature 1..20: note = Description of Artificial Sequence: Synthetic primer
  source 1..20: mol_type = other DNA
cccaagaggaagtcccaagg

SEQ ID NO: 169 - DNA, 20 bp, synthetic construct
  misc_feature 1..20: note = Description of Artificial Sequence: Synthetic primer
  source 1..20: mol_type = other DNA
ccttgggaagagaaggggtc

SEQ ID NO: 170 - DNA, 26 bp, synthetic construct
  misc_feature 1..26: note = Description of Artificial Sequence: Synthetic primer
  source 1..26: mol_type = other DNA
gagataccggaagctttgatgtaaga

SEQ ID NO: 171 - DNA, 20 bp, synthetic construct
  misc_feature 1..20: note = Description of Artificial Sequence: Synthetic primer
  source 1..20: mol_type = other DNA
cttggggagaaaggtccagg

SEQ ID NO: 172 - DNA, 26 bp, synthetic construct
  misc_feature 1..26: note = Description of Artificial Sequence: Synthetic primer
  source 1..26: mol_type = other DNA
caagcttttcctcctgggatgtaaaa

SEQ ID NO: 173 - DNA, 21 bp, synthetic construct
  misc_feature 1..21: note = Description of Artificial Sequence: Synthetic primer
  source 1..21: mol_type = other DNA
ctggcaagctgttctcacatg

SEQ ID NO: 174 - DNA, 20 bp, synthetic construct
  misc_feature 1..20: note = Description of Artificial Sequence: Synthetic primer
  source 1..20: mol_type = other DNA
gaggctgaggcaggagtatg

SEQ ID NO: 175 - DNA, 31 bp, synthetic construct
  misc_feature 1..31: note = Description of Artificial Sequence: Synthetic primer
  source 1..31: mol_type = other DNA
gttttcagtagaagagtatataatacataat

SEQ ID NO: 176 - DNA, 20 bp, synthetic construct
  misc_feature 1..20: note = Description of Artificial Sequence: Synthetic primer
  source 1..20: mol_type = other DNA
atattctcagcctaggcctg

SEQ ID NO: 177 - DNA, 22 bp, synthetic construct
  misc_feature 1..22: note = Description of Artificial Sequence: Synthetic primer
  source 1..22: mol_type = other DNA
tgttggacatgggtgccttatt

SEQ ID NO: 178 - DNA, 20 bp, synthetic construct
  misc_feature 1..20: note = Description of Artificial Sequence: Synthetic primer
  source 1..20: mol_type = other DNA
ttcaccctctctggatggcg

SEQ ID NO: 179 - DNA, 20 bp, synthetic construct
  misc_feature 1..20: note = Description of Artificial Sequence: Synthetic primer
  source 1..20: mol_type = other DNA
gcaggaggaggcagtgaaag

SEQ ID NO: 180 - DNA, 25 bp, synthetic construct
  misc_feature 1..25: note = Description of Artificial Sequence: Synthetic primer
  source 1..25: mol_type = other DNA
cagagaaataacactctggcagctg

SEQ ID NO: 181 - DNA, 28 bp, synthetic construct
  misc_feature 1..28: note = Description of Artificial Sequence: Synthetic primer
  source 1..28: mol_type = other DNA
cagcatttatcacgcagtattgttattg

SEQ ID NO: 182 - DNA, 30 bp, synthetic construct
  misc_feature 1..30: note = Description of Artificial Sequence: Synthetic primer
  source 1..30: mol_type = other DNA
tcatttcgtgttgtgctttatcacttaaaa

SEQ ID NO: 183 - DNA, 35 bp, synthetic construct
  misc_feature 1..35: note = Description of Artificial Sequence: Synthetic primer
  source 1..35: mol_type = other DNA
gtgagcagtaaacttaattgttgatacaataaatc

SEQ ID NO: 184 - DNA, 35 bp, synthetic construct
  misc_feature 1..35: note = Description of Artificial Sequence: Synthetic primer
  source 1..35: mol_type = other DNA
cttttagaatgaaagtgtgcatcttagtaaagaaa

SEQ ID NO: 185 - DNA, 23 bp, synthetic construct
  misc_feature 1..23: note = Description of Artificial Sequence: Synthetic primer
  source 1..23: mol_type = other DNA
gttcctcactgattctcagcagg

SEQ ID NO: 186 - DNA, 29 bp, synthetic construct
  misc_feature 1..29: note = Description of Artificial Sequence: Synthetic primer
  source 1..29: mol_type = other DNA
cacaaaagggataaatgctctatccattt

SEQ ID NO: 187 - DNA, 3439 bp, synthetic construct
  misc_feature 1..3439: note = Description of Artificial Sequence: Synthetic polynucleotide
  source 1..3439: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgatggggtggttcaggagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgctttttttctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 188 - DNA, 3439 bp, synthetic construct
  misc_feature 1..3439: note = Description of Artificial Sequence: Synthetic polynucleotide
  source 1..3439: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgatgaggtggttcaggagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgctttttttctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 189 - DNA, 5849 bp, synthetic construct
  misc_feature 1..5849: note = Description of Artificial Sequence: Synthetic polynucleotide
  source 1..5849: mol_type = other DNA
gtcgacattgattattgactagatcatcgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcctacgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagccccgggagggcagaggaagtcttctaacatgcggtgacgtggaggagaatcccggccctactagttgatggggtggttcaggaggggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 190 - DNA, 5849 bp, synthetic construct
  misc_feature 1..5849: note = Description of Artificial Sequence: Synthetic polynucleotide
  source 1..5849: mol_type = other DNA
gtcgacattgattattgactagatcatcgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcctacgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagccccgggagggcagaggaagtcttctaacatgcggtgacgtggaggagaatcccggccctactagttgatgaggtggttcaggaggggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 191 - DNA, 9319 bp, synthetic construct
  misc_feature 1..9319: note = Description of Artificial Sequence: Synthetic polynucleotide
  source 1..9319: mol_type = other DNA
gctgcttcgcgatgtacgggccagatatacgcgttgacattgattattgactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggcggccgctaatacgactcactatagggagagccgccaccatgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgc

SEQ ID NO: 192 - DNA, 9403 bp, synthetic construct
  misc_feature 1..9403: note = Description of Artificial Sequence: Synthetic polynucleotide
  source 1..9403: mol_type = other DNA
gctgcttcgcgatgtacgggccagatatacgcgttgacattgattattgactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggcggccgctaatacgactcactatagggagagccgccaccatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgc

SEQ ID NO: 193 - protein, 10 aa, synthetic construct
  REGION 1..10: note = Description of Artificial Sequence: Synthetic peptide
LTHGVQCFGR

SEQ ID NO: 194 - DNA, 30 bp, synthetic construct
  misc_feature 1..30: note = Description of Artificial Sequence: Synthetic oligonucleotide
  source 1..30: mol_type = other DNA
ctgacccacggcgtgcagtgcttcggccgc

SEQ ID NO: 195 - protein, 10 aa, synthetic construct
  REGION 1..10: note = Description of Artificial Sequence: Synthetic peptide
LTYGVQCFGR

SEQ ID NO: 196 - DNA, 30 bp, synthetic construct
  misc_feature 1..30: note = Description of Artificial Sequence: Synthetic oligonucleotide
  source 1..30: mol_type = other DNA
ctgacctacggcgtgcagtgcttcggccgc

SEQ ID NO: 197 - DNA, 29 bp, synthetic construct
  misc_feature 1..29: note = Description of Artificial Sequence: Synthetic oligonucleotide
  source 1..29: mol_type = other DNA
cctgacccacggcgtgcagtgcttcggcc

SEQ ID NO: 198 - DNA, 36 bp, synthetic construct
  misc_feature 1..36: note = Description of Artificial Sequence: Synthetic oligonucleotide
  source 1..36: mol_type = other DNA
cgaaacaccgggtcttcgagaagacctgttttagag

SEQ ID NO: 199 - DNA, 36 bp, synthetic construct
  misc_feature 1..36: note = Description of Artificial Sequence: Synthetic oligonucleotide
  source 1..36: mol_type = other DNA
ctctaaaacaggtcttctcgaagacccggtgtttcg

SEQ ID NO: 200 - DNA, 25 bp, synthetic construct
  misc_feature 1..25: note = Description of Artificial Sequence: Synthetic oligonucleotide
  source 1..25: mol_type = other DNA
agttgatggggtggttcaggagggg

SEQ ID NO: 201 - DNA, 25 bp, synthetic construct
  misc_feature 1..25: note = Description of Artificial Sequence: Synthetic oligonucleotide
  source 1..25: mol_type = other DNA
agttgatgaggtggttcaggagggg

SEQ ID NO: 202 - DNA, 25 bp, synthetic construct
  misc_feature 1..25: note = Description of Artificial Sequence: Synthetic oligonucleotide
  source 1..25: mol_type = other DNA
agttggtggggtggttcaggagggg

SEQ ID NO: 203 - DNA, 25 bp, synthetic construct
  misc_feature 1..25: note = Description of Artificial Sequence: Synthetic oligonucleotide
  source 1..25: mol_type = other DNA
agttggtggggtggttcaggagggg

SEQ ID NO: 204 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 205 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 206 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 207 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 208 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 209 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 210 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 211 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 212 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 213 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 214 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 215 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 216 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 217 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 218 - DNA, 20 bp, synthetic construct
  misc_feature 1..20: note = Description of Artificial Sequence: Synthetic oligonucleotide
  source 1..20: mol_type = other DNA
ccctacatcgtgcagtgctt

SEQ ID NO: 219 - DNA, 20 bp, synthetic construct
  misc_feature 1..20: note = Description of Artificial Sequence: Synthetic oligonucleotide
  source 1..20: mol_type = other DNA
ccccaagtagtgcagtgctt

SEQ ID NO: 220 - DNA, 20 bp, synthetic construct
  misc_feature 1..20: note = Description of Artificial Sequence: Synthetic oligonucleotide
  source 1..20: mol_type = other DNA
aaccaagatgtgcagtgctt

SEQ ID NO: 221 - DNA, 20 bp, synthetic construct
  misc_feature 1..20: note = Description of Artificial Sequence: Synthetic oligonucleotide
  source 1..20: mol_type = other DNA
aaccagcgcctgcagtgctt

SEQ ID NO: 222 - DNA, 20 bp, synthetic construct
  misc_feature 1..20: note = Description of Artificial Sequence: Synthetic oligonucleotide
  source 1..20: mol_type = other DNA
ccccatggcttgctgtgctt

SEQ ID NO: 223 - DNA, 20 bp, synthetic construct
  misc_feature 1..20: note = Description of Artificial Sequence: Synthetic oligonucleotide
  source 1..20: mol_type = other DNA
cacccagactgagcacgtgc

SEQ ID NO: 224 - DNA, 20 bp, synthetic construct
  misc_feature 1..20: note = Description of Artificial Sequence: Synthetic oligonucleotide
  source 1..20: mol_type = other DNA
gacacagaccgggcacgtga

SEQ ID NO: 225 - DNA, 20 bp, synthetic construct
  misc_feature 1..20: note = Description of Artificial Sequence: Synthetic oligonucleotide
  source 1..20: mol_type = other DNA
agctcagactgagcaagtga

SEQ ID NO: 226 - DNA, 20 bp, synthetic construct
  misc_feature 1..20: note = Description of Artificial Sequence: Synthetic oligonucleotide
  source 1..20: mol_type = other DNA
agaccagactgagcaagaga

SEQ ID NO: 227 - DNA, 20 bp, synthetic construct
  misc_feature 1..20: note = Description of Artificial Sequence: Synthetic oligonucleotide
  source 1..20: mol_type = other DNA
gagccagaatgagcacgtga

SEQ ID NO: 228 - DNA, 20 bp, synthetic construct
  misc_feature 1..20: note = Description of Artificial Sequence: Synthetic oligonucleotide
  source 1..20: mol_type = other DNA
gaacacaatgcatagattgc

SEQ ID NO: 229 - DNA, 20 bp, synthetic construct
  misc_feature 1..20: note = Description of Artificial Sequence: Synthetic oligonucleotide
  source 1..20: mol_type = other DNA
gcagtctatgctttatgttt

SEQ ID NO: 230 - DNA, 20 bp, synthetic construct
  misc_feature 1..20: note = Description of Artificial Sequence: Synthetic oligonucleotide
  source 1..20: mol_type = other DNA
tgcactgcggccggaggagg

SEQ ID NO: 231 - DNA, 20 bp, synthetic construct
  misc_feature 1..20: note = Description of Artificial Sequence: Synthetic oligonucleotide
  source 1..20: mol_type = other DNA
ggctctgcggctggaggggg

SEQ ID NO: 232 - DNA, 20 bp, synthetic construct
  misc_feature 1..20: note = Description of Artificial Sequence: Synthetic oligonucleotide
  source 1..20: mol_type = other DNA
ggcacgacggctggaggtgg

SEQ ID NO: 233 - DNA, 20 bp, synthetic construct
  misc_feature 1..20: note = Description of Artificial Sequence: Synthetic oligonucleotide
  source 1..20: mol_type = other DNA
ggcatcacggctggaggtgg

SEQ ID NO: 234 - DNA, 20 bp, synthetic construct
  misc_feature 1..20: note = Description of Artificial Sequence: Synthetic oligonucleotide
  source 1..20: mol_type = other DNA
ggcgctgcggcgggaggtgg

SEQ ID NO: 235 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 236 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 237 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 238 - DNA, 20 bp, synthetic construct
  misc_feature 1..20: note = Description of Artificial Sequence: Synthetic oligonucleotide
  source 1..20: mol_type = other DNA
gaagcgcctggcagtgtacc

SEQ ID NO: 239 - DNA, 20 bp, synthetic construct
  misc_feature 1..20: note = Description of Artificial Sequence: Synthetic oligonucleotide
  source 1..20: mol_type = other DNA
ggcccagactgagcacgtga

SEQ ID NO: 240 - DNA, 20 bp, synthetic construct
  misc_feature 1..20: note = Description of Artificial Sequence: Synthetic oligonucleotide
  source 1..20: mol_type = other DNA
gaacacaaagcatagactgc

SEQ ID NO: 241 - DNA, 20 bp, synthetic construct
  misc_feature 1..20: note = Description of Artificial Sequence: Synthetic oligonucleotide
  source 1..20: mol_type = other DNA
ggcactgcggctggaggtgg

SEQ ID NO: 242 - DNA, 15 bp, synthetic construct
  misc_feature 1..15: note = Description of Artificial Sequence: Synthetic oligonucleotide
  source 1..15: mol_type = other DNA
gtgatcatcatcacc

SEQ ID NO: 243 - DNA, 15 bp, synthetic construct
  misc_feature 1..15: note = Description of Artificial Sequence: Synthetic oligonucleotide
  source 1..15: mol_type = other DNA
tggactgagtggctc

SEQ ID NO: 244 - DNA, 20 bp, synthetic construct
  misc_feature 1..20: note = Description of Artificial Sequence: Synthetic oligonucleotide
  source 1..20: mol_type = other DNA
gtagaggaggtggttcagga

SEQ ID NO: 245 - DNA, 20 bp, synthetic construct
  misc_feature 1..20: note = Description of Artificial Sequence: Synthetic oligonucleotide
  source 1..20: mol_type = other DNA
ttagagcaggtggttcagga

SEQ ID NO: 246 - DNA, 20 bp, synthetic construct
  misc_feature 1..20: note = Description of Artificial Sequence: Synthetic oligonucleotide
  source 1..20: mol_type = other DNA
caatacaaaggatagactgc

SEQ ID NO: 247 - DNA, 20 bp, synthetic construct
  misc_feature 1..20: note = Description of Artificial Sequence: Synthetic oligonucleotide
  source 1..20: mol_type = other DNA
ggagagagagcatagactgc

SEQ ID NO: 248 - DNA, 20 bp, synthetic construct
  misc_feature 1..20: note = Description of Artificial Sequence: Synthetic oligonucleotide
  source 1..20: mol_type = other DNA
taagacacaatgatgagtca

SEQ ID NO: 249 - DNA, 20 bp, synthetic construct
  misc_feature 1..20: note = Description of Artificial Sequence: Synthetic oligonucleotide
  source 1..20: mol_type = other DNA
ctggagatattgatgagtca

SEQ ID NO: 250 - DNA, 20 bp, synthetic construct
  misc_feature 1..20: note = Description of Artificial Sequence: Synthetic oligonucleotide
  source 1..20: mol_type = other DNA
aggttggcccaggccagggc

SEQ ID NO: 251 - DNA, 20 bp, synthetic construct
  misc_feature 1..20: note = Description of Artificial Sequence: Synthetic oligonucleotide
  source 1..20: mol_type = other DNA
cagtggacccaggccagggc

SEQ ID NO: 252 - DNA, 20 bp, synthetic construct
  misc_feature 1..20: note = Description of Artificial Sequence: Synthetic oligonucleotide
  source 1..20: mol_type = other DNA
tttgatgaggaggttcagga

SEQ ID NO: 253 - DNA, 20 bp, synthetic construct
  misc_feature 1..20: note = Description of Artificial Sequence: Synthetic oligonucleotide
  source 1..20: mol_type = other DNA
ccaaacaaaacatagactgc

SEQ ID NO: 254 - DNA, 20 bp, synthetic construct
  misc_feature 1..20: note = Description of Artificial Sequence: Synthetic oligonucleotide
  source 1..20: mol_type = other DNA
ggtgagagaaagatgagtca

SEQ ID NO: 255 - DNA, 20 bp, synthetic construct
  misc_feature 1..20: note = Description of Artificial Sequence: Synthetic oligonucleotide
  source 1..20: mol_type = other DNA
ttgtggacccaggccagggc

SEQ ID NO: 256 - DNA, 20 bp, synthetic construct
  misc_feature 1..20: note = Description of Artificial Sequence: Synthetic oligonucleotide
  source 1..20: mol_type = other DNA
gatgagataatgatgagtca

SEQ ID NO: 257 - DNA, 20 bp, synthetic construct
  misc_feature 1..20: note = Description of Artificial Sequence: Synthetic oligonucleotide
  source 1..20: mol_type = other DNA
cacagaagataccgagactg

SEQ ID NO: 258 - DNA, 720 bp, synthetic construct
  misc_feature 1..720: note = Description of Artificial Sequence: Synthetic polynucleotide
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcggccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 259 - DNA, 1518 bp, synthetic construct
  misc_feature 1..1518: note = Description of Artificial Sequence: Synthetic polynucleotide
  source 1..1518: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagccccgggagggcagaggaagtcttctaacatgcggtgacgtggaggagaatcccggccctactagttgatggggtggttcaggaggggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 260 - DNA, 1518 bp, synthetic construct
  misc_feature 1..1518: note = Description of Artificial Sequence: Synthetic polynucleotide
  source 1..1518: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagccccgggagggcagaggaagtcttctaacatgcggtgacgtggaggagaatcccggccctactagttgatgaggtggttcaggaggggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 261 - DNA, 23 bp, synthetic construct
  misc_feature 1..23: note = Description of Artificial Sequence: Synthetic oligonucleotide
  source 1..23: mol_type = other DNA
gttgatgaggtggttcaggaggg

SEQ ID NO: 262 - DNA, 3490 bp, synthetic construct
  misc_feature 1..3490: note = Description of Artificial Sequence: Synthetic polynucleotide
  source 1..3490: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacccacggcgtgcagtgcttgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 263 - DNA, 23 bp, synthetic construct
  misc_feature 1..23: note = Description of Artificial Sequence: Synthetic oligonucleotide
  source 1..23: mol_type = other DNA
ggcccagactgagcacgtgatgg

SEQ ID NO: 264 - DNA, 23 bp, synthetic construct
  misc_feature 1..23: note = Description of Artificial Sequence: Synthetic oligonucleotide
  source 1..23: mol_type = other DNA
ggtccagactgagcacgtgatgg

SEQ ID NO: 265 - DNA, 23 bp, synthetic construct
  misc_feature 1..23: note = Description of Artificial Sequence: Synthetic oligonucleotide
  source 1..23: mol_type = other DNA
ggctcagactgagcacgtgatgg

SEQ ID NO: 266 - DNA, 23 bp, synthetic construct
  misc_feature 1..23: note = Description of Artificial Sequence: Synthetic oligonucleotide
  source 1..23: mol_type = other DNA
ggcctagactgagcacgtgatgg

SEQ ID NO: 267 - DNA, 23 bp, synthetic construct
  misc_feature 1..23: note = Description of Artificial Sequence: Synthetic oligonucleotide
  source 1..23: mol_type = other DNA
ggcccagattgagcacgtgatgg

SEQ ID NO: 268 - DNA, 23 bp, synthetic construct
  misc_feature 1..23: note = Description of Artificial Sequence: Synthetic oligonucleotide
  source 1..23: mol_type = other DNA
ggttcagactgagcacgtgatgg

SEQ ID NO: 269 - DNA, 23 bp, synthetic construct
  misc_feature 1..23: note = Description of Artificial Sequence: Synthetic oligonucleotide
  source 1..23: mol_type = other DNA
ggtctagactgagcacgtgatgg

SEQ ID NO: 270 - DNA, 23 bp, synthetic construct
  misc_feature 1..23: note = Description of Artificial Sequence: Synthetic oligonucleotide
  source 1..23: mol_type = other DNA
ggtccagattgagcacgtgatgg

SEQ ID NO: 271 - DNA, 23 bp, synthetic construct
  misc_feature 1..23: note = Description of Artificial Sequence: Synthetic oligonucleotide
  source 1..23: mol_type = other DNA
ggcttagactgagcacgtgatgg

SEQ ID NO: 272 - DNA, 23 bp, synthetic construct
  misc_feature 1..23: note = Description of Artificial Sequence: Synthetic oligonucleotide
  source 1..23: mol_type = other DNA
ggctcagattgagcacgtgatgg

SEQ ID NO: 273 - DNA, 23 bp, synthetic construct
  misc_feature 1..23: note = Description of Artificial Sequence: Synthetic oligonucleotide
  source 1..23: mol_type = other DNA
ggcctagattgagcacgtgatgg

SEQ ID NO: 274 - DNA, 23 bp, synthetic construct
  misc_feature 1..23: note = Description of Artificial Sequence: Synthetic oligonucleotide
  source 1..23: mol_type = other DNA
ggtttagactgagcacgtgatgg

SEQ ID NO: 275 - DNA, 23 bp, synthetic construct
  misc_feature 1..23: note = Description of Artificial Sequence: Synthetic oligonucleotide
  source 1..23: mol_type = other DNA
ggcttagattgagcacgtgatgg

SEQ ID NO: 276 - DNA, 23 bp, synthetic construct
  misc_feature 1..23: note = Description of Artificial Sequence: Synthetic oligonucleotide
  source 1..23: mol_type = other DNA
ggtctagattgagcacgtgatgg

SEQ ID NO: 277 - DNA, 23 bp, synthetic construct
  misc_feature 1..23: note = Description of Artificial Sequence: Synthetic oligonucleotide
  source 1..23: mol_type = other DNA
ggttcagattgagcacgtgatgg

SEQ ID NO: 278 - DNA, 23 bp, synthetic construct
  misc_feature 1..23: note = Description of Artificial Sequence: Synthetic oligonucleotide
  source 1..23: mol_type = other DNA
ggtttagattgagcacgtgatgg

SEQ ID NO: 279 - DNA, 23 bp, synthetic construct
  misc_feature 1..23: note = Description of Artificial Sequence: Synthetic oligonucleotide
  source 1..23: mol_type = other DNA
ggcccagactgagcacgtgatgg

SEQ ID NO: 280 - DNA, 23 bp, synthetic construct
  misc_feature 1..23: note = Description of Artificial Sequence: Synthetic oligonucleotide
  source 1..23: mol_type = other DNA
ggtccagactgagcacgtgatgg

SEQ ID NO: 281 - DNA, 23 bp, synthetic construct
  misc_feature 1..23: note = Description of Artificial Sequence: Synthetic oligonucleotide
  source 1..23: mol_type = other DNA
ggctcagactgagcacgtgatgg

SEQ ID NO: 282 - DNA, 23 bp, synthetic construct
  misc_feature 1..23: note = Description of Artificial Sequence: Synthetic oligonucleotide
  source 1..23: mol_type = other DNA
ggcctagactgagcacgtgatgg

SEQ ID NO: 283 - DNA, 23 bp, synthetic construct
  misc_feature 1..23: note = Description of Artificial Sequence: Synthetic oligonucleotide
  source 1..23: mol_type = other DNA
ggcccagattgagcacgtgatgg

SEQ ID NO: 284 - DNA, 23 bp, synthetic construct
  misc_feature 1..23: note = Description of Artificial Sequence: Synthetic oligonucleotide
  source 1..23: mol_type = other DNA
ggttcagactgagcacgtgatgg

SEQ ID NO: 285 - DNA, 23 bp, synthetic construct
  misc_feature 1..23: note = Description of Artificial Sequence: Synthetic oligonucleotide
  source 1..23: mol_type = other DNA
ggtctagactgagcacgtgatgg

SEQ ID NO: 286 - DNA, 23 bp, synthetic construct
  misc_feature 1..23: note = Description of Artificial Sequence: Synthetic oligonucleotide
  source 1..23: mol_type = other DNA
ggtccagattgagcacgtgatgg

SEQ ID NO: 287 - DNA, 23 bp, synthetic construct
  misc_feature 1..23: note = Description of Artificial Sequence: Synthetic oligonucleotide
  source 1..23: mol_type = other DNA
ggcttagactgagcacgtgatgg

SEQ ID NO: 288 - DNA, 23 bp, synthetic construct
  misc_feature 1..23: note = Description of Artificial Sequence: Synthetic oligonucleotide
  source 1..23: mol_type = other DNA
ggctcagattgagcacgtgatgg

SEQ ID NO: 289 - DNA, 23 bp, synthetic construct
  misc_feature 1..23: note = Description of Artificial Sequence: Synthetic oligonucleotide
  source 1..23: mol_type = other DNA
ggcctagattgagcacgtgatgg

SEQ ID NO: 290 - DNA, 23 bp, synthetic construct
  misc_feature 1..23: note = Description of Artificial Sequence: Synthetic oligonucleotide
  source 1..23: mol_type = other DNA
ggtttagactgagcacgtgatgg

SEQ ID NO: 291 - DNA, 23 bp, synthetic construct
  misc_feature 1..23: note = Description of Artificial Sequence: Synthetic oligonucleotide
  source 1..23: mol_type = other DNA
ggcttagattgagcacgtgatgg

SEQ ID NO: 292 - DNA, 23 bp, synthetic construct
  misc_feature 1..23: note = Description of Artificial Sequence: Synthetic oligonucleotide
  source 1..23: mol_type = other DNA
ggtctagattgagcacgtgatgg

SEQ ID NO: 293 - DNA, 23 bp, synthetic construct
  misc_feature 1..23: note = Description of Artificial Sequence: Synthetic oligonucleotide
  source 1..23: mol_type = other DNA
ggttcagattgagcacgtgatgg

SEQ ID NO: 294 - DNA, 23 bp, synthetic construct
  misc_feature 1..23: note = Description of Artificial Sequence: Synthetic oligonucleotide
  source 1..23: mol_type = other DNA
ggtttagattgagcacgtgatgg

SEQ ID NO: 295 - DNA, 23 bp, synthetic construct
  misc_feature 1..23: note = Description of Artificial Sequence: Synthetic oligonucleotide
  source 1..23: mol_type = other DNA
gaagcgcctggcagtgtaccagg

SEQ ID NO: 296 - DNA, 23 bp, synthetic construct
  misc_feature 1..23: note = Description of Artificial Sequence: Synthetic oligonucleotide
  source 1..23: mol_type = other DNA
gaagtgcctggcagtgtaccagg

SEQ ID NO: 297 - DNA, 23 bp, synthetic construct
  misc_feature 1..23: note = Description of Artificial Sequence: Synthetic oligonucleotide
  source 1..23: mol_type = other DNA
gaagcgtctggcagtgtaccagg

SEQ ID NO: 298 - DNA, 23 bp, synthetic construct
  misc_feature 1..23: note = Description of Artificial Sequence: Synthetic oligonucleotide
  source 1..23: mol_type = other DNA
gaagcgcttggcagtgtaccagg

SEQ ID NO: 299 - DNA, 23 bp, synthetic construct
  misc_feature 1..23: note = Description of Artificial Sequence: Synthetic oligonucleotide
  source 1..23: mol_type = other DNA
gaagtgtctggcagtgtaccagg

SEQ ID NO: 300 - DNA, 23 bp, synthetic construct
  misc_feature 1..23: note = Description of Artificial Sequence: Synthetic oligonucleotide
  source 1..23: mol_type = other DNA
gaagtgcttggcagtgtaccagg

SEQ ID NO: 301 - DNA, 23 bp, synthetic construct
  misc_feature 1..23: note = Description of Artificial Sequence: Synthetic oligonucleotide
  source 1..23: mol_type = other DNA
gaagcgtttggcagtgtaccagg

SEQ ID NO: 302 - DNA, 23 bp, synthetic construct
  misc_feature 1..23: note = Description of Artificial Sequence: Synthetic oligonucleotide
  source 1..23: mol_type = other DNA
gaagtgtttggcagtgtaccagg

SEQ ID NO: 303 - DNA, 23 bp, synthetic construct
  misc_feature 1..23: note = Description of Artificial Sequence: Synthetic oligonucleotide
  source 1..23: mol_type = other DNA
gaagcgcctggcagtgtaccagg

SEQ ID NO: 304 - DNA, 23 bp, synthetic construct
  misc_feature 1..23: note = Description of Artificial Sequence: Synthetic oligonucleotide
  source 1..23: mol_type = other DNA
gaagtgcctggcagtgtaccagg

SEQ ID NO: 305 - DNA, 23 bp, synthetic construct
  misc_feature 1..23: note = Description of Artificial Sequence: Synthetic oligonucleotide
  source 1..23: mol_type = other DNA
gaagcgtctggcagtgtaccagg

SEQ ID NO: 306 - DNA, 23 bp, synthetic construct
  misc_feature 1..23: note = Description of Artificial Sequence: Synthetic oligonucleotide
  source 1..23: mol_type = other DNA
gaagcgcttggcagtgtaccagg

SEQ ID NO: 307 - DNA, 23 bp, synthetic construct
  misc_feature 1..23: note = Description of Artificial Sequence: Synthetic oligonucleotide
  source 1..23: mol_type = other DNA
gaagtgtctggcagtgtaccagg

SEQ ID NO: 308 - DNA, 23 bp, synthetic construct
  misc_feature 1..23: note = Description of Artificial Sequence: Synthetic oligonucleotide
  source 1..23: mol_type = other DNA
gaagtgcttggcagtgtaccagg

SEQ ID NO: 309 - DNA, 23 bp, synthetic construct
  misc_feature 1..23: note = Description of Artificial Sequence: Synthetic oligonucleotide
  source 1..23: mol_type = other DNA
gaagcgtttggcagtgtaccagg

SEQ ID NO: 310 - DNA, 23 bp, synthetic construct
  misc_feature 1..23: note = Description of Artificial Sequence: Synthetic oligonucleotide
  source 1..23: mol_type = other DNA
gaagtgtttggcagtgtaccagg

SEQ ID NO: 311 - protein, 257 aa, synthetic construct
  REGION 1..257: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMKKTMGWEASSERMYPEDGALKGEIKQRLKLKDGGHYDAEVKTTYKAKKPVQLPGAYNVNIKLDITSHNEDYTIVEQYERAEGRHSTGGMDELYKPREGRGSLLTCGDVEENPGPTS

SEQ ID NO: 312 - protein, 257 aa, synthetic construct; identical to SEQ ID NO: 311
  REGION 1..257: note = Description of Artificial Sequence: Synthetic polypeptide

SEQ ID NO: 313 - protein, 240 aa, synthetic construct
  REGION 1..240: note = Description of Artificial Sequence: Synthetic polypeptide
MREQGRGAVHRGGAHPGRAGRRRKRPQVQRVRRGRGRCHLRQADPEVHLHHRQAARALAHPRDHPDLRRAVLQPLPRPHEAARLLQVRHARRLRPGAHHLLQGRRQLQDPRRGEVRGRHPGEPHRAEGHRLQGGRQHPGAQAGVQLQQPQRLYHGRQAEERHQGELQDPPQHRGRQRAARRPLPAEHPHRRRPRAAARQPLPEHPVRPEQRPQREARSHGPAGVRDRRRDHSRHGRAVQV

SEQ ID NO: 314 - protein, 236 aa, synthetic construct
  REGION 1..236: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 315 - DNA, 23 bp, synthetic construct
  misc_feature 1..23: note = Description of Artificial Sequence: Synthetic oligonucleotide
  source 1..23: mol_type = other DNA
gttgatggggtggttcaggaggg

SEQ ID NO: 316 - protein, 504 aa, synthetic construct
  REGION 1..504: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPREGRGSLLTCGDVEENPGPTSWGGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 317 - protein, 505 aa, synthetic construct
  REGION 1..505: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPREGRGSLLTCGDVEENPGPTSWWGGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 318 - protein, 503 aa, synthetic construct
  REGION 1..503: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPREGRGSLLTCGDVEENPGPTSGGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 319 - protein, 505 aa, synthetic construct; identical to SEQ ID NO: 317
  REGION 1..505: note = Description of Artificial Sequence: Synthetic polypeptide

SEQ ID NO: 320 - protein, 246 aa, synthetic construct
  REGION 1..246: note = Description of Artificial Sequence: Synthetic polypeptide
WGGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYYQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 321 - protein, 245 aa, synthetic construct
  REGION 1..245: note = Description of Artificial Sequence: Synthetic polypeptide
GGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYYQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 322 - protein, 6 aa, synthetic construct
  REGION 1..6: note = Description of Artificial Sequence: Synthetic peptide
WGGSGG

SEQ ID NO: 323 - protein, 5 aa, synthetic construct
  REGION 1..5: note = Description of Artificial Sequence: Synthetic peptide
GGSGG

SEQ ID NO: 324 - protein, 8 aa, synthetic construct
  REGION 1..8: note = Description of Artificial Sequence: Synthetic peptide
SWWGGSGG

SEQ ID NO: 325 - protein, 6 aa, synthetic construct
  REGION 1..6: note = Description of Artificial Sequence: Synthetic peptide
KRLAVY

SEQ ID NO: 326 - protein, 246 aa, synthetic construct
  REGION 1..246: note = Description of Artificial Sequence: Synthetic polypeptide
WGGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 327 - protein, 245 aa, synthetic construct
  REGION 1..245: note = Description of Artificial Sequence: Synthetic polypeptide
GGSGGACVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2026/0126430 A1
(43) Pub. Date: May 7, 2026

(54) TRANSIENT REPORTERS AND METHODS FOR BASE EDITING ENRICHMENT

(71) Applicant: ARIZONA BOARD OF REGENTS ON BEHALF OF ARIZONA STATE UNIVERSITY, Scottsdale, AZ (US)

(72) Inventors: David Brafman, Phoenix, AZ (US); Xiao Wang, Chandler, AZ (US); Nicholas Brookhouser, Tempe, AZ (US); Stefan Tekel, Tempe, AZ (US); Kylie Standage-Beier, Phoenix, AZ (US)

(21) Appl. No.: 19/278,542

(22) Filed: Jul. 23, 2025

Related U.S. Application Data
(62) Division of application No. 17/347,360, filed on Jun. 14, 2021, now Pat. No. 12,405,265.
(60) Provisional application No. 63/038,220, filed on Jun. 12, 2020.

(51) Int. Cl. G01N 33/50; C07K 14/435; C12N 9/22; C12N 15/11; C12N 15/90

(52) U.S. Cl. CPC G01N 33/5073; C07K 14/43595; C12N 9/22; C12N 15/11; C12N 15/907; C12N 2310/20; C12N 2800/80

(57) ABSTRACT

Provided herein are compositions and methods for real-time identification and isolation of base-edited cell populations. Also provided herein are methods for producing enriched isogenic lines of genetically modified cells, including base-edited human pluripotent stem cells. In particular, provided herein are methods utilizing transient expression of reporter proteins, the detectable signal of which is altered following base editing. Using the transient reporter with a base editor permits enrichment of isogenic populations of base-edited cells.

### CROSS-REFERENCE TO RELATED PATENT APPLICATIONS

The present application is a divisional application of U.S. application Ser. No. 17/347,360, filed Jun. 14, 2021, which claims the benefit of priority to U.S. Provisional Application No. 63/038,220, filed on Jun. 12, 2020, the content of which is incorporated herein by reference in its entirety.

### STATEMENT REGARDING FEDERALLY SPONSORED RESEARCH

This invention was made with government support under R01 GM106081, R01 GM121698, R01 GM131405 and R21 AG056706 awarded by the National Institutes of Health. The government has certain rights in the invention.

### SEQUENCE LISTING

The instant application contains a Sequence Listing which has been submitted electronically in ST26 format and is hereby incorporated by reference in its entirety. Said ST26 copy, created on Jan. 19, 2026, is named “112624_01526.xml” and is 477,294 bytes in size.

### BACKGROUND

The rapid advancement of CRISPR Cas-based technologies has allowed for the modification (i.e., deletion, mutation and insertion) of human cells at precise genomic locations. For applications in which precise editing of a single nucleotide is desired, the CRISPR Cas machinery can be used to introduce site-specific double-stranded breaks (DSB) followed by homology-directed repair (HDR) using an exogenous DNA template. However, HDR is inefficient in mammalian cells, especially in recalcitrant cells such as human pluripotent stem cells (hPSCs), and repair of DSB is predominantly achieved through non-homologous end joining (NHEJ). In addition, NHEJ results in insertion or deletion of nucleotides (indels), resulting in undesired disruption (e.g. frameshift mutations, premature stop codons, deletion) of the targeted genes.

As an alternative to standard gene editing approaches that require a DSB, several groups have reported the development of deaminase base editors that do not rely on HDR to introduce single nucleotide genomic changes. Broadly speaking, these base editors consist of a fusion of three components—a D10A nickase Cas endonuclease, cytidine deaminase (APOBEC1), and a DNA uracil glycosylase inhibitor (UGI). This complex is capable of converting cytosine to thymine (or adenine to guanine on the complementary strand) without the need for a DSB and homology repair template. Overall, genome modification through the use of base editors has been shown to result in formation of fewer indels when compared to HDR-based methods.

Despite the advantages that deaminase base editors offer, identification and isolation of cell populations that have been successfully edited remains challenging. Specifically, there is no readily detectable phenotype to distinguish edited from unedited cells. In turn, isolation of edited cell populations requires single cell isolation followed by downstream sequencing verification. Some progress has been made to help enrich for edited cells, such as co-transfecting plasmids with a fluorescent reporter and using flow cytometry to isolate reporter-positive cells. Similarly, base editors fused to fluorescent proteins have been used to enrich for edited cell populations. However, these techniques are only reporters of transfection (RoT) and do not report on base editing activity within a cell population. Accordingly, there remains a need in the art for materials and efficient methods for selecting and enriching for base-edited human cells. There also remains a need in the art for efficient methods for producing isogenic populations of base edited human cells, particularly human pluripotent stem cell populations having targeted genetic modifications.

### BRIEF SUMMARY OF THE DISCLOSURE

In a first aspect, a polynucleotide encoding one or more reporter polypeptides, the polynucleotide including a PAM site adjacent to a base that when the base is edited a change in a function or characteristic of the one or more reporter polypeptides occurs. The polynucleotide may encode at least one reporter polypeptide with at least 90% sequence identity to SEQ ID NO: 2, wherein the polynucleotide encodes histidine at amino acid at position number 66 relative to SEQ ID NO: 1, and encodes glycine at amino acid position number 72 relative to SEQ ID NO: 1. Alternatively, the polynucleotide may encode a reporter polypeptide with at least 90% sequence identity to one of SEQ ID NO: 316 or 318. In one alternative, the polynucleotide comprises a polynucleotide selected from the group consisting of SEQ ID NO: 258, 259 and 260.

In a second aspect, provided herein is a kit. The kit can comprise or consist essentially of a first nucleic acid sequence encoding one or more reporter proteins, wherein the first nucleic acid includes a PAM site adjacent to a base that when edited causes a change in a function or characteristic of the one or more reporter proteins; a second nucleic acid sequence encoding a first sgRNA adjacent to a protospacer adjacent motif (PAM), wherein the first sgRNA comprises a protospacer sequence and is complementary to a portion of the nucleic acid sequence encoding one or more reporter proteins; a third nucleic acid sequence encoding a second sgRNA adjacent to a protospacer adjacent motif (PAM), wherein the sgRNA comprises a protospacer sequence and is complementary to a portion of a gene of interest to be base edited or comprises a cloning site to allow insertion of a complementary portion of a gene of interest to be base edited; and a fourth nucleic acid sequence encoding a base editor. The base editor can be selected from a cytidine deaminase base editor, an adenine base editor, Cas9-mediated adenosine base editor, and a prime editor. One or more of the first, second, third, or fourth nucleic acids can be provided in one or more vectors. The vector can be an episomal vector. The reporter protein can be a fluorescent protein or a variant thereof, luciferase or a variant thereof, β-galactosidase (lacZ), chloramphenyl acetyltransferase (CAT), β-glucuronidase (GUS), secretory alkaline phosphatase (SEAP), a survival selection protein, or a reporter protein that directly or indirectly produces or catalyzes a colorimetric reaction. The fluorescent protein can be a green fluorescent protein (GFP), a blue fluorescent protein (BFP), red fluorescent protein (RFP), luciferase, or mCherry, or a variant thereof. The fluorescent protein can be a BFP variant comprising a histidine at amino acid position 66 (numbered relative to SEQ ID NO:1). Alternatively, the reporter protein may be a fusion protein of two fluorescent proteins linked via a linker including at least one stop codon and a PAM site. The fourth nucleic acid sequence encoding a base editor can be a vector comprising a base editor operably linked to a heterologous promoter.

In another aspect, provided herein is a method for selecting a base edited cell. The method can comprise or consist essentially of introducing into a cell a first nucleic acid sequence encoding one or more reporter proteins, a second nucleic acid sequence encoding a first sgRNA adjacent to a protospacer adjacent motif (PAM), wherein the first sgRNA comprises a protospacer sequence and is complementary to a portion of the nucleic acid sequence encoding one or more reporter proteins; a third nucleic acid encoding a second sgRNA adjacent to a protospacer adjacent motif (PAM), wherein the second sgRNA comprises a protospacer adjacent sequence and is complementary to a portion of a gene of interest to be base edited; and a fourth nucleic acid sequence encoding a base editor, wherein the first nucleic acid includes a PAM site adjacent to a base that when edited causes a change in a function or characteristic of the one or more reporter proteins and wherein the change in function or characteristic results in a detectable signal; culturing the cell for about 48 hours to about 72 hours under conditions sufficient for expression of proteins encoded by the first, second, third and fourth nucleic acid sequences; sorting cells based on the presence or absence of a detectable signal, wherein a change in the detectable signal indicates that the base editor caused a base-to-base conversion or other genetic modification in the first nucleic acid sequence; and selecting cells exhibiting the changed detectable signal from the sorted cells, thereby selecting base edited cells. The base editor can be selected from a cytidine deaminase base editor, an adenine base editor, Cas9-mediated adenosine base editor, and a prime editor. One or more of the first, second, and third nucleic acids can be provided in a vector. The vector can be an episomal vector. The reporter protein can be a fluorescent protein or a variant thereof, luciferase or a variant thereof, β-galactosidase (lacZ), chloramphenyl acetyltransferase (CAT), β-glucuronidase (GUS), secretory alkaline phosphatase (SEAP), a survival selection protein, or a reporter protein that directly or indirectly produces or catalyzes a colorimetric reaction. The fluorescent protein can be a green fluorescent protein (GFP), a blue fluorescent protein (BFP), red fluorescent protein (RFP), luciferase, mCherry, or a variant or combination thereof. The fluorescent protein can be a BFP variant comprising a histidine at amino acid position 66 (numbered relative to SEQ ID NO: 1). The cell can be a human cell. The human cell can be a human pluripotent stem cell. The human pluripotent stem cell can be a human induced pluripotent stem cell obtained from a somatic cell of a human subject having a disease-associated single nucleotide polymorphism. Selecting can be performed using flow cytometry. Sorting can be performed using a fluorescence activated cell sorter (FACS).

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A-IF demonstrate conversion of BFP to GFP enables detection of base-editing activity in cells. (A) A mutant BFP was designed to convert to GFP upon a C-to-T nucleotide conversion. The protospacer sequence (underlined black) for the sgRNA, sg (BG), targeting the ‘CAC’ codon (underlined blue) resulting in a C-to-T conversion to ‘TAC’ (underlined green) and the corresponding amino acid change of histidine (blue) to tyrosine (green) at the 66th amino acid position in BFP. A PAM (underlined red) was placed in the position to orient the base editing window (underlined orange) around the C nucleotide (red) to facilitate BFPH66 to GFPY66 conversion. All alternative C-to-T conversions in the editing window resulted in silent mutations of the coding sequence. (B) The BFP mutant was cloned into a vector, pEF-BFP, with a human EF1α promoter driving expression. Targeting pEF-BFP with a cytidine deaminase base editor results in a C-to-T conversion causing a shift in the fluorescent emission spectra from BFP to GFP. (C) Representative fluorescent microscopy images of HEK293 cells transfected with pEF-BFP, pCMV-BE4-Gam and sg(NT) (top row) or sg(BG) (bottom row). (D) Representative flow cytometry plots of HEK293 cells transfected with pEF-BFP, pCMV-BE4-Gam and sg(NT) (top) or sg(BG) (bottom). Y-axis is a non-fluorescent control channel. (E) Schematic for isolation and detection of editing of episomal DNA after transfection. (F) Representative Sanger sequencing chromatogram of amplicons of episomal DNA isolated from HEK293 cells transfected with pEF-BFP, pCMV-BE4-Gam and sg(BG). The presence of T-nucleotide (red trace) at the target nucleotide (red asterisk) demonstrates the C-to-T base conversion responsible for the amino acid change of histidine to tyrosine at the 66th amino acid position and subsequent shift of the BFP emission spectra of the resultant protein to a GFP variant.

FIGS. 2A-2F demonstrate BFP-to-GFP conversion reports on base-editing at a chromosomal locus. (A) A pEF-BFP-PuroR vector was integrated into the C1ORF228 locus using homology-independent targeted integration to generate the HEK293-BFP cell line. (B) Schematic for plasmid or RNP base editing optimization using the HEK293-BFP line. (C) Representative fluorescent microscopy images of HEK293-BFP cells transfected with 600 ng pCMV-BE4-Gam and 200 ng sg(NT) (top row) or sg(BG) (bottom row). Scale bar=200 μm. (D) Editing efficiencies (GFP-positive cells) of HEK293-BFP cells transfected with various amounts of pCMV-BE4-Gam and ratios with the sg(BG) vector. n=3, *=P<0.05. (E) Representative fluorescent microscopy images of HEK293-BFP cells transfected with BE3-sg(BG) or -sg(NT) RNP complexes. (F) Schematic for RNP complex generation and transfection. BE3 was overexpressed, purified, complexed and validated in vitro, and transfected. Editing efficiencies (GFP-positive cells) of HEK293-BFP cells transfected with RNP complexes using various delivery reagents. n=3, *=P<0.05, **=P<0.01.

FIGS. 3A-3E demonstrate enrichment of base-edited cell populations using TREE. (A) Plasmid map of pDT-sgRNA vector that contains sg(BG) and sg(TS). Expression for both sgRNA cassettes is driven by separate U6 promoters (orange arrows). The BbsI restriction sites allow for direct restriction enzyme-based cloning of new target sites. (B) Schematic for enrichment of edited cells using TREE. HEK293 cells are co-transfected with pEF-BFP, pCMV-BE4-Gam and pDT-sgRNA vectors. After 48 h post-transfection, flow cytometry is used to sort cell populations into GFP-positive and -negative fractions. (C) Schematic for enrichment of edited cells using reporter of transfection (ROT). HEK293 cells are co-transfected with pEF-GFP, pCMV-BE4-Gam and sg(TS) vectors. After 48 h post-transfection, flow cytometry is used to sort cell populations into GFP-positive and -negative fractions. (D) Representative flow cytometry plots of (i) untransfected HEK293 cells and (ii) HEK293 cells transfected with pEF-BFP only as well as HEK293 cells in which TREE was applied targeting (iii) Site-1, (iv) Site-2 and (v) Site-3. (E) Quantification of base editing efficiency at Site-1, Site-2 and Site-3 in GFP-positive, GFP-negative and unsorted cell populations isolated using TREE- or ROT-based enrichment strategies. n=3; *=P<0.05, **=P<0.01.

FIGS. 4A-4C demonstrate TREE enables efficient multiplex base editing. (A) Plasmid map of pMT-sgRNA vector that contains sg(BG) in addition to sgRNA for multiple target sites. Expression for all sgRNA cassettes is driven by separate U6 promoters (orange arrows). The HindIII restriction site allows for additional sgRNAs for target sites to be cloned in through restriction enzyme-based cloning. (B) Quantification of multiplex base editing efficiency at Site-1, Site-2 and Site-3 in GFP-positive, GFP-negative and unsorted cell populations using TREE- or RoT-based enrichment strategies. n=3; *=P<0.05, **=P<0.01. (C) Clonal analysis of editing at multiple genomic loci using TREE. 40 GFP-positive clones were isolated via single-cell sorting. Editing was detected via PCR and Sanger sequencing. Blank icon indicates no editing observed, half-red icon indicates heterozygous C and T at the target site, and solid red icon indicates homozygous T edits at the genomic site.

FIGS. 5A-5D demonstrate highly efficient editing in human pluripotent stem cells (hPSCs) using TREE (A) Quantification of base editing efficiency (percentage GFP-positive cells) when hPSCs were co-transfected with pEF-BFP, sg(BG) and various base editing vectors. n=3; *=P<0.05, **=P<0.01. (B) Schematic for enrichment of edited hPSC using TREE. HPSCs were co-transfected with pEF-BFP, pEF-BE4-Gam/pEF-AncBE4 and pDT-sgRNA vectors. 48 h post-transfection, flow cytometry was used to sort cell populations into GFP-positive and -negative fractions. (C) Representative flow cytometry plots of (i) untransfected hPSCs cells and (ii) hPSCs transfected with pEF-BFP only as well as hPSCs cells in which TREE was applied targeting Site-1 utilizing (iii) pEF-BE4-Gam or (iv) pEF-AncBE4. (D) Quantification of base editing efficiency at Site-1 in GFP-positive, GFP-negative and unsorted cell populations isolated using TREE- or ROT-based enrichment strategies in which pEF-BE4-Gam or pEF-AncBE4 was employed. n=3; *=P<0.05, **=P<0.01.

FIGS. 6A-61 demonstrate efficient generation of isogenic hPSC lines using BIG-TREE. (A) Schematic for generation of clonal isogenic hPSC lines using BIG-TREE. HPSCs are co-transfected with pEF-BFP, pEF-AncBE4max, and pDT-sgRNA plasmid vectors. Forty-eight hours post transfection, FACS is used to isolate single GFP-positive cells into 96-well plates. Cells are subsequently expanded, and target clones are identified by Sanger sequencing of the target loci. (B) Schematic of vectors used for BIG-TREE- and RoT-based generation of clonal hPSC lines in which the APOE(158R) locus has been targeted. (C) Schematic of the APOE(158R) target locus in exon 4 of the APOE gene. Successful base editing of the APOE(158R) locus would result in a C-to-T conversion causing a change in the amino acid position at 158 from an arginine (APOE3) to a cysteine (APOE2). Representative Sanger sequences of the APOE(158R) locus of unedited parental hPSC lines as well as clonal hPSC lines that have been edited at the APOE(158R) are shown. Each line shown is representative of clones obtained from three independent parental hPSC populations (hPSC lines 1-3) with different genetic backgrounds. (D) Distribution of genotypes in clonal hPSCs derived from hPSC line 1 that was targeted at the APOE(158R) locus using BIG-TREE- or ROT-based methods. (E) Distribution of genotypes in clonal hPSCs derived from hPSC lines 2 and 3 that were generated via BIG-TREE-based targeting at the APOE(158R) locus. (F) Karyotype analysis of representative clones edited at the APOE(158R) locus. (G) Phase contrast images of representative clones edited at the APOE(158R) locus. (H) Immunofluorescence staining of representative clones edited at the APOE(158R) locus for pluripotency markers NANOG, OCT4, and SOX2. (I) Alpha fetaprotein (AFP), smooth muscle actin (SMA), and beta-III tubulin (TUJ1) immunofluorescence staining of representative clones edited at the APOE(158R) locus that had been subject to tri-lineage differentiation.

FIGS. 7A-7D demonstrate BIG-TREE-Based Gene Knockout of APOE in hPSCs. (A) Schematic of the APOE(39Q) locus in exon 3 of the APOE gene. Successful base editing of the APOE(39Q) locus would result in a C-to-T conversion causing a change in the amino acid at position 39 from a glutamine to a premature stop codon. (B) Representative Sanger sequencing of the APOE(39Q) locus in unedited wild-type cells (Q39/Q39; left panel) as well as hPSC clones in which a heterozygous (Q39/X39; middle panel) or homozygous (X39/X39; right panel) stop codon has been introduced. (C) Distribution of genotypes in clonal hPSCs that were generated via BIG-TREE targeting the APOE(39Q) locus. (D) Measurement of ApoE secretion in the condition medium of wild-type (Q39/Q39) and homozygous edited (X39/X39) hPSCs (n=3 independent experiments, p<0.05, two-tailed Student's t test).

FIGS. 8A-8C demonstrate simultaneous base editing of multiple loci in hPSCs using BIG-TREE. (A) Schematic of plasmid vectors used for BIG-TREE-based generation of clonal hPSC lines in which multiple loci have been simultaneously targeted. The pMT-sgRNA vector contains sg(BG) in addition to sgRNA for multiple target sites (S1, genomic site 1; S2, genomic site 2; S3, genomic site 3). (B) Representative Sanger sequencing chromatographs of the site 1, site 2, and site 3 loci in clonal hPSCs that have been generated via BIG-TREE multiplexed base editing. (C) Distribution of genotypes in clonal hPSCs that were generated via BIG-TREE multiplexed base editing.

FIG. 9 presents pDT-sgRNA sequence.

FIG. 10 is a schematic illustration of vector pDT-sgRNA.

FIG. 11 is a schematic illustration of vector pEF-AncBE4max.

FIG. 12 is a schematic illustration of vector pEF-BE4Gam. Additional vector map illustrations are provided in FIGS. 39-44.

FIGS. 13A-13E illustrate a fluorescent reporter system for real-time measurement of adenosine base editing activity. (a) The XMAS-TREE reporter vector consists of a human EF1α promoter driving expression of an mCherry cassette followed by a stop codon (TGA) then a GFP cassette. Targeting pEF-XMAS with an adenine base editor and sg(XMAS) will result in an A-to-G conversion, enabling expression of the downstream GFP reporter. (b) Two versions of pEF-XMAS-TREE plasmid were designed, one with a single stop codon (XMAS-1× Stop) and another with two stop codons (XMAS-2× Stop), preceding the coding sequence for GFP. The protospacer sequence (underlined black) for the sgRNA, sg(XMAS), targeting the TGA codon (underlined red) resulting in an A-to-G conversion to TGG and the corresponding amino acid change to tryptophan. The PAM sequence (underlined red) was placed to position the base editing window (underlined orange) around the target nucleotides. FIG. 13B discloses SEQ ID NOS 322, 200, 323, 201, 324, 202, and 324, respectively, in order of appearance. (c) Representative fluorescence microscopy images of HEK293 cells transfected with pEF-XMAS-1× Stop (left panels) or pEF-XMAS-2× Stop (right panels), pCMV-ABEmax, and sg(NT) (top panels) or sg(XMAS) (bottom panels). (d) Flow cytometry and (e) fluorescence microscopy analysis of HEK293 cells at various time points after transfection with pEF-XMAS-1× Stop (left panels) or pEF-XMAS-2× Stop (right panels), pCMVABEmax, and sg(XMAS).

FIGS. 14A-14B demonstrate identification and enrichment of base-edited cell populations using XMAS-TREE. (a) Schematic for identification and enrichment of adenosine base edited cells using XMAS-TREE. Cells are transfected with pEF-XMAS, pCMV-ABEmax, and pST-sgRNA vectors. Posttransfection, flow cytometry is used to sort cell populations into reporter-positive and -negative populations based upon mCherry and GFP expression levels. (b) Quantification of base editing efficiency at various genomic loci in unsorted (white bar), mCherry-negative/GFP-negative (grey bar), mCherry-positive/GFP-negative (red bar), and mCherry-positive/GFP-positive (orange bar) isolated cells using XMAS-TREE-based methods. Statistical comparisons were made between cells positive for the transfection reporter but not the editing reporter (i.e., mCherry-positive/GFP-negative) and cells positive for both (i.e. mCherry-positive/GFP-positive); *=p<0.05, **=p<0.01, ***=p<0.001.

FIGS. 15A-15C demonstrate that XMAS-TREE enables highly efficient multiplex adenosine base editing. (a) Cells were transfected with pEF-XMAS, pCMV-ABEmax, and a pMT-sgRNA that simultaneously targeted Site-1/Site-3/Site-4 or Site-5/HBG1/HBG2. Base editing was quantified in unsorted as well as reporter-positive and -negative cell populations. Statistical comparisons were made between cells positive for the transfection reporter but not the editing reporter (i.e. mCherry-positive/GFP-negative) and cells positive for both (i.e., mCherry-positive/GFP-positive); *=p<0.05, **=p<0.01, ***=p<0.001. (b) Schematic for employing XMAS-TREE for generation of clonal lines that have been simultaneously edited at multiple loci. HEK293 cells are co-transfected with pEF-XMAS, pCMV-ABEmax, and pMT-sgRNA. At 48 hours post-transfection, single mCherry-positive/GFP-positive cells are sorted into 96-well plates. After expansion, target clones are identified by Sanger sequencing at the target sites. (c) Analysis of clonal editing efficiency at multiple independent genomic sites using XMAS-TREE. A total of 30 clones were examined at each locus. White box indicates no editing observed a specified locus, half-filled box indicates mono-allelic targeting at the genomic site, and full box indicates bi-allelic editing at the target locus.

FIGS. 16A-16D demonstrate that XMAS-TREE can be employed for the highly efficient base editing of human pluripotent stem cells (hPSCs). (a) Representative fluorescence microscopy images of hPSCs transfected with pEF-XMAS-1× Stop (top panels) or pEF-XMAS-2× Stop (bottom panels), pEFABEmax, and sg(XMAS). (b) Flow cytometry and (c) fluorescence microscopy analysis of hPSCs at various time points after transfection with pEF-XMAS-1× Stop (top panels) or pEF-XMAS-2× Stop (bottom panels), pEF-ABEmax, and sg(XMAS). (d) Quantification of base editing efficiency at various genomic loci in unsorted (white bar), mCherry-negative/GFP-negative (grey bar), mCherry-positive/GFP-negative (red bar), and mCherry-positive/GFP-positive (orange bar) isolated hPSCs using XMAS-TREE-based methods. Statistical comparisons were made between hPSCs positive for the transfection reporter but not the editing reporter (i.e., mCherry-positive/GFP-negative) and cells positive for both (i.e. mCherry-positive/GFP-positive); *=p<0.05, **=p<0.01, ***=p<0.001.

FIGS. 17A-17C demonstrate highly efficient generation of clonal isogenic hPSC lines using XMAS-TREE. (a) Schematic for enrichment of adenosine base-edited cells using XMAS-TREE and reporter of transfection (ROT) based approaches. (b) Quantification of relative base editing in mCherry-positive cells isolated using ROT and mCherry-positive/GFP-positive cells isolated using XMASTREE. *=p<0.05, **=p<0.01, ***=p<0.001 (c) Analysis of clonal editing efficiency in hPSCs that were targeted at the Site-3 locus using XMAS-TREE- or ROT-based methods.

FIGS. 18A-18B demonstrate simultaneous adenosine base editing of multiple target sites in hPSCs using XMAS-TREE. (a) HPSCs were co-transfected with pEF-XMAS, pEF-ABEmax, and a pMT-sgRNA that simultaneously targeted Site-5/HBG1/HBG2. Flow cytometry was used to sort reporter-positive and -negative cell populations and base-editing was quantified at target loci. Statistical comparisons were made between cells positive for the transfection reporter but not the editing reporter (i.e., mCherry-positive/GFP-negative) and cells positive for both (i.e., mCherry-positive/GFP-positive); *=p<0.05, **=p<0.01, ***=p<0.001. (b) Quantification of relative base editing in mCherry-positive and mCherry-positive/GFP-positive cells isolated using ROT and XMAS-TREE, respectively.

FIG. 19 shows Sanger sequencing chromatographs of Site-1, Site-2, and Site-3 of GFP-positive, GFP-negative, and unsorted cell populations isolated with TREE- and RoT-based approaches.

FIG. 20 demonstrates that TREE allows for base editing of refractory APOE(R158) locus in HEK293 cells. (a) HEK293 cells were transfected with pEF-GFP, pCMV-BE4-Gam, and sg(TS). Comparison of trans-fection efficiency (percentage of GFP-positive cells) and editing efficiency (percentage of C-to-T conversion at target nucleotide) in unsorted cell populations at Site-1, Site-2, Site-2, and APOE(R158) locus. (b) Representative Sanger sequencing chromatographs of APOE(R158) locus in GFP-positive, GFP-negative, and unsorted cell populations isolated with RoT-based methods. FIG. 20B discloses SEQ ID NOS 325 and 210, respectively, in order of appearance. (c) Representative flow cytometry plot of HEK293 cells in which TREE was applied targeting the APOE(R158) locus. (d) HEK293 cells were transfected with pEF-BFP, pCMV-BE4-Gam, and pDT-sgRNA. Comparison of transfection efficiency (percentage of BFP-positive cells) and editing efficiency (percentage of C-to-T conversion at target nucleotide) in unsorted cell populations at Site-1, Site-2, Site-3, and APOE(R158) locus. (e) Representative Sanger sequencing chromatographs of APOE(R158) locus in GFP-positive, GFP-negative, and unsorted cell populations isolated with TREE-based methods. FIG. 20E discloses SEQ ID NOS 325 and 211, respectively, in order of appearance.

FIG. 21 demonstrates that TREE fluorescent output in HEK293 cells is transient. (a) HEK293 cells were transfected with pEF-BFP, pCMV-BE4-Gam, and pDT-sgRNA and GFP-positive cells were isolated by flow cytometry. Replated GFP-positive cells were analyzed by fluorescent microscopy and flow cytometry at various time points post-sorting. (b) Representative fluorescent microscopy images of cells prior to cell sorting (D-1, Pre-sort) and various time points (DO, D7, D10) after sorting. (c) Representative flow cytometry plots of (i) untransfected HEK293 cells, (ii) pEF-GFP transfected HEK293 cells, and (iii) TREE-enriched GFP-positive HEK293 cells 10 days after sorting.

FIG. 22 shows an Analysis of multiplexed edited HEK293 cells using TREE- and RoT-based methods. (a) Representative flow cytometry plot of HEK293 cells in which multiplex TREE was applied simultaneously targeting Site-1, Site-2, and Site-3. (b) Representative Sanger sequencing chromatographs of the Site-1, Site-2, and Site-3 loci in GFP-positive, GFP-negative, and unsorted cell populations isolated with TREE multiplex-based methods. (c) Comparison of base editing efficiencies at Site-1, Site-2, and Site-3 in GFP-positive, GFP-negative, and unsorted cell populations using TREE-based methods to target these sites individually or in a multiplexed manner. n=3; N.S.=not significant.

FIG. 23 shows an analysis of off-target sites in multiplexed edited HEK293 cells using TREE- and RoT-based methods. GFP-positive cell populations isolated from TREE and RoT approaches were PCR-amplified and subjected to Sanger sequencing on the top predicted off-target loci for the sgRNA sequences for sg(BG) and genomic Sites 1-3. The C nucleotides in red text are potential Cs that can undergo C-to-T conversion within the editing window in the protospacer. The numbers below each C are quantification of the percentage of Cs of the Sanger sequence chromatograms using EditR.

FIG. 24 depicts the identification of exclusive targeting events in clonal population in edited HEK293 cells using TREE. Representative Sanger sequencing chromatographs of clonal cell populations that contain edits exclusively at the target C and not any other Cs within the editing window.

FIG. 25 demonstrates that TREE allows for base editing in hPSCs. (a) Representative flow cytometry plots in which TREE was employed in hPSCs utilizing (i) untransfected (ii) pCMV-BE4-Gam or (iii) pCMV-AncBE4. (b) Editing efficiency (percentage GFP-positive cells) of targeting in hPSCs line with various amounts of pEF-AncBE4 plasmid and ratios with the sg(BG) vector. n=3, *=p<0.05. (c) Representative Sanger sequencing chromatographs of Site-1 in GFP-positive, GFP-negative, and unsorted cell populations isolated with TREE- and ROT-based methods in which pEF-BE4-Gam or pEF-AncBE4 was utilized. (d) Representative flow cytometry plot of hPSCs in which TREE was applied targeting the APOE(R158) locus. (e) Representative Sanger sequencing chromatographs of APOE(R158) locus in GFP-positive, GFP-negative, and unsorted cell populations isolated with TREE-based methods.

FIG. 26 shows that TREE fluorescent output in hPSCs is transient. Representative flow cytometry plots of (i) untransfected hPSCs, (ii) TREE-enriched GFP-positive hPSCs 0 days (iii) 14 days after sorting.

FIG. 27 demonstrates a Next generation sequencing (NGS) analysis of allelic outcomes at target sites in hPSCs. NGS analysis for the target site when TREE was applied to edit Site-1 or the APOE(R158) in hPSCs. The number to left of the allelic outcome indicates the position upstream (5′) relative to the PAM. Abbreviation: WT=wild-type unedited locus.

FIG. 28 depicts an Analysis of bystander editing in BIG-TREE edited hPSC clones. (a) Schematic of editing window and nucleotide position for target sites associated with FIG. 1 (APOE158R) and FIG. 2 (Genomic Sites 1-3). Red indicates target C within the editing window. Blue indicates bystander C within the editing window. (b) Distribution of bystander edits at the APOE(158R) locus in clonal hPSCs. (c) Distribution of bystander edits at genomic Sites1-3 in clonal hPSCs that were generated via multiplexed base editing.

FIG. 29 shows Off-target analysis of hPSC clones generated via BIG-TREE. Representative clonal lines were subjected to Sanger sequencing on the top predicted off-target loci for the sgRNA sequences for (a) sg(BG) and (b) sg(APOE158R).

FIG. 30 shows an Analysis of FAD-related genotypes in clonal hPSC lines generated from hPSC lines 2 and 3. Representative clonal lines derived from hPSC lines 2 and 3 were subject to Sanger sequencing to confirm that they retained the APP V7171 or PSEN1 A246E FAD-related mutations after editing at the APOE(158R) locus.

FIG. 31 shows Validation of BIG-TREE-based gene knock-out (KO) using a HEK293-mCherry line. (a) Schematic of using BIG-TREE to introduce stop codons into the genomically integrated mCherry cassette. Successful targeting with sg(mCh1), sg(mCh2), or sg(mCh3) will result in a C-to-T conversion causing a change in the amino acid at position 47, 114, or 168, respectively, from a glutamine (Q) to a premature stop codon (X). (b) Representative phase contrast (top panels) and fluorescent (bottom panels) images of HEK293-mCherry cells that had been targeted with a control non-targeting sgRNA [sg(NT)] or a sgRNA targeting mCherry [sg(mCh1), sg(mCh2), or sg(mCh3)]. (c) Representative flow cytometry plots of HEK293-mCherry cells that been targeted with a control non-targeting sgRNA [sg(NT)] or a sgRNA targeting mCherry [sg(mCh1), sg(mCh2), or sg(mCh3)]. (d) Sanger sequencing of sg(mCh1-3) protospacer sequences with codon translations indicated. Sequencing indicates conversion from target ‘CAG’ (Q) to ‘TAG’ (X). (e) Correlation between observed mCherry loss by flow cytometry and percent conversion of the target C to T within Sanger sequencing reads.

FIG. 32 shows that transfection efficiency is not predictive of editing efficiency. HEK293 cells were transfected with pEF-mCherry, pCMV-ABEmax, and sg(TS). Comparison of transfection efficiency (percentage of mCherry-positive cells) and editing efficiency (percentage of A-to-G conversion at target nucleotides) in unsorted cell populations targeted at various genomic loci.

FIG. 33 shows flow cytometry-based characterization of XMAS-TREE reporter. Representative flow cytometry plots of HEK293 cells transfected with pEF-XMAS-1× Stop or pEF-XMAS-2× Stop, pCMV-ABEmax, and sg(NT) or sg(XMAS).

FIG. 34 demonstrates a comparison of XMAS-TREE editing efficiency in individual- or multiplexed-targeted genomic sites. Quantification of base editing efficiencies at targeted loci mCherry-positive/GFP-positive cell populations using XMAS-TREE-based targeting in a single or multiplexed manner. N.S.=not significant, *=p<0.05.

FIG. 35 shows off-target analysis of HEK293 clones generated using XMAS-TREE-based methods. Representative clonal lines were analyzed by Sanger sequencing at the top predicted off-target loci for the sgRNA sequences for (a) sg(XMAS), (b) sg(Site-1), (c) sg(Site-3), and (d) sg(Site-4).

FIG. 36 shows the characterization of XMAS-TREE reporter in hPSCs. Representative flow cytometry of hPSCs transfected pEF-XMAS-1× Stop or pEF-XMAS-2× Stop, pEF-ABEmax, and sg(NT) or sg(XMAS).

FIG. 37 demonstrates Representative Sanger sequencing chromatographs of edited hPSCs enriched using XMAS-TREE and RoT-based approaches. Sanger sequencing chromatographs of Site-3 and PSEN1 of unsorted hPSCs as well as mCherry-positive and mCherry-positive/GFP-positive cells isolated using RoT-based and XMAS-TREE strategies, respectively.

FIG. 38 shows the distribution of genotypes in clonal hPSCs generated using XMAS-TREE-based methods. Analysis of clonal editing efficiency in hPSCs that were targeted at the PSEN1 locus.

FIG. 39 shows a vector map and sequence for pDT-sgRNA-1× Stop.

FIG. 40 shows a vector map and sequence for pDT-sgRNA-2× Stop.

FIG. 41 shows a vector map and sequence for pEF-XMAS-1× Stop.

FIG. 42 shows a vector map and sequence for pEF-XMAS-2× Stop.

FIG. 43 shows a vector map and sequence for pEF-ABE7.10.

FIG. 44 shows a vector map and sequence for pEF-ABEmax.

FIG. 45 shows sequences for mCherry-1× Stop-GFP and mCherry-2× Stop-GFP and XMAS 1× Stop reporter sequences, XMAS 2× Stop reporter sequences. FIG. 45 discloses SEQ ID NOS 311, 320, 312 and 321, respectively, in order of appearance.

FIG. 46 shows sequences for GFP and mCherry and the sequences of the XMAS 1× stop and XMAS 2× stop prior to and after editing. FIG. 46 discloses SEQ ID NOS 313-316, 326, 317-318, 327, and 319, respectively, in order of appearance.

### DETAILED DESCRIPTION

The compositions and methods provided herein are based at least in part on the Inventors' development of real-time, fluorescent-based methods for identification and isolation of base-edited cell populations. In particular, the disclosure provides a transient reporter for editing enrichment (TREE) to efficiently select and isolate base-edited cells from non-edited cells. As described herein, TREE takes advantage of a detectable change in a reporter protein signal as a direct reporter of base editing activity within a cell. Compared to conventional cell enrichment strategies that employ reporters of transfection (ROT), TREE significantly improved the editing efficiency at multiple independent loci, with efficiencies approaching 80%. Using these methods, it is possible to target multiple separate loci for gene editing and to identify and separate base edited cells from non-edited cells, all without homology directed repair (HDR). The combination of TREE with these base-editing methods yield isogenic genetically modified human pluripotent stem cell lines, with single-nucleotide editing efficiencies of >80% across multiple hPSC lines. Also described herein are methods that are particularly advantageous for efficient generation of loss-of-function and gain-of-function hPSC lines via introduction of premature stop codons or other genetic modifications, and for multiplex editing of hPSCs at several independent loci. These methods allow for the precise and efficient base editing of hPSCs for use in developmental biology, disease modeling, drug screening, and cell-based therapies.

Accordingly, in a first aspect, a polynucleotide encoding one or more reporter protein. The polynucleotide includes a PAM site adjacent to a base that when edited causes a change in a function or characteristic of the one or more reporter proteins. In one embodiment, the polynucleotide encodes a reporter protein with at least 90% sequence similarity to SEQ ID NO: 2, wherein the polynucleotide encodes histidine at amino acid at position number 66, relative to SEQ ID NO: 1 is provided, and encodes glycine at amino acid position number 72 relative to SEQ ID NO: 1. Alternatively, a reporter polypeptide may include two fluorescent proteins with at least 90% sequence identity to one of SEQ ID NO: 316 or 318. In another alternative, the polynucleotide comprises a polynucleotide selected from the group consisting of SEQ ID NO: 258, 259 and 260. The polypeptides may include a polypeptide having at least 80%, 85%, 90%, 95%, 96%, 97%, 98%, 99%, or 100% sequence identity to any one of the polypeptides provided herein. The polynucleotides encoding the polypeptides provided herein may include polynucleotides having at least 50%, 60%, 70%, 80%, 85%, 90%, 95%, 98%, 99% sequence identity to the polynucleotides provided herein. The polynucleotides encoding the polypeptides provided herein may be operably linked to a heterologous promoter. The polynucleotides may be included on constructs or in a vector, such as an expression vector, to allow for expression of the polypeptides in a cell.

The constructs and vectors provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed polynucleotides are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may effect transcription of the polynucleotides. Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons, such as lentiviral vectors. Vectors may carry genetic elements, such as those that confer resistance to certain drugs or chemicals.

Cells including any of the polynucleotides, constructs, or vectors described herein are provided. Suitable “cells” that may be used in accordance with the present invention include eukaryotic or prokaryotic cells. Suitable eukaryotic cells include, without limitation, plant cells, fungal cells, and animal cells. Suitable prokaryotic cells include, without limitation, gram-negative and gram-positive bacterial species.

In a second aspect provided herein, is a kit comprising nucleic acid sequences that encode components having base editing activity when introduced into a cell as well as components that, when expressed in the cell, function as a transient reporter of successful base editing. As described herein, the transient reporter-containing compositions and methods of their use enable enrichment of base edited cells.

In some cases, the kit comprises a one or more vectors comprising nucleic acid sequences that encode elements that perform base editing and the transient reporter of base editing enrichment. In some cases, the composition comprises one or more vectors comprising a first nucleic acid encoding one or more reporter proteins, wherein the first nucleic acid includes a PAM site adjacent to a base that when edited causes a change in a function or characteristic of the one or more reporter proteins, a second nucleic acid sequence encoding a base editing targeting cassette (preferably comprising one or more sgRNAs and protospacer adjacent motifs (PAMs) directed to the nucleic acid sequence that encodes the reporter protein), a third nucleic acid sequence encoding a base editing targeting cassette (preferably comprising one or more sgRNAs and protospacer adjacent motifs (PAMs) directed to a nucleic acid sequence of interest), and a fourth nucleic acid sequence encoding a base editor,

Any appropriate base editor capable of single nucleotide modifications without a need for double stranded DNA breaks can be used. In some cases, the base editor is a cytidine deaminase base editor (CBE) or adenine base editor (ABE). CBEs and ABEs install C·G-to-T·A and A·T-to-G·C transitions, respectively, and have been successfully used in many cell types including mammalian cells and plant cells. Cytidine deaminase base editors convert cytidine to uridine within a small editing window near the protospacer adjacent motif (PAM) site. Uridine is subsequently converted to thymidine through base excision repair, creating a C-to-T conversion (or G-to-A on the opposite strand). ABEs can convert adenine into inosine through deamination in any ABE protospacer (e.g. NGG, NG, and more) adjacent motif. The editing window will vary based on the type of base editor. For instance, the editing window for ABE is typically 12 to 17 nucleotides upstream of the ABE protospacer adjacent motif.

In some cases, the base editor is a prime editor. Prime editors are engineered Cas9 nickase-reverse transcriptase (RT) fusion proteins. When used in combination with prime editing guide RNAs (pegRNAs) that encode the desired edit, prime editors can edit bases in plant and animal cells without donor DNA or double-strand breaks. Unlike CBEs and ABE, prime editors are able to introduce point mutations, insertions, deletions, and all 12 possible base-to-base conversions. See Anzalone et al., i 576:149-157 (2019).

In some cases, the base editor is a Cas9-mediated adenosine base editor (referred to herein as “XMAS”). As described in Example 3, use of a Cas9-mediated adenosine base editor according to the methods of this disclosure introduces an A-to-G conversion. When a Cas9-mediated adenosine base editor used, for example, to target a TGA stop codon located between coding sequence for two different reporter proteins, the A-to-G conversion changes the TGA stop codon to TGG. Accordingly, expression of both reporter proteins allows for the real-time detection of adenosine base editing.

In some cases, the base editor is a dual adenine and cytosine base editor (e.g. CRISPR-Cas9-based synchronous programmable adenine and cytosine editor (SPACE), a codon-optimized fusion of cytosine deaminase PmCDA1, adenosine deaminase TadA, and a Cas9 nickase (Target-ACEmax), or a fusion of both adenine and cytosine deaminases with a Cas9 nickase (A&C-BEmax). When a dual editor is used, both C-T and A-G conversions happen simultaneously without the need for two base editor constructs.

In some cases, the base editor is any enzyme capable of modifying nucleobases is fused to a catalytically inactivated or impaired zinc finger nuclease (ZFN), or a transcription activator like effector (TALE). A ZF or TALE is designed to bind to a specific region of DNA, eliminating the potentially narrow editing window found with CRISPR systems.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

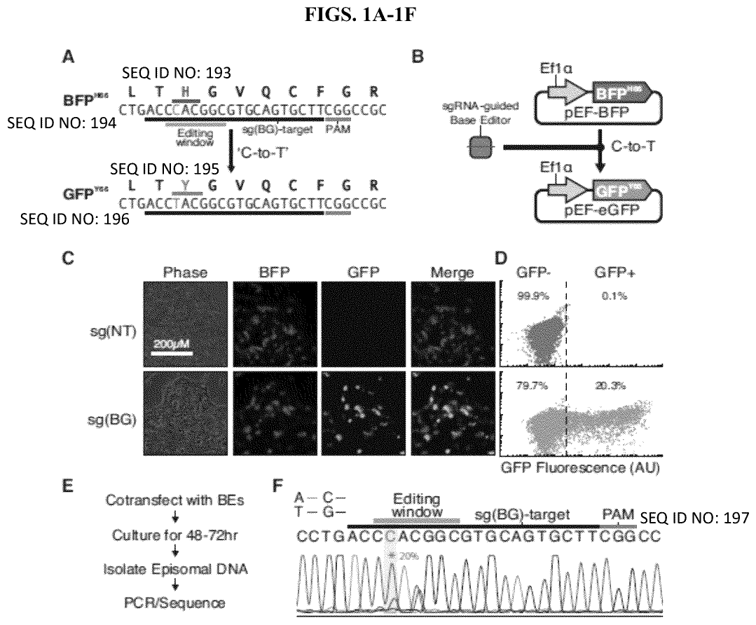

The reporter protein can be any detectable protein (e.g., detectable by fluorescence, color, bioluminescence indirect reporter system) for which a single base-to-base conversion or, in some cases, an insertion or deletion resulting in an observable change in the detectable protein. For example, the reporter protein can comprise a single nucleotide variation relative to the wild-type reporter protein. When a base-to-base conversion or other mutation is introduced into sequence encoding the reporter protein by base editing, the expressed reporter protein exhibits a detectable change (e.g., change in emitted fluorescent, change in color, change by way of indirect reporter) relative to the non-edited reporter protein. Examples of reporter proteins appropriate for this disclosure include, without limitation, fluorescent proteins (e.g., green fluorescent protein (GFP) and variants thereof, blue fluorescent protein (BFP) and variants thereof, red fluorescent protein (RFP) and variants thereof), luciferase and variants thereof, mCherry), β-galactosidase (lacZ), chloramphenyl acetyltransferase (CAT), β-glucuronidase (GUS), secretory alkaline phosphatase (SEAP), survival selection genes such as but not limited to antibiotic resistance, auxotrophies, flux redirection, toxin pumps, biosensors, reporter proteins that directly or indirectly produce or catalyze a colorimetric reaction, and those set forth in Table 1 below. In some cases, the reporter protein can be a blue fluorescent protein (BFP) variant that comprises a single nucleotide variation relative to the wild-type reporter protein. Referring to FIGS. 1A-IF, a blue fluorescent protein (BFP) variant can comprise a histidine at amino acid position 66 (numbered relative to SEQ ID NO:1) where the histidine is encoded by a C-A-C codon. In some cases, the reporter protein is a mutated, inactive form of luciferase. In such cases, it is advantageous to design a nucleic acid encoding the mutant luciferase such that a single base-to-base conversion or other genetic modification will introduce a corrective edit, resulting in expression of an active luciferase enzyme. In other embodiments the reporter protein is a fusion protein of two fluorescent proteins linked via a linker including at least one stop codon and a PAM site positioned to allow targeting and editing of the stop codon into a codon coding for an amino acid. Base editing allows for translation of the second reporter in the fusion protein and expression of the second reporter. The linker is positioned between the two reporter proteins such that expression of the reporter construct comprising the fusion protein in a cell results in expression of only the first reporter protein in the fusion protein. Once the polynucleotide encoding the fusion protein is edited, then both the first and second reporter will be detected. In this case the first reporter may be used as a measure of transfection efficiency and the second may be used to monitor and select for gene edited cells.

TABLE 1
Exemplary Reporter Proteins and Base Edits

| Gene Name: | Edit: | Function: | Notes: |
|---|---|---|---|
| Blue Fluorescent | C-to-T | Change | TREE |
| Protein (BFP) | (CBE) | emission | |
| | H66Y | spectra from | |
| | | 448 nm to 508 | |
| | | nm | |
| Green | A-to-G | Change | TREE |
| Fluorescent | (ABE) | emission | reaction run |
| Protein (GFP) | Y66H | spectra from | in reverse |
| | | 508 nm to 448 | |
| | | nm | |
| Activation of | A-to-G | Removal of | XMAS-TREE |
| fluorescent | (ABE) | stop codon in | |
| Proteins | Stop-to-W | coding | |
| | | sequence | |
| Introduction of | C-to-T | Addition of | BIG-TREE |
| premature stop | (CBE) | premature stop | |
| codons | Q-to-Stop | codons | |
| Activation of | A-to-G | Removal of | |
| antibiotic | (ABE) | stop codon in | |
| resistance | Stop-to-W | coding | |
| cassette | | sequence | |
| Activation of | A-to-G | Removal of | |
| auxotrophic | (ABE) | stop codon in | |
| selection marker | Stop-to-W | coding | |
| | | sequence | |
| Activation of | CBE or | Editing to | |
| gene function via | ABE | restore | |
| editing splice | | functional | |
| junctions | | splice junction | |
| | | in reporter | |

In some cases, the nucleic acid sequence that encodes a base editing cassette comprises a sgRNA adjacent to (e.g., located 5′ to) a protospacer adjacent motif (PAM), where the sgRNA comprises a protospacer sequence and targets a portion of the nucleic acid sequence encoding the reporter protein for base editing. As used herein, the term “base editing cassette” refers to an expression cassette or framework comprising nucleic acid sequence encoding a RNA oligonucleotide containing, in some cases, 18-22 base pairs (the single guide RNA or “sgRNA”) and PAM sequence. As used herein, a “single guide RNA” (sgRNA) is nucleotide sequence that is complementary to at least a portion of a target nucleic acid to be genetically modified by a base editor. Generally, a sgRNA comprises a nucleotide sequence that is partially or wholly complementary to a target sequence (such as a target genomic sequence or sequence in an expression vector) and comprises a target base pair. A gRNA target site also comprises a PAM located immediately downstream from the target site. In some cases, the PAM is a S. pyogenes Cas9 PAM ‘NGG.’ The PAM sequence may vary if other Cas9 variants are used. For some embodiments, the sgRNA preferably comprises a sequence of at least 10 contiguous nucleotides, and often a sequence of 18-22 contiguous nucleotides or more. In some embodiments, a sgRNA molecule can be from 20 to 300 or more bases in length, or more. In certain embodiments, a sgRNA molecule can be from 20 to 300 bases in length, or 20 to 120 bases, or 30 to 50 bases, or 39 to 46 bases. In some cases, no sgRNA is needed.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “target site” or “target sequence” refers to a genomic nucleic acid sequence that defines a portion of a nucleic acid to which biological molecules involved in base editing may specifically bind under conditions sufficient for binding to occur.

Preferably, nucleic acid sequences and vectors comprising sgRNAs are designed to allow for the facile cloning of new target sites via restriction enzyme digestion and ligation of oligonucleotides that target the desired genomic sequence. Depending on the PAM specificity and editing window configured in the base editing cassettes, the methods can be used in conjunction with any base editors or base editor variants. For instance, the PAM sequence and edit distance can be modified to match the editing specificity and window of the new base editor. Such modifications are straightforward to achieve with the BFP vector using TREE, or the stop codon between RFP and GFP using BIG-TREE.

By way of example, the sgRNA can be designed for editing of a nucleic acid sequence encoding a BFP variant as the reporter protein. As described in the Examples that follow, the sgRNA is designed to bring a cytidine deaminase base editor to the BFP variant-encoding nucleic acid sequence for base editing. Referring to FIG. 1A, the protospacer sequence (underlined black) for the sgRNA, sg(BG), targeting the ‘CAC’ codon (underlined blue) resulting in a C-to-T conversion to ‘TAC’ (underlined green) and the corresponding amino acid change of histidine (blue) to tyrosine (green) at the 66th amino acid position in BFP. A PAM (underlined red) was placed in the position to orient the base editing window (underlined orange) around the C nucleotide (red) to facilitate BFPH66 to GFPY66 conversion.

In some cases, the amino acid sequence for wild-type BFP is

(SEQ ID NO: 1)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDEKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK.

In some cases, the amino acid sequence for variant BFP is MVSKGEELFTGVVPILVELDGDVNGHKFSVSGEGEGDATYGKLTLKFICTTGKLPVPWP TLVTTLTHGVQCFGRYPDHMKQHDFFKSAMPEGYVQERTIFFKDDGNYKTRAEVKFEG DTLVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYK* (SEQ ID NO:2). In SEQ ID NO:2, the histidine at residue position 66 is underlined. The S-to-G modification to introduce the PAM site is double underlined.

Preferably, nucleic acid sequence encoding the reporter protein is operably linked to a promoter that drives expression of the reporter protein upon introduction into a cell. A promoter, generally, is a region of nucleic acid that initiates transcription of a nucleic acid encoding a product. A promoter may be located upstream (e.g., 0 bp to −100 bp, −30 bp, −75 bp, or −90 bp) from the transcriptional start site of a nucleic acid encoding a product, or a transcription start site may be located within a promoter. A promoter may have a length of 100-1000 nucleotide base pairs, or 50-2000 nucleotide base pairs. In some embodiments, promoters have a length of at least 2 kilobases (e.g., 2-5 kb, 2-4 kb, or 2-3 kb). The term “expression” as used herein is defined as the transcription and/or translation of a particular nucleotide sequence driven by its promoter. As used herein, the term “operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. As exemplified in FIG. 1B, the promoter can be human EF1α promoter, which is a strong constitutively active promoter, however other promoters can be used. In some cases the promoter is an inducible promoter or a cell-type specific promoter. Examples of base editing in different organisms and plus DOI citations for each example are set forth in Table 2.

TABLE 2
Examples of Base Editing in Different Organisms

| | C-T | A-G |
|---|---|---|
| bacteria | | |
| E. coli | 10.1038/ncomms13330, 10.1038/s42003-018-0035-5 | |
| B. melitensis | 10.1038/s42003-018-0035-5 | |
| Streptomyces | 10.1073/pnas.1913493116 | |
| C. glutamicum | 10.1002/bit.27121 | |
| yeast | | |
| S. cerevesiae | 10.1126/science.aaf8729 | |
| plants | | |
| rice, wheat, maize | 10.1038/nbt.3811 | |
| rice and tomato | 10.1038/nbt.3833 | |
| rice and wheat | 10.1038/s41587-020-0455-x | |
| mammalian | | |
| mouse embryo | 10.1038/s41598-018-33533-5, | |
| mice | 10.1038/s41556-018-0202-4, | |
| | 10.1038/nbt.3816 | |
| | 10.1038/nbt.4194 | |
| | 10.1038/nature26155, | |
| | 10.1038/541587-019-0134-y, | |
| | 10.1016/j.molcel.2017.08.008, | |
| | 10.1038/nbt.4102, | |
| | 10.1038/s41467-019-11562-6, | 10.1038/s41587-019-0050-1, |
| | 10.1038/ncomms15790, | 10.1038/nbt.4172, |
| | 10.1038/nbt.3803, | 10.1038/nature24644, |
| | 10.1038/nature17946, | 10.1016/j.ymthe.2019.05.013, |
| human cells | 10.1038/nmeth.4027 | 10.1038/s41587-020-0491-6 |
| mouse embryo | | 10.1038/s41467-018-04768-7 |
| Pig | 10.1038/s41467-019-10421-8 | |
| mice, mouse zygote, mammalian | | 10.1016/j.ymthe.2019.11.022 |
| mice and mice embryo | | 10.1038/nbt.4148 |
| mice organs | 10.1038/s41551-019-0501-5 | 10.1038/s41551-019-0501-5 |
| dual base editing | 10.1038/s41587-020-0527-y, | |
| | 10.1038/s41587-020-0509-0, | |
| | 10.1038/s41587-020-0535-y | |

As used herein, the term “vector” is intended to mean a nucleic acid molecule capable of transporting another nucleic acid. By way of example, appropriate vectors for the compositions and methods of this disclosure include episomal vectors, viral vectors (e.g., retrovirus, adenovirus, baculovirus), plasmids, RNA vectors, or linear or circular DNA or RNA molecules which may comprise or consist of a chromosomal, non-chromosomal, semi-synthetic, or synthetic nucleic acid. Large numbers of suitable vectors are known to those of skill in the art and commercially available. Preferred vectors are episomal vectors, which are capable of autonomous replication due to the presence of an origin of replication. Preferably, vectors of the base editing compositions described herein are episomal vectors, meaning they are capable of autonomous replication due to the presence of an origin of replication. While the nucleic acid sequences can be provided on separate vectors, it will be understood that it is possible to configure a single or pair of expression vectors comprising the base editing elements described herein.

### Methods

In another aspect, provided herein are methods for genome engineering (e.g., for altering or manipulating the expression of one or more genes or one or more gene products) in cells, in vitro, in vivo, or ex vivo. In particular, the methods provided herein are useful for targeted base editing or base correction in any cell.

In some cases, the methods comprise multiplex editing at multiple loci. In such cases, the methods described herein can be performed using a vector comprising dual-targeting sgRNAs. In such cases, a first nucleic acid sequence encodes a sgRNA for base editing in the reporter protein, and a second nucleic acid sequence encodes a sgRNA for a genomic target site. In some cases, methods described herein can be performed using a vector comprising a multiplex-targeting sgRNAs. In such cases, a first nucleic acid sequence encodes a sgRNA for base editing in the reporter protein, and a second, third, fourth, etc. nucleic acid sequence encodes a sgRNA for a genomic target site. When introduced into a cell with nucleic acid sequences encoding the base editor and reporter protein, it is possible to base edit two or more genomic target sites and also promote a base-to-base conversion or other genetic modification that yields a detectable change in fluorescent emission or color of the transient reporter protein. The Examples demonstrate successful base editing at two or more genomic target sites and plus introduction of a C-to-T conversion in nucleic acid sequence encoding a BFP variant, thus resulting in a shift in fluorescent emission spectra from BFP to GFP as described herein. In this manner, base-edited cells can be sorted and selected based on fluorescence emission spectra or other detectable signals. Accordingly, also provided herein are engineered cells that have been genetically modified according to these methods.

Although human pluripotent stem cells are exemplified herein, it will be understood by practitioners in the art that the base editing compositions and methods can be used with other cell types, including a variety of human cell types and cells of other types of animals. In some cases, the cell is a mammalian cell. Preferably, the mammalian cell is a human cell. Cells appropriate for use in the methods of this disclosure include, without limitation, pluripotent stem cells, multipotent cells, dissociated organs or organoids, terminally differentiated cells, immune cells, hematopoietic stem progenitor cells (HSPCs) (e.g., umbilical cord blood HSPCs), and fibroblasts. In addition to mammalian cells, it will be understood by practitioners in the art that the base editing compositions and methods can be used with other cell types such as bacteria cells, yeast cells, plant cells, and other single celled organisms.

As used herein, the term “pluripotent stem cell” (hPSC) means a cell capable of continued self-renewal and capable, under appropriate conditions, of differentiating into cells of all three germ layers. hPSCs exhibit a gene expression profile that includes SOX2′ and OCT4. Examples of human PSCs (hPSCs) include human embryonic stem cells (hESCs) and human induced pluripotent stem cells (hiPSCs). As used herein, “iPS cells” or “iPSCs” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells. In some cases, the modified cells are human pluripotent stem cells such as human embryonic stem cells or induced pluripotent stem cells. In some cases, the modified cells are human embryonic stem cells isolated from human embryonic tissues. In other cases, the modified cells are cells isolated from human blastocysts and then modified. In some cases, the modified cells are human placental or umbilical cord stem cells.

Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

Subject-specific somatic cells for reprogramming into induced pluripotent stem cells can be obtained or isolated from a target tissue of interest by biopsy or other tissue sampling methods. In some cases, subject-specific cells are manipulated in vitro prior to use in a method of this disclosure. For example, subject-specific cells can be expanded, differentiated, genetically modified, contacted to polypeptides, nucleic acids, or other factors, cryo-preserved, or otherwise modified prior to use according to the methods of this disclosure.

For the methods described herein, gene editing systems or components thereof (e.g., a vector encoding a base editor protein, a gRNA) are introduced into a cell (e.g., a human pluripotent stem cell). As used herein, the term “introducing” encompasses a variety of methods of introducing DNA into a cell, either in vitro or in vivo, such methods including transformation, transduction, transfection (e.g., electroporation), nucleofection (an electroporation-based transfection method which enables transfer of nucleic acids such as DNA and RNA into cells by applying a specific voltage and reagents) and infection. Where the introducing involves electroporation (e.g., nucleofection), a polynucleotide (e.g., a plasmid, a single stranded DNA, a minicircle DNA, RNA) is electroporated into a target cell. Vectors are useful for introducing DNA encoding molecules into cells. Any appropriate delivery vector can be used with the methods described herein. For example, delivery vectors include exosomes, viruses (viral vectors), and viral particles. Preferably, the delivery vector is a viral vector, such as a lenti- or baculo- or preferably adeno-viral/adeno-associated viral (AAV) vectors, but other non-viral means of delivery are known (such as yeast systems, microvesicles, gene guns/means of attaching vectors to gold nanoparticles). Other methods of introducing a nucleic acid into a host cell are known in the art, and any known method can be used to introduce a nucleic acid (e.g., vector or expression construct) into a cell for the methods provided herein. Suitable methods include, without limitation, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv. Drug Deliv. Rev.), and the like. Delivery of components may also include use of ribonucleoproteins complexed with sgRNA alone, or in combination with nucleic acid delivery.

A cell is “genome edited” or “genetically modified” if the cell includes a modification to its genome compared to a non-genome edited cell of the same type. In some cases, a non-genome edited cell is a wild-type cell. As used herein, the terms “genetically modified” and “genetically engineered” are used interchangeably and refer to a prokaryotic or eukaryotic cell that includes an exogenous polynucleotide, regardless of the method used for insertion. In some cases, a cell has been modified to comprise a non-naturally occurring nucleic acid molecule that has been created or modified by the hand of man (e.g., using recombinant DNA technology) or is derived from such a molecule (e.g., by transcription, translation, etc.). A cell that contains an exogenous, recombinant, synthetic, and/or otherwise modified polynucleotide is considered to be an engineered or “genome edited” cell. Genetically editing or modifying a cell refers to modifying cellular nucleic acid within a cell, including genetic modifications to endogenous and/or exogenous nucleic acids within the cell. Genetic modifications can comprise deletions, insertions, integrations of exogenous DNA, gene correction and/or gene mutation.

The term “substantially pure cell composition of genetically modified cells” as used herein refers to a cell composition comprising at least 70%, more preferentially at least 90%, most preferentially at least 95% of genetically modified cells in the cell composition obtained by methods of this disclosure. The terms “purified” or “enriched” cell populations are used interchangeably herein, and refer to cell populations, in vitro or ex vivo, that contain a higher proportion of a specified cell type or cells having a specified characteristic than are found in vivo (e.g., in a tissue).

As used herein, the term “isogenic” refers to cells or organisms that are genetically related, or having the same or closely similar genotypes, such as cells of a cell line. For example, cells of a clonal population of cells are isogenic to each other. In some cases, a first population of human pluripotent stem cells can have a wild-type, genetically unmodified genome, and a second population of pluripotent stem cells can be isogenic to the first population except that they have been genetically modified (which term as used herein includes progeny of modified cells) to comprise a particular genetic modification. Individual cells of the second population may be isogenic to each other if obtained by clonal expansion of a single genetically modified cell.

In some cases, cells into which nucleic acid sequences encoding a base editor, reporter protein, and base editing cassette have been introduced and then cultured for about 48 to about 72 hours are sorted using any sorting technique capable of detecting expression of green fluorescent protein and, optionally, other cell markers. Methods and techniques for assessing the expression and/or levels of cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In preferred embodiments, cells genetically modified according to the methods of this disclosure are detected and sorted by fluorescence-based flow cytometry. As used herein, the term “flow cytometry” refers to a cell analysis technique that detects and measures physical and chemical characteristics of a population of cells or particles in a rapidly flowing fluid stream as they pass in front of a viewing aperture. The term “flow cytometry” encompasses fluorescence-based flow cytometry in which, generally, lasers are used to detect and count cells based on fluorescence emission of fluorophores associated with the cells. In some cases, flow cytometry is performed using a specialized flow cytometer known as a fluorescence activated cell sorter (FACS). Cell sorters like FACS use fluidics and fluorescence components similar to those in flow cytometers, but are able to divert a specific population from within a heterogeneous sample into a separate tube, typically based on specified fluorescence characteristics.

Any appropriate technique can be used to as an additional means to confirm that base editing has occurred. For example, Sanger sequencing or next generation sequencing (NGS) can be used to detect C-to-T conversions.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

In another aspect, provided herein is a cell culture composition comprising the isogenic line of genetically modified human pluripotent stem cells produced according to the methods of this disclosure and a chemically defined culture medium. The term “chemically defined culture medium” or “chemically defined medium,” as used herein, means that the molecular identity, chemical structure, and quantity of each medium ingredient is definitively known. The term “ingredient,” as used herein, refers to a component the molecular identity and quantity of which is known. In some cases, a chemically defined medium is made xeno-free, and incorporates human proteins, which can be produced using recombinant technology or derived from placenta or other human tissues, in lieu of animal-derived proteins. In some embodiments, all proteins added to the medium are recombinant human proteins.

In some cases, the method is performed using human induced pluripotent stem cells obtained by reprogramming a somatic cell of a human subject, such as a human subject that has a genetic disorder caused by a single nucleotide polymorphism. In such cases, the resulting base-edited human cells are autologous to the human subject and, in some cases, can be administered back to the subject (e.g., cell therapy).

In another aspect, provided herein is an isogenic population of human cells genetically modified ex vivo to comprise a base-to-base conversion or other genetic modification. In some cases, the base-to-base conversion corrects or suppress a disease-associated single nucleotide polymorphism (SNP) or modifies an disease-associated isoform of a particular protein. As demonstrated in the Examples, the base editing-transient reporter enrichment methods of this disclosure were used to modify a nucleotide sequence encoding a APOE2 isoform of human APOE protein, such that the modified cell did not express any other isoform of the APOE protein. In this example, the human cells were induced pluripotent stem cells obtained from a somatic cell of a human subject having Familial Alzheimer's disease (FAD). Although correction of a SNP associated with FAD is exemplified herein, the methods of this disclosure can be used to correct introduce disease-correcting or disease-suppressing base edits in human cells provided that there is an appropriate PAM in the correct location downstream of the target SNP.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids and/or other constructs (including cell populations) described in this disclosure may be isolated. As used herein, “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions (e.g., cell population) described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

### Articles of Manufacture

In another aspect, provided herein is an article of manufacture such as a kit comprising a composition comprising expression vectors to perform base editing and editing enrichment using TREE as described herein. In certain embodiments, the kit comprises a plurality of vectors to achieve targeted base editing and for expression of the transient reporter (TREE). In some cases, the kit further comprises reagents and other materials useful for introducing vectors into cells and for culturing modified cells according to the methods. In some cases, the kit further comprises instructions for performing the methods of this disclosure.

In another aspect, provided herein is a kit for generating substantially pure populations of base-edited cells including, as a non-limiting example, base-edited human pluripotent stem cells. In exemplary embodiments, the kit comprises one or more of (i) a culture medium suitable for maintaining cells in vitro or ex vivo; (ii) base editing vectors as described herein; (iii) reagents for introduction of the base editing vectors into cells (e.g., transfection reagents, transduction reagents, electroporation reagents), and (iv) instructions describing a method for generating substantially pure populations of base-edited cells as described herein, the method employing one or more of the culture medium, the base editing vectors, and the reagents for introducing vectors into the cells. In some cases, the kit further comprises reagents and/or materials for flow cytometry and cell sorting using FACS.

All publications, including but not limited to patents and patent applications, cited in this specification are herein incorporated by reference as though set forth in their entirety in the present application.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. “About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated

All publications, including but not limited to patents and patent applications, cited in this specification are herein incorporated by reference as though set forth in their entirety in the present application.

### EXAMPLES

### Example 1—Transient Reporter for Editing Enrichment (TREE)

Current approaches to identify cell populations that have been modified with deaminase base editing technologies are inefficient and rely on downstream sequencing techniques. In this example, a blue fluorescent protein (BFP) that converts to green fluorescent protein (GFP) upon a C-to-T substitution was used as an assay to report directly on base editing activity within a cell. Using this assay, various base editing transfection parameters and delivery strategies were developed. Moreover, this assay was used in conjunction with flow cytometry to develop a transient reporter for editing enrichment (TREE) to efficiently purify base-edited cell populations. Compared to conventional cell enrichment strategies that employ reporters of transfection (RoT), TREE significantly improved the editing efficiency at multiple independent loci, with efficiencies approaching 80%. In addition, the BFP-to-GFP conversion assay was employed to develop base editor vector design for human pluripotent stem cells (hPSCs), a cell type that is resistant to genome editing and in which modification via base editors has not been previously reported. At last, using these optimized vectors in the context of TREE allowed for the highly efficient editing of hPSCs. It is envisioned that TREE is a readily adoptable method to facilitate base editing applications in synthetic biology, disease modeling, and regenerative medicine.

### Materials and Methods

Plasmid construction: Unless otherwise noted, all molecular cloning polymerase chain reactions (PCR) were performed using Phusion® High-Fidelity DNA polymerase (New England Biolabs, Ipswich, MA, USA) using the using the manufacturer's recommended protocols. All restriction enzyme (New England Biolabs) digests were performed according to the manufacturer's instructions. Ligation reactions were performed with T4 DNA Ligase (New England Biolabs) according to the manufacturer's instructions. PCR primers and oligonucleotides were synthesized by Integrated DNA Technologies (Coralville, IA, USA). All PCR products and intermediate plasmid products were confirmed via Sanger sequencing (DNASU Sequencing Core Facility and Genewiz).

For construction of the pEF-BFP plasmid, we utilized PCR to add the H-66 and protospacer adjacent motif (PAM) site mutations into a GFP cassette (Addgene #11154). PCR products containing these mutations were digested with SapI/EcoRI and SapI/NotI and ligated into a EcoRI/NotI digested EF1α expression vector (Addgene #11154).

For construction of the pDT-sgRNA vector, sgRNAs were synthesized as pairs of oligonucleotides (Table 4). Subsequently, 5′ phosphates were added to each oligonucleotide pair by incubating 1 μg oligonucleotide in 50 μl reactions containing 1×T4 DNA Ligase Buffer (New England Biolabs) and 10 units of T4 Polynucleotide Kinase at 37° C. overnight. Oligonucleotides were then duplexed by heating the kinase reactions to 90° C. on an aluminum heating block for 5 minutes followed by slowly returning the reaction to room temperature over 1 hour. Following duplexing, guides were cloned into a modified pSB1C3 vector containing a U6 promoter, inverted BbsI restriction enzyme digestion sites, and a Streptococcus pyogenes recognized sgRNA hairpin. For construction of pMT-sgRNA, pairs of sgRNAs (Table 4) were PCR amplified with primers adding EcoRI/SapI restriction enzyme digestion sites or SapI/XbaI restriction enzyme digestion sites. Purified PCR products were then digested with the respective restriction enzymes and ligated into EcoRI/XbaI digested pUC19 vector (Addgene #50005). The resultant vector contained pairs of sgRNA expression cassettes. To add additional sgRNA expression cassettes, pairs of sgRNAs were PCR amplified with primers that add HindIII/SapI or SapI/HindIII restriction enzyme digestion sites. These products were then digested with HindIII/SapI and ligated into HindIII digested and dephosphorylated pDT-sgRNA vector.

For insertion of the EF1α promoter into the pCMV-BE4-Gam (Addgene #100806) and pCMV-AncBE4max (Addgene #112094), EF1α was PCR amplified from an EF1α expression vector (Addgene #11154) adding SpeI/NotI restriction enzyme digestion sites. After purification and digestion, these PCR products were ligated into SpeI/NotI digested and dephosphorylated pCMV-BE4-Gam or pCMVAncBE4max vectors.

Cell Culture: All media component were purchased from ThermoFisher Scientific (Waltham, MA, USA) unless indicated otherwise. HEK293 cells were cultured on poly-L-ornithine (4 μg/ml; Sigma Aldrich, St Louis MO, USA) coated plates in the following media: 1× high glucose Dulbecco's modified Eagle's medium (DMEM), 10% (v/v) fetal bovine serum, 1% (v/v) L-glutamine penicillin/streptomycin. Culture medium changed was every other day and cells were passaged with Accutase (ThermoFisher) every 5 days. HPSCs were cultured on 12-well tissue culture plates coated with Matrigel™ (BD Biosciences, San Jose, CA, USA) in Essential 8™ Medium (E8) (ThermoFisher). HPSCs were cultured in mTESR1 medium (STEM CELL Technologies). Culture medium was changed every day and cells were passaged with Accutase every 4-5 days. After passaging, the medium was supplemented with 5 μM Rho kinase inhibitor (ROCKi; Y-27632 [BioGems, Westlake Village, CA, USA]) for 24 hours to aid in single cell survival.

Isolation of Episomal DNA: After 48 hours following transfection, HEK293 cells were dissociated from the tissue plates with Accutase, washed twice with phosphate-buffered saline and resuspended in RNAse-A containing solution. Cells were then lysed via alkaline lysis and the resultant debris was precipitated via centrifugation at 1.2×10⁴×g for 10 min. Supernatant DNA was isolated by column DNA purification using the manufacture recommended protocol (Sigma-Aldrich: NA0160).

Generation of HEK293-BFP line: The HEK293T-BFP cell line was generated via homology independent targeted integration (HITI). Briefly, the BFP coding sequence was PCR amplified with primers adding EcoRI restriction enzyme digestion sites. The resultant PCR product was EcoRI digested, phosphorylated and ligated into an EcoRI/Smal digested vector containing an EF1α promoter, puromycin resistance cassette and HITI protospacer sequence (pEF-BFP-PuroR). The pEFBFP-PuroR vector was co-transfected in HEK293s with pX330 (Addgene #42230) and a custom sgRNA vector (pHSG (C1ORF228)-1C3) targeting the C1ORF228 locus. Transfections were conducted in a 24-well plate with 300 ng pX330, 400 ng pEF-BFP-PuroR, 50 ng sgRNA vector, 1.5 μl Lipofectamine 3000 (ThermoFisher Scientific) and 1 μl P3000 transfection reagent. Cells were passaged at 72 hours post-transfection into a single well of a 6-well plate and selected with 0.5 μg/ml puromycin for 2 weeks.

### Results

### BFP-to-GFP Conversion Allows for Detection of Base-Editing Activity

To establish that BFP to GFP conversion could be used as the basis for an assay to detect genomic base editing, we utilized a BFP mutant that converts to a GFP upon a C-to-T nucleotide conversion (FIG. 1A). Briefly, this BFP mutant (BFPH66) contains a histidine at the 66th amino acid position encoded by a ‘CAC’ codon. The C-to-T conversion of that codon to ‘TAC’ or ‘TAT’ will result in an amino acid change from a histidine to a tyrosine. In turn, this amino acid change will cause a shift of the emission spectra of the resultant protein generating a GFP variant (GFP Y66). Because the optimal nucleotide base editing window is typically 12-18 nt upstream from the PAM, we also placed a S. pyogenes Cas9 PAM ‘NGG’ in a position that would enable base editing to occur at the target ‘CAC’ codon. To verify the utility of this fluorescent protein to report on base editing activity, we cloned the BFP coding sequence into a vector with a human EF1α promoter to drive expression (pEF-BFP; FIG. 1B). In addition, we designed a sgRNA vector [sg(BG)] that would direct the base editing machinery to the target ‘CAC’ codon resulting in a C-to-T conversion and the subsequent amino acid change of histidine to tyrosine at the 66th amino acid position (FIG. 1A). HEK293 cells were co-transfected with pEF-BFP, a base editing vector (pCMV-BE4-Gam) and sg(BG) or a control non-targeting sgRNA [sg(NT)]. Fluorescent microscopy (FIG. 1C) and flow cytometry (FIG. 1D) revealed that targeting pEF-BFP with sg(BG) resulted in the generation of BFP GFP double positive cells. However, targeting pEF-BFP with sg(NT) did not result in the generation of any BFP GFP positive cells. To confirm GFP expression was a consequence of direct editing of the target codon in pEF-BFP, we implemented a strategy to isolate and detect editing of episomal DNA after transfection (v 1E). Sanger sequencing of isolated pEF-BFP DNA established that editing had occurred at the target ‘CAC’ codon in pEF-BFP resulting in a change to ‘TAC’ or ‘TAT’ reflected in the GFP emission (FIG. 1F). Overall, these results confirm that the GFP-to-BFP conversion corresponds to C-to-T conversion at targeted base editing sites.

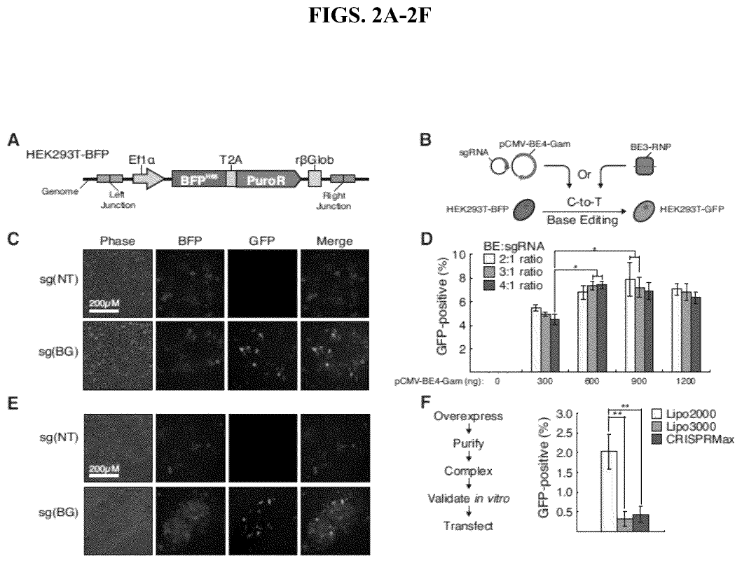

Next, we wanted to establish that the BFP-to-GFP conversion would correlate with base-editing efficiency at a chromosomal locus. To that end, we employed a HEK293 cell line (herein referred to as HEK293-BFP) in which BFPH66 was stably integrated into a known genomic location (C1ORF228; FIG. 2A). We then used this line to enable the analysis of the efficiency of base editing genomic loci (FIG. 2B). To first assess plasmid-based base editing, we co-transfected pCMV-BE4-Gam and sg(BG) plasmid DNA in HEK293-BFP cells. Targeting with sg(BG), but not sg(NT), resulted in generation of detectable GFP+ cells, indicating successful base editing at the targeted genomic loci (FIG. 2C). Moreover, we were able to use this assay to systematically evaluate genomic base editing efficiencies using a range of pCMV-BE4-Gam plasmid amounts at varying ratios with the sg(BG) vector (FIG. 2D). This analysis revealed that base editing plasmid concentration and base editor to sgRNA ratios could enhance genomic base editing efficiencies approximately 2-fold. Because ribonucleo-protein (RNP) complex-based strategies have been previously shown as an attractive alternative to plasmid-based Cas9 genome engineering, we also utilized BFP-to-GFP conversion as an assay to optimize RNP-driven base editing. As such, we generated RNPs through the in vitro complexing of purified base editing protein with sg(BG) or sg(NT) (FIG. 2E). Our initial analysis revealed that RNP delivery using the same transfection reagent that was used for plasmid delivery of the base editor (i.e., Lipofectamine™ 3000) did not result in substantial BFP-to-GFP conversion (FIG. 2F). In turn, we utilized BFP-to-GFP to evaluate various commercially available transfection reagents to optimize RNP delivery for base editing applications. From this analysis, we were able to determine that Lipofectamine™ 2000 allowed for a 4-fold increase in genomic base editing efficiency compared to other commercially available reagents such as Lipofectamine™ and CRISPRMAX (FIG. 2F). Despite this, RNP-driven delivery was about 4-fold less efficient in genomic base editing compared to plasmid delivery. Thus, for the remainder of this example we proceeded with plasmid delivery of base editors. Nonetheless, this collective data demonstrates that BFP-to-GFP conversion correlates to base editing efficiency at genomic loci. Moreover, this approach allows for the facile and systematic optimization of base editing in human cells using plasmid- and RNP-based approaches.

### Development of Transient Reporter for Editing Enrichment (TREE) to Identify and Efficiently Isolate Base-Edited Cell Populations

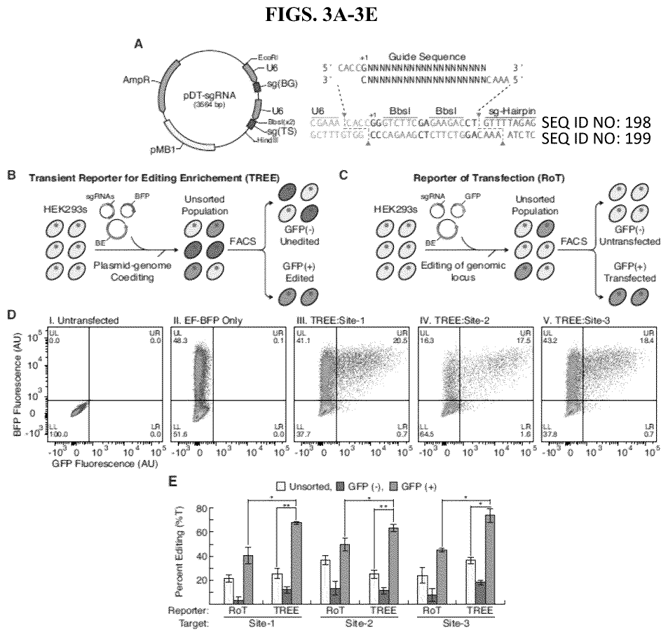

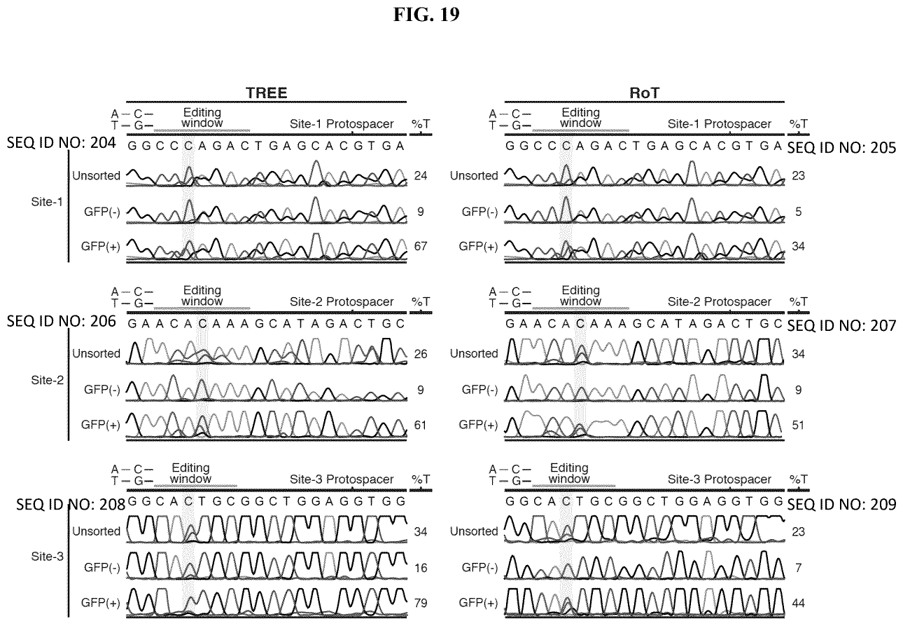

Conventional base editing approaches that use reporters of transfection (herein abbreviated as RoT) only report on the efficiency of plasmid delivery to a cell but not directly on the efficiency of base editing within these cells. As such, it was hypothesized that we BFP-to-GFP conversion, which directly correlates to base editing activity within a cell, could be employed as a TREE to allow for the identification and enrichment of cells in which targeted genomic base editing had occurred. To facilitate this, we engineered a dual-targeting sgRNA (pDT-sgRNA) vector that contains both sg(BG) and a sgRNA for a genomic target site [sg(TS)] (FIG. 3A). Moreover, the pDT-sgRNA vector was designed to allow for the facile cloning of new target sites via BbsI restriction enzyme digestion and ligation of sg(TS) oligonucleotides (FIG. 3A). Accordingly, we designed pDT-sgRNA vectors with sequences targeting three genomic locations (Sites 1-3). To utilize TREE for enrichment of cells that have been edited at specific loci, we co-transfected these pDT-sgRNA vectors with pEF-BFP and pCMV-BE4-Gam into HEK293 cells using the optimized base editing parameters identified using the BFP-to-GFP conversion assay (FIG. 3B). Flow cytometry was then used to isolate GFP-positive and GFP-negative cells. For comparison, we used a conventional ROT as a strategy to enrich for edited cell populations (FIG. 3C). Specifically, after co-transfecting HEK293 cells with pEF-GFP and sg(TS) plasmids, we used flow cytometry to sort for GFP-positive and -negative cell populations. Flow cytometry analysis of cells in which TREE was applied confirmed the presence of BFP and GFP-positive cell populations indicative of active base editing (FIG. 3D). Importantly, in these cell populations there was also a significant percentage of cells that were BFP-positive but GFP-negative, suggesting that isolating cell populations exclusively based upon a reporter of transfection would significantly limit the enrichment of edited cells. To confirm this, we performed Sanger sequencing of the targeted genomic sites in GFP-positive, GFP-negative and unsorted cell populations isolated from TREE and RoT approaches (FIG. 3E and FIG. 19). As expected, GFP-positive cells isolated using both TREE- and RoT-based strategies were enriched for edited cells when com-pared to GFP-negative and unsorted cell populations. We found that base editing efficiency at these three target loci in HEK293 cells using RoT-based approaches was similar to those reported previously (Table 11). Importantly, this analysis also revealed across all three targeted sites that GFP-positive cells isolated via TREE had a statistically significant higher frequency of base editing than GFP-positive cells isolated using traditional ROT approaches.

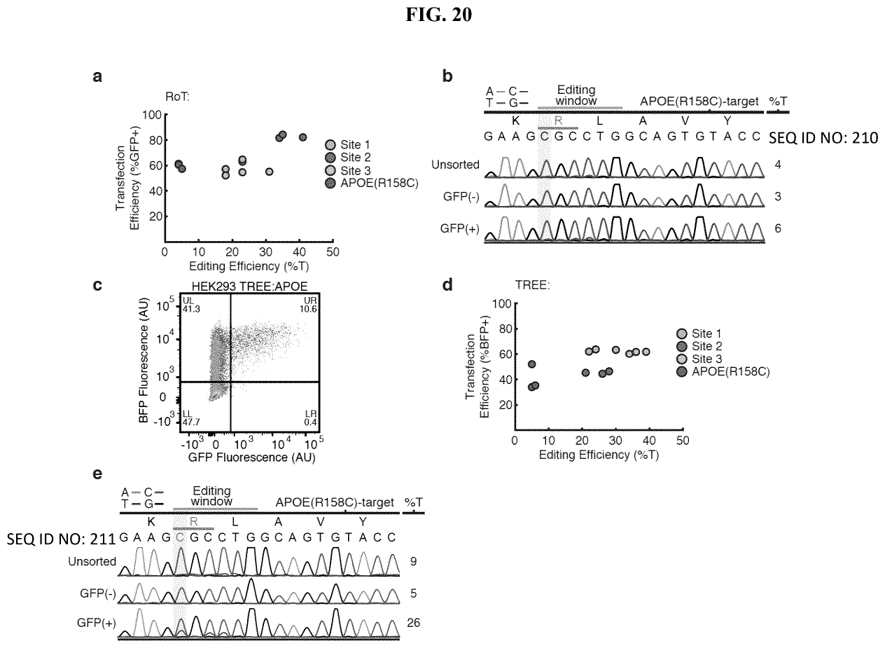

Because of the success of targeting these loci, it was investigated if TREE could be utilized to target additional genomic sites that display very low editing efficiency when traditional RoT approaches are applied. One such example is the APOE locus, a well-established risk factor associated with altered probability of sporadic Alzheimer's disease onset. Human APOE has three major isoforms, ApoE2, ApoE3 and ApoE4, which differ by two amino acid substitutions at positions 112 and 158 in exon 4-ApoE2 (Cys112, Cys158), ApoE3 (Cys112, Arg158), ApoE4 (Arg112, Arg158). Attempts to use base editing to convert ApoE3 to ApoE2 by targeting the APOE(R158) locus revealed undetectable levels of editing in unsorted cell populations despite similar transfection efficiencies when other genomic sites (Sites 1-3) were targeted (FIG. 20). In addition, our attempts to use ROT-based methods in HEK293 cells to convert ApoE3 to ApoE2 by targeting the APOE(R158) locus revealed very low levels of editing in GFP+ isolated cells (FIG. 20B), further establishing the APOE(R158) locus as recalcitrant to genomic editing. Then, TREE-based methods were used to edit this same loci in HEK293 cells by co-transfecting pEF-BFP, pCMV-BE4-Gam and pDT-sgRNA with a sg(TS) targeting the APOE(R158) locus. As expected, flow cytometry analysis demonstrated that the transfection efficiency when TREE was used to target the APOE(R158) locus was similar to when TREE was used to target other genomic sites (FIG. 20C). In addition, despite these similarities in transfection efficiencies, there was no detectable editing in the unsorted cell populations using TREE to target the APOE(R158) locus, thereby confirming the difficulty in editing this genomic location (FIG. 20D). However, unlike in GFP-positive isolated using ROT methods, GFP-positive cells purified using TREE methods displayed a high level of base editing at the APOE(R158) locus (FIG. 20E). Together, these results demonstrate that TREE can not only provide for a higher level of enrichment of base-edited cell populations compared to conventional RoT strategies but also can allow for isolation of base-edited cells at genomic loci that were not previously achievable with traditional ROT approaches.

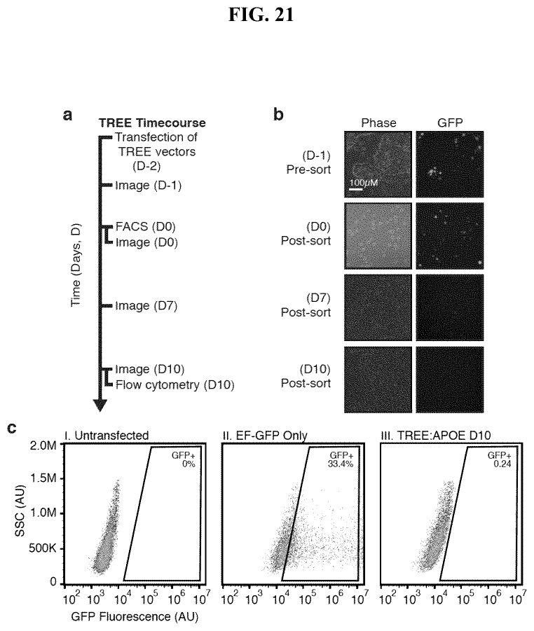

At last, we wanted to confirm that the fluorescent signal associated with cells isolated by TREE was transient. To that end, we measured the long-term fluorescence of GFP-positive cells purified after TREE-based editing (FIG. 21A). Notably, analysis of these cells by fluorescent microscopy (FIG. 21B) and flow cytometry (FIG. 21C) revealed no long-term detectable GFP signal, verifying that the TREE fluorescent output is indeed transient in nature.

### Multiplex Base-Editing Using TREE

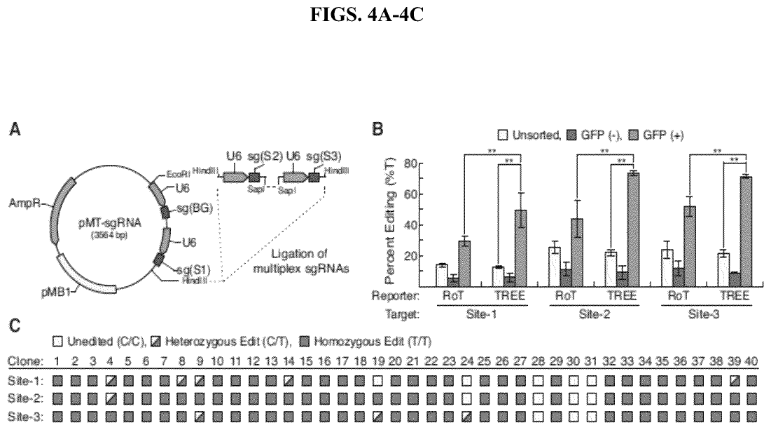

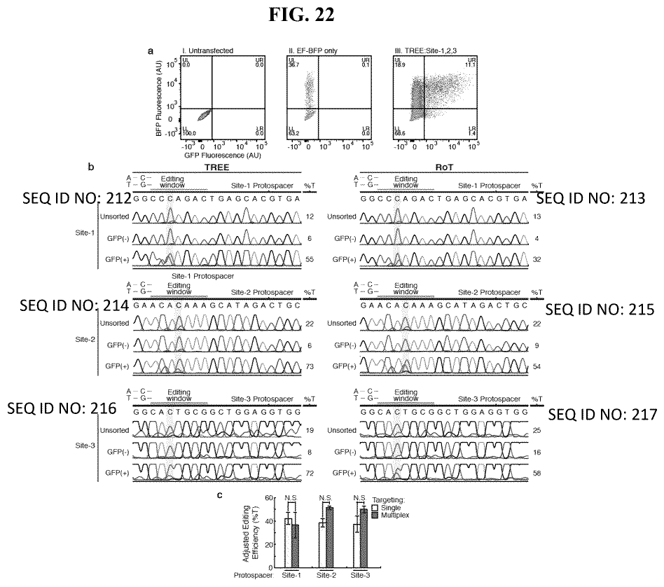

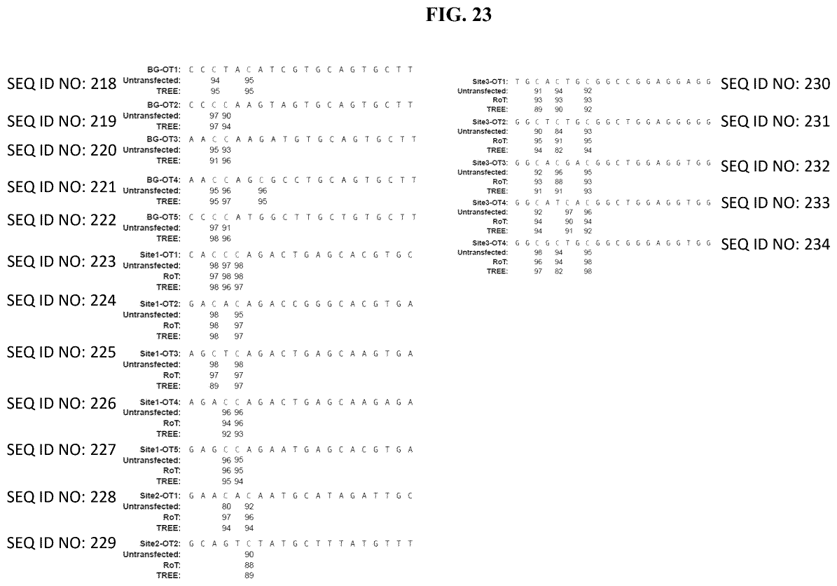

It was further investigated whether TREE could be utilized in con-junction with multiplexed genome engineering strategies. To accomplish this, we generated a multi-targeted vector (pMT-sgRNA) that contains sg(BG) as well as sgRNA for genomic targets Sites 1-3 (FIG. 4A). In a similar manner to when TREE was employed to target a single locus, we utilized TREE to simultaneously target multiple genomic sites by co-transfecting HEK293 cells with pMT-sgRNA, pEF-BFP and pCMV-BE4-Gam. In parallel, we used a RoT-based approach by co-transfecting HEK293 cells with pMT-sgRNA, pEF-GFP and pCMV-BE4-Gam. After 48 h, GFP-negative and GFP-positive cells were isolated using flow cytometry (FIG. 22A). Along similar lines to single locus targeting, Sanger sequencing of the multiplex targeted genomic sites in GFP-positive cell populations isolated from TREE and RoT approaches revealed that TREE allowed for statistically significant higher frequency of base editing than ROT approaches (FIG. 4B and FIG. 22B). Importantly, this analysis revealed that there was no statistically significant difference in editing efficiency when TREE was used to target these sites individually or a multiplexed manner (FIG. 22C). Finally, we wanted to determine if TREE increased the likelihood of C-to-T conversions at off-target loci. Therefore, in GFP-positive cell populations isolated from TREE and ROT approaches we PCR-amplified and Sanger sequenced the top predicted off-target loci for the sgRNA sequences used for multiplexed editing. Overall, quantification of the Sanger chromatographs by EditR revealed no observable C-to-T conversions at these off-target loci in either GFP-positive cells isolated with TREE- or RoT-based strategies when compared to that of untransfected cells (FIG. 23).

Sanger sequencing that was performed on bulk sorted GFP-positive cells suggested that multiplex editing in conjunction with TREE could result in multiplexed editing in the same cell. To confirm that this indeed occurred, we again used our multi-targeting vector (pMT-sgRNA) in conjunction with TREE to simultaneously target genomic Sites 1-3 in HEK293 cells. We then sorted single GFP-positive cells into a 96-well plate. After expansion, Sanger sequencing of the multiplexed genomic sites was performed on a total of 40 clones. This analysis revealed that 36 out of the 40 clones had base editing at more than one genomic site (FIG. 4C). Remarkably, this analysis revealed that almost 80% of the isolated clones (31 out of 40) had biallelic conversions at all three genomic loci.

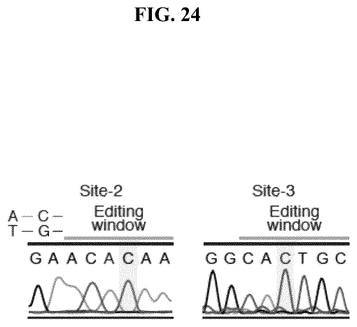

One of the caveats of all base-editing approaches, regardless if RoT- or TREE-based enrichment strategies are employed, is that base editors can potentially edit non-target Cs that are located in an 6 nt window (termed the editing window) within the protospacer. As a consequence, this could potentially limit the application of base editing approaches in which conversion of non-target Cs result in a non-silent mutation or other phenotypic changes. To that end, we wanted to determine if any of our clones contained edits exclusively at the target C and not any other Cs within the editing window. Indeed, we identified a number of clones in which at genomic Site 2 and Site 3 modification only occurred at the target C (FIG. 24). Interestingly, we did not identify any clones in which at genomic Site 1 such exclusive modification of the target C occurred. We speculate that because another C occurs immediately adjacent to this target C, that such exclusive modification will require the use of recently published site-specific editors that allow for single nucleotide changes free from off-targeting conversions within the editing window.

TREE Allows for Highly Efficient Editing in Human Pluripotent Stem Cells (hPSCs)

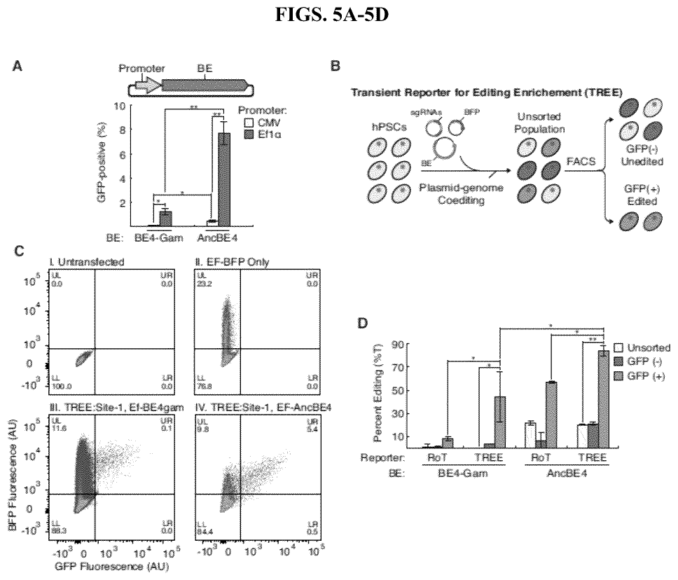

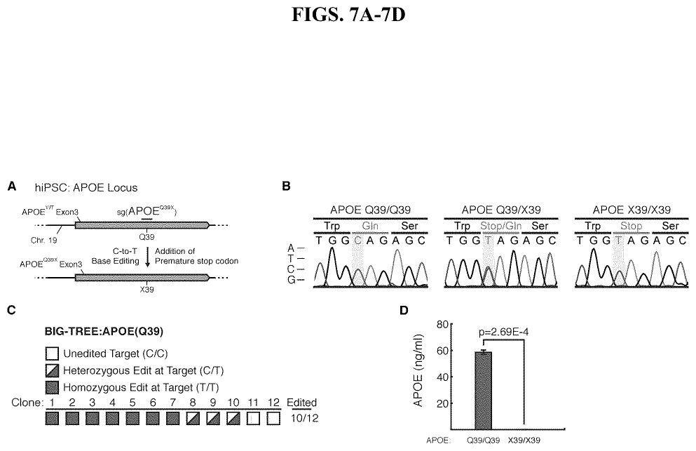

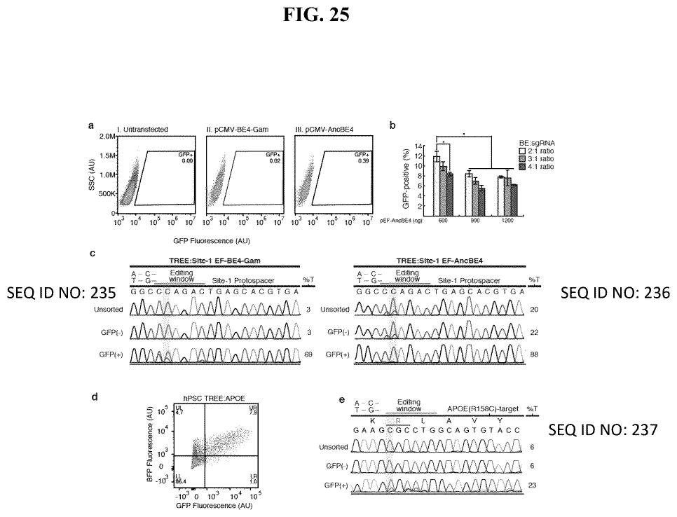

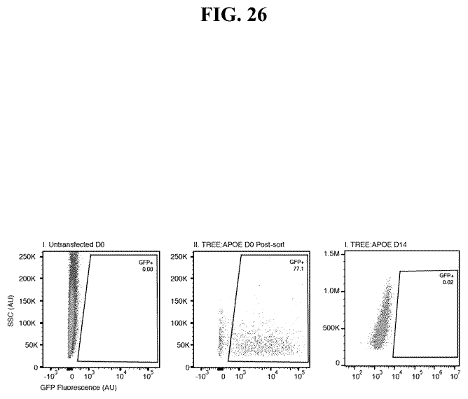

Single base pair modification in hPSCs viaCRISPR Cas9-induced DSB followed by HDR suffers from low efficiencies. In addition, genomic modification of hPSCs using deaminase-based DNA base editor has yet to be reported. Therefore, we wanted to investigate if TREE could be utilized to efficiently edit specific loci in hPSCs. Hence, we co-transfected pEF-BFP and pCMV-BE4-Gam into hPSCs using a transfection reagent (Lipofectamine™ Stem) that had been previously used by others for the efficient delivery of Cas9-related plasmids to hPSCs. Surprisingly, we did not observe many GFP-positive cells in these cell populations (FIG. 5A and FIG. 25A). As such, we performed similar experiments in which we employed a more recently published, higher efficiency base editor, AncBE4max (herein referred to AncBE4). Briefly, AncBE4 is an improved version of BE4 that has been codon optimized for expression and contains an ancestral reconstructed deaminase to increase base editing efficiency at tar-get loci. Nonetheless, similar to when BE4-Gam was utilized, we observed very few GFP-positive cells when An-cBE4 was used (FIG. 25A). Because previous reports have suggested that the CMV promoter is inefficient for transgene expression in pluripotent stem cells, we replaced the promoter driving base editor expression with EF1α. When hPSCs were co-transfected with pEF-BE4-Gam or pEF-AncBE4 as well as pEF-BFP and sg(BG), a significant number of GFP-positive cells were observed (FIG. 5A). Using the pEF-AncBE4 vector, we also developed editing efficiency in hPSCs by using a range of base editor amount at varying ratio with sg(BG) (FIG. 25B). Similar to our experiments with HEK293 cells, this analysis revealed that base editing efficiencies were significantly affected by these parameters. Interestingly, the most optimal parameters in hPSCs differed from those identified in HEK293 cells (FIG. 2D) highlighting the utility of this assay to evaluate these variables. Using these base editing vector designs, we applied TREE to target a genomic loci in hPSCs by co-transfecting pEF-BE4-Gam pEF-AncBE4, pEF-BFP and pDT-sgRNA (with a sg(TS) targeting site 1) (FIG. 5B). In turn, flow cytometry was used to isolate GFP-positive and -negative cell populations (FIG. 5C). Subsequently, Sanger sequencing was performed on the targeted genomic site in GFP-positive, GFP-negative and unsorted cell populations isolated from TREE and ROT approaches in which pEF-BE4-Gam and pEF-AncBE4 was used (FIG. 25C). This analysis demonstrated that GFP-positive hPSCs isolated via TREE had a statistically significant higher frequency of base editing than GFP-positive hPSCs isolated using traditional ROT approaches (FIG. 5D). In addition, TREE employed with the pEF-AncBE4 vector allowed for the efficient modification of the difficult to edit APOE(R158) locus (FIG. 7D-S7E).

Similar to our work with HEK293 cells, we wanted to confirm that the fluorescent output of TREE was transient in nature. In that regard, we measured the fluorescence of GFP-positive hPSCs isolated after TREE-based editing. Flow cytometry analysis revealed that after 2 weeks of culture there was no detectable GFP signal (FIG. 26), demonstrating that the fluorescent signal associated with hPSCs purified by TREE was transient.

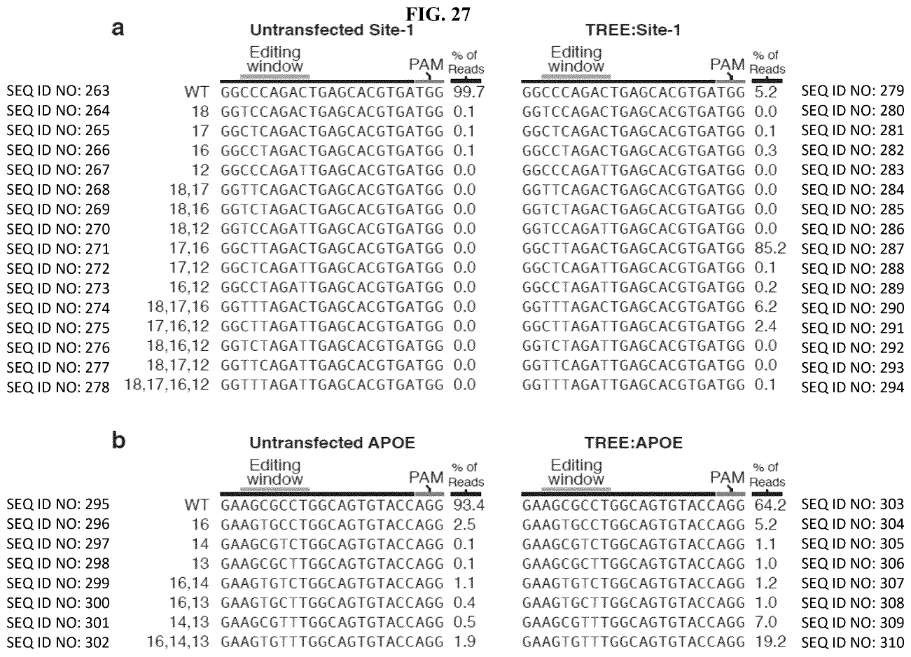

Collectively, although this data demonstrates that TREE can be utilized for the efficient base editing of hPSCs, one of the caveats of all base editing approaches is the C-to-T conversion of non-target Cs within the editing window. Indeed, the Sanger sequencing analysis of GFP-positive populations isolated from TREE revealed editing of such non-target Cs when either Site 1 (FIG. 25C) or the APOE(R158) (FIG. 25E) locus was targeted in hPSCs. As such, to determine whether TREE allowed allelic outcomes in which targeting only occurred at the desired C, we performed NGS of PCR amplicons of Site 1 and APOE(R158) in GFP-positive cells purified using TREE. This analysis revealed at both these loci a very modest number of allelic outcomes in which base editing occurred exclusively at the target C, free from con-founding C-to-T conversions at other sites within the targeting window (FIG. 27). Instead, the most common editing outcome was one in which the majority of the Cs in the editing window were converted to Ts (FIG. 27). This suggests that for future applications which require a higher percentage of allelic outcomes where editing occurs only at the target C the use of recently published base editors that have a narrower editing window will be required. Nonetheless, this collective data demonstrates the broad utility of TREE to allow for the efficient editing in hPSCs.

### Discussion

Since the first deaminase base editor was developed by Komor et al., multiple additional base-editing technologies have been rapidly developed with various endonucleases, deaminases, targeting windows and PAM specificities. Application of these emerging base editors to new cell types requires a slow, iterative process in which various base editing parameters are tested and editing efficiency is assessed through downstream sequencing methods. Additionally, as demonstrated herein, transfection efficiency does not precisely correlate with editing efficiency, so reporters of transfection do not provide accurate information about the efficacy of various base editing strategies. Here, it is described how BFP-to-GFP conversion and TREE can be utilized to rapidly optimize various factors that influence base editing efficiency, including base editor plasmid concentration and design as well as base editor to sgRNA ratios. In fact, these data show that these parameters are cell line-specific, demonstrating the advantage of TREE to allow for the high-throughput evaluation of base editing approaches. In the future, TREE may be used in the context of high-throughput screening to identify small molecules to further enhance base editing efficiency in a manner similar to that which has been previously achieved with CRISPR-mediated HDR approaches.

It has been shown that CRISPR Cas9 genome engineering is compatible with a variety of delivery methods (e.g., lipid-mediated transfection, electroporation) and expression systems (e.g., plasmid DNA, Cas9-gRNA ribonucleo-protein complexes [RNP]), each with advantages and dis-advantages that have been reviewed extensively elsewhere. In this example, we employed lipid-based delivery reagents that have been previously employed by others for the CRISPR Cas9-based editing of HEK293 cells (Lipofectamine 3000) and hPSCs (Lipofectamine Stem). Given TREE's ease of use and readily detectable fluorescent output we anticipate that TREE can be employed with whatever transfection method that is preferred by the end user. For instance, the data presented here demonstrated that the TREE base editing assay was compatible with both plasmid and RNP approaches. Although we observed that the overall genomic base editing efficiency of RNP-based expression was lower than that of lipid-based expression, we provide proof-of-principle that TREE can be employed in future applications where the advantages of RNPs are desirable.

One potential limitation of the use of the plasmid DNA expression systems in the context of TREE approaches is random integration of all or part of the plasmid DNA into the genome of targeted cells. It should be noted that it has been reported by others that the stable integration of circular plasmid DNA into the host genome is infrequent, especially for cells such as hPSCs where it has been reported on the order of 1 per 1×10⁵ cells. Indeed, as it relates to potential integration of the pEF-BFP plasmid, we demonstrate that the fluorescent output of TREE is transient in both HEK293 cells and hPSCs, suggesting that this plasmid does not integrate into the genome. As it relates to the integration of the base editing and sgRNA plasmids, it has been shown by others in CRISPR Cas9 genome engineering that the Cas9 and sgRNA plasmids can be integrated at on- and off-target sites. However, we speculate that because base editors do not introduce DSBs the integration of these plasmids into the genome would be infrequent. In fact, we did not observe any integration of these plasmids when Sanger sequencing or NGS was performed at the on- or off-target sites. Moving forward, undesirable insertions of plasmid DNA sequences at target sites can be detected using PCR-based methods followed by Sanger sequencing or NGS of the resultant amplicons. On the other hand, similar insertions at off-target or random genomic sites are difficult to detect and will require the use of more comprehensive techniques such as whole genome sequencing.

Human cell models are critical for elucidating the mechanisms of disease progression as well as identifying and testing potential therapeutic interventions. Because a high percentage of human diseases are due to single nucleotide poly-morphisms (SNPs), base editors can allow for the precise engineering of in vitro models of human disease. Here we provide proof-of-principle that TREE can be employed to edit disease-relevant loci. Specifically, we demonstrate that TREE enables for the enrichment of cells that had been edited at the APOE(R158) locus, a gene associated with altered risk of Alzheimer's disease onset. Notably, conventional RoT-based methods did not allow for significant enrichment of edited cells at this same refractory locus. In addition, because many human diseases are multigenetic disorders that are a result of complex gene interactions, we also investigated the ability of TREE to be utilized in multiplexed genome engineering applications. By using a multi-targeted vector, we demonstrated that compared to RoT-based methods TREE resulted in a significantly higher level of cells enriched for simultaneous editing at multiple independent loci. In fact, we demonstrated that through analysis of single cell clones that 90% of the clones had simultaneous base editing at more than one genomic site and almost 80% of the clones had biallelic conversions at all three targeted loci. In this vein, TREE provides a highly efficient method for generating cell-based models of multigenic diseases.

Many immortalized cell lines, such as HEK293s, are aneuploid with unknown mutations and dosage at key disease-relevant genes. Alternatively, hPSCs, which have a normal euploid karyotype and the potential to differentiate into all cell types of the mature adult body, represent an attractive alternative to immortalized cell lines for disease modeling and drug screening applications. In particular, the ability to use gene editing technologies to generate isogenic hPSC lines that differ only with respect to disease mutations has great potential as it relates to precisely defining genotype to phenotype relationships. The RNA-guided CRISPR-Cas9 system has the potential to allow for precise genetic modifications in hPSCs through the introduction of site-specific DSBs. Although previous reports demonstrate that introduction of DSB via CRISPR Cas9 significantly improves the ability to obtain knock out cell lines from hPSCs by the NHEJ pathway, single base modification using CRISPR Cas9-induced DSB followed by HDR is extremely inefficient (1-2% of sequenced colonies in which one allele is targeted and 1% where both alleles are targeted. Recently, it has been reported that co-delivery of Cas9, sgRNA, and a puromycin selection cassette followed by transient puromycin selection can increase the HDR-mediated genome engineering in hPSCs However, these strategies rely on the introduction of DSBs, which in pluripotent stem cells can lead to large deletions and complex chromosomal rearrangements, significant cytotoxicity and increased acquisition of p53 mutations. In addition, it has been shown that the use of antibiotic selection, even in a transient manner, may lead to the se-lection of hPSCs, with chromosomal abnormalities. Yet, to our knowledge, base editors, which do not have these same limitations as CRISPR Cas9-induced DSB followed by HDR, have not previously been used with hPSCs. In fact, our initial attempts to apply base editors in the context of both RoT- and TREE-based approaches with hPSCs did not allow for observable modification of target loci. Instead, by replacing the standard CMV promoter in the base editing plasmids with an EF1α promoter, we were able to achieve modification of genomic sites using both RoT- and TREE-centered methods. However, TREE allowed for significantly higher enrichment of edited hPSCs when com-pared to RoT isolation strategies. We contend that the use of TREE with hPSCs will significantly advance the use of these cells in disease modeling, drug screening, and cell-based therapies.

Despite their tremendous potential in a variety of downstream applications, base editing approaches have a few of caveats that should be noted, regardless of whether RoT- or TREE-based enrichment strategies are employed. First, as is the case with all Cas9-directed genome editing approaches, is the potential for genome modification at off-target loci. In this work, GFP-positive cells isolated via TREE did not display untargeted C-to-T conversions at the off-target genomic loci examined. Recently, it has been reported that base editors can induce site-specific inosine formation on RNA. Accordingly, in the future, the effect of TREE-based approaches on unwanted RNA modifications should be examined. Another limitation of base editing methods is modification of additional C nucleotides that are in close proximity to the target C. In fact, some base editors can cause C-to-T conversions at any Cs in up to a 9-nt window within the protospacer. Such C-to-T modifications could be especially problematic if they result in amino acid alterations during translation, induce epigenetic changes or cause other phenotypic changes in targeted cells. To that end, through clonal isolation and next generation sequencing (NGS) analysis we identified that such exclusive modifications of the target C were achieved in both edited HEK293 cells or hPSCs that were enriched using TREE-based methods. It should be noted, though, that at genomic Site-1, where a C lies adjacent to the target C, allelic outcomes in which modification only occurred at the target C were rare events. Moving forward, modified base editors that have a narrow editing window could be easily employed with TREE to target such genomic loci that contain multiple Cs in close proximity to the target C.

In summary, these data demonstrate that TREE allows not only for the optimization of base editing strategies in the context of a variety of cell types and genomic locations but also the enrichment of cell populations to be utilized in variety of downstream applications. In particular, with the rate at which the genome editing field has been progressing over the past few years, TREE is a readily adoptable method that will expedite and improve tractability of single-nucleotide genome engineering methods.

### Example 2—Producing Base-Edited Isogenic Cell Lines Using a Transient Reporter for Editing Enrichment (BIG-TREE)

Current CRISPR-targeted single-nucleotide modifications and subsequent isogenic cell line generation in human pluripotent stem cells (hPSCs) require the introduction of deleterious double-stranded DNA breaks followed by inefficient homology-directed repair (HDR). This section describes the development of Cas9 deaminase base-editing technologies to co-target genomic loci and an episomal reporter to enable single-nucleotide genomic changes in hPSCs without HDR. Using this method, a base-edited isogenic hPSC line was generated using a transient reporter for editing enrichment (BIG-TREE) which allows for single-nucleotide editing efficiencies of >80% across multiple hPSC lines. Also described herein is use of BIG-TREE for efficient generation of loss-of-function hPSC lines via introduction of premature stop codons. Finally, BIG-TREE achieves efficient multiplex editing of hPSCs at several independent loci. These methods allow for the precise and efficient base editing of hPSCs for use in developmental biology, disease modeling, drug screening, and cell-based therapies.

### Materials and Methods

Cells and Culture Conditions: Cell lines, media compositions, and conditions for culture of hPSC and HEK293 are listed in the Supplemental Experimental Procedures (End of Example 2).

Plasmid Construction: All plasmids were constructed using conventional restriction enzyme-based molecular cloning techniques. For construction of the sgRNA plasmids, the sgRNA sequences listed in Table 12 were used. Additional details for molecular cloning and plasmid construction are provided in the Supplemental Experimental Procedures (End of Example 2).

sgRNA Sequences in TREE: Guide and Cas9 Handle

>sg(BG)
(SEQ ID NO: 3)
GACCCACGGCGTGCAGTGCTTGTTTTAGAGCTAGAAATAGCAAGTTAAAA
TAAGGCTAGTCCGTTATCAACTTGAAAAAGTGGCACCGAGTCGGTGCTTT
TTTGTTTT
>sg(NT)
(SEQ ID NO: 4)
GGGTCTTCGAGAAGACCTGTTTTAGAGCTAGAAATAGCAAGTTAAAATAA
GGCTAGTCCGTTATCAACTTGAAAAAGTGGCACCGAGTCGGTGCTTTTTT
GTTTT
>sg(Site-1)
(SEQ ID NO: 5)
GGCCCAGACTGAGCACGTGAGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(Site-2)
(SEQ ID NO: 6)
GAACACAAAGCATAGACTGCGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(Site-3)
(SEQ ID NO: 7)
GGCACTGCGGCTGGAGGTGGGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(APOE R158)
(SEQ ID NO: 8)
GAAGCGCCTGGCAGTGTACCGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(C1ORF228)
(SEQ ID NO: 9)
GTGCTGTTAGCACCCTGGAAAGTTTTAGAGCTAGAAATAGCAAGTTAAAA
TAAGGCTAGTCCGTTATCAACTTGAAAAAGTGGCACCGAGTCGGTGCTTT
TTTGTTTT
>sg(APOE Q39X)
(SEQ ID NO: 10)
GTGGCAGAGCGGCCAGCGCTGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(mCh1)
(SEQ ID NO: 11)
GCACCCAGACCGCCAAGCTGAGTTTTAGAGCTAGAAATAGCAAGTTAAAA
TAAGGCTAGTCCGTTATCAACTTGAAAAAGTGGCACCGAGTCGGTGCTTT
TTTGTTTT
>sg(mCh2)
(SEQ ID NO: 12)
GACCCAGGACTCCTCCCTGCGTTTTAGAGCTAGAAATAGCAAGTTAAAAT
AAGGCTAGTCCGTTATCAACTTGAAAAAGTGGCACCGAGTCGGTGCTTTT
TTGTTTT
>sg(mCh3)
(SEQ ID NO: 13)
GCAAGCAGAGGCTGAAGCTGAGTTTTAGAGCTAGAAATAGCAAGTTAAAA
TAAGGCTAGTCCGTTATCAACTTGAAAAAGTGGCACCGAGTCGGTGCTTT
TTTGTTTT

hPSC Base Editing, Clonal Isolation, and Characterization: Methods for transfection of hPSCs, clonal isolation, and characterization via tri-lineage differentiation are described in the Supplemental Experimental Procedures (End of Example 2).

Genotyping and Sequence Analysis at Off- and On-Target Analysis: Genomic DNA was prepared from expanded clones using the DNeasy kit (QIAGEN). PCR was performed with the primers listed in Table 5 using the methods described in the Supplemental Experimental Procedures (End of Example 2).

Karyotype Analysis: For each cell line, cytogenetic analysis was performed (Cell Line Genetics) on 20 metaphase cells using standard protocols for G-banding.

Immunofluorescence: Detailed protocols for immunofluorescence and antibodies used are provided in the Supplemental Experimental Procedures (End of Example 2).

HEK293 Transfections: Methods for transfection of HEK293s and Sanger sequencing of resultant populations are described in the Supplemental Experimental Procedures (End of Example 2).

Fluorescence Microscopy: Fluorescent imaging was performed on a Nikon Ti-Eclipse inverted microscope using the filters and acquisition settings described in the Supplemental Experimental Procedures (End of Example 2).

Flow Cytometry: Cells were dissociated with Accutase for 10 minutes at 37° C., triturated, and passed through a 40 mm cell strainer. Cells were then washed twice with flow cytometry buffer (BD Biosciences) and resuspended at a maximum concentration of 5×10⁶ cells per 100 mL. Flow cytometry analysis was performed on an Attune NxT (Thermo Fisher Scientific). Flow cytometry files were analyzed using FlowJo (FlowJo LLC, Ashland, OR, USA).

Apolipoprotein E ELISA: Cells were seeded in a 6-well plate at a density of 3×10⁵ cells per well. Medium was changed every 24 hours (h). On day 3, 24-h conditioned medium was collected, and ApoE levels in the medium were measured with the Human APOE(AD2) ELISA Kit (Thermo Scientific).

Statistical Analysis: Unless otherwise noted, all data are displayed as means #SD.

### Results

Highly Efficient Generation of Clonal Isogenic hPSC Lines Using BIG-TREE

It was previously demonstrated that the efficiency at which a base editor is delivered to a cell does not precisely correlate with editing efficiency at a genomic locus. To overcome this limitation, an assay was developed, termed transient reporter for editing enrichment (TREE) (Standage-Beier et al., 2019). TREE utilizes a BFP variant that converts to a GFP upon a C-to-T nucleotide change (Standage-Beier et al., 2019). More specifically, this BFP mutant contains a histidine at the 66th amino acid position encoded by a “CAC” codon. The C-to-T conversion of that codon to a “TAC” or “TAT” will cause an amino acid change from a histidine to a tyrosine as well as a shift in the emission spectra of the modified protein resulting in a GFP variant. Thus, co-transfection of cells with this BFP construct (pEF-BFP), a base editor (pEF-AncBE4max), and a single guide RNA (sgRNA) targeting the “CAC” codon, sg(BG), will result in a BFP-to-GFP conversion in which the base editor machinery is present and actively functioning. In addition, we found that this BFP-to-GFP conversion was highly predictive of the likelihood of base editing at genomic loci within the same cell that had been transfected with a sgRNA for a genomic target site, sg(TS).

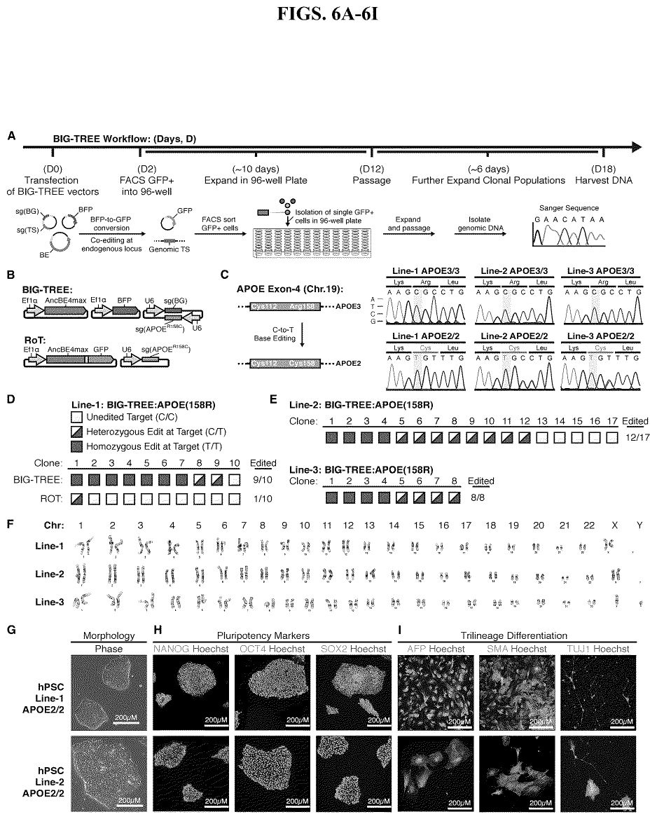

This section describes efforts to extend this work to develop a rapid and efficient resource that uses TREE as the basis for the generation of clonal isogenic hPSC lines, termed base-edited isogenic hPSC line generation using a transient reporter for editing enrichment (BIG-TREE) (FIG. 6A). As proof-of-principle, we aimed to edit the APOE locus, a risk factor associated with altered probability of sporadic Alzheimer disease (AD) onset (Hauser and Ryan, 2013). Human APOE has three common isoforms that differ from each other by two amino acids at position 112 and 158 (APOE2=Cys112, Cys158; APOE3=Cys112, Arg158; APOE4=Arg112, Arg158). To this end, we transfected a non-demented control hPSC line (herein referred to as hPSC line 1) that has an APOE3/E3 genotype with pEF-BFP, pEF-AncBE4max, and a dual-targeting sgRNA (pDT-sgRNA) vector that contains both sg(BG) and a sgRNA for the APOE(R158) locus (FIG. 6B, top). Consequently, successful targeting of the APOE(R158) locus would result in C-to-T conversion that would cause a change from an APOE3 genotype (R158) to an APO E2 genotype (C158) (FIG. 6C). At 48 hours post transfection, fluorescent activated cell sorting (FACS) was used to sort single GFP-positive cells into 96-well plates. Clonal lines were then passaged and expanded over the course of 18 days prior to detailed analysis. First, genomic DNA was isolated from ten clones and the target region of the APOE locus, APOE(R158), was subject to Sanger sequencing after PCR amplification (FIG. 6C). Remarkably, this analysis revealed that 90% of the clones isolated had been edited, with seven of the clones having a homozygous and two of the clones having a heterozygous edit at the APOE(R158) locus (FIG. 6D). For comparison, we used a more conventional reporter of transfection (RoT) approach in which this same hPSC line was transfected with a plasmid in which a GFP and the AncBE4max base editor are driven by the same promoter, connected by a P2A post-translational self-cleavage peptide tag (pEFAncBE4max-P2A-GFP), as well as the same sgRNA for the APOE locus (FIG. 6B, bottom). In a manner analogous to that described for the BIG-TREE-based approach, single GFP-positive cells were then sorted into 96-well plates, expanded, and subject to Sanger sequencing. Analysis of ten clonal lines revealed this traditional RoT-based approach was significantly less efficient with only a single clone displaying a heterozygous edit at the target APOE(R158) locus (FIG. 6D). Given the large variability that exists between individual hPSC lines (Ortmann and Vallier, 2017), we wanted to determine the robustness of BIG-TREE to efficiently generate isogenic pairs in other independent hPSC lines. In this vein, we employed BIG-TREE to target the APOE(R158 locus) in two hPSC lines derived from patients with familial AD (FAD) (herein referred to hPSC line 2 and hPSC 3). Analysis of single cell clones by Sanger sequencing (FIG. 6C) revealed that across all three hPSC lines tested, over 80% (33/41 clones examined) had an edit at the APOE(R158) locus, and greater than 50% of those edits were homozygous in nature (FIG. 6E). Importantly, we did not observe the presence of indels at the target site in any of clones examined. Finally, one of the limitations of base editor techniques, regardless if BIG-TREE strategies are employed, is that base editors can induce changes in the protospacer at a C other than the target C within the editing window-termed bystander editing (FIG. 21A). Indeed, with respect to generating isogenic lines at the APOE(R158) locus, editing at these bystander Cs was a common occurrence (FIG. 21B). In fact, only one of the clones analyzed (line 2, clone 5) had a heterozygous edit exclusively at the target C and no other Cs within the editing window. However, it should be noted that these bystander edits did not alter the amino acid sequence.

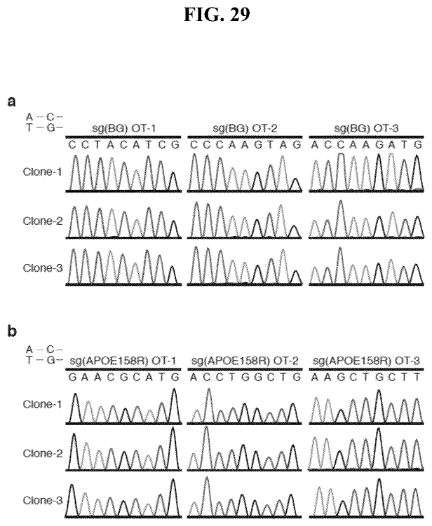

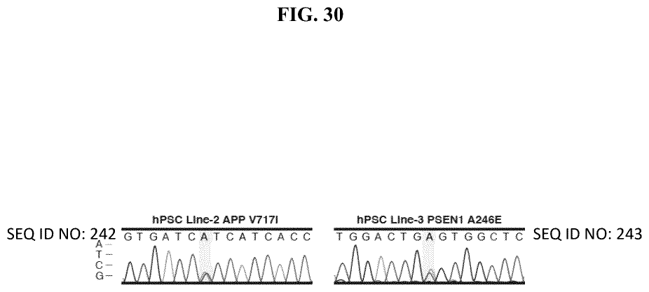

We performed detailed phenotypic analysis on representative biallelic edited clones from each hPSC line. Overall, these clones had a normal euploid karyotype (FIG. 6F), characteristic hPSC morphology (FIG. 1G), high expression of key pluripotency markers (FIG. 1H), and demonstrated tri-lineage differentiation potential (FIG. 61). In addition, off-target analysis was performed at the top predicted sites for sg(BG) as well as the sgRNA used to target the APOE(R158) locus. At all of the off-target sites analyzed, we did not observe any C-to-T conversions at these off-target loci (FIG. 29). Furthermore, indels were not identified at any of the off-target sites in clones analyzed. Finally, Sanger sequencing revealed that the AD-related mutations in the hPSC clones derived from the FAD lines were retained in the edited clones (FIG. 30). Taken together, this analysis reveals that TREE can be employed for the highly efficient generation of isogenic hPSCs across multiple independent cell lines.

BIG-TREE can be Utilized for the Engineering of Gene Knockout hPSC Lines

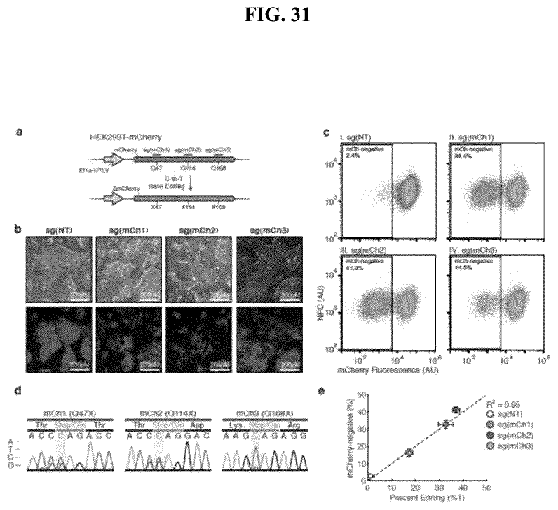

To date, engineering of hPSC loss-of-function lines using CRISPR-based approaches has involved the generation of Cas9-mediated DSBs followed by non-homologous end joining (NHEJ), which typically results in a frameshift mutation and introduction of a downstream premature stop codon. Because of the aforementioned caveats associated with such DSB-driven approaches, we wanted to determine if BIG-TREE could be utilized to generate gene knockout hPSC lines without the introduction of DSBs. Because base editors have not been utilized previously to generate loss-of-function in hPSCs, we first wanted to establish this proof-of-principle in HEK293 cells. First, to validate base editor targeted introduction of premature stop codons, we designed a series of sgRNAs targeting an mCherry cassette in an HEK293T line, which would lead to conversion of a “CAG” codon encoding for glutamine to a “TAG” stop codon (FIG. 31A). We observed loss of mCherry expression via fluorescent microscopy and flow cytometry when targeting with sgRNAs (FIGS. 31B and 31C). In addition, we confirmed the targeted addition of stop codons by Sanger sequencing (FIG. 31D). Finally, this analysis revealed that loss of mCherry fluorescent signal was a direct consequence of introduction of a premature stop codon introduced into the genomically integrated mCherry cassette (FIG. 31E). Next, we sought to employ BIG-TREE to introduce premature stop codons in hPSCs at a disease relevant locus. To this end, we transfected hPSC line 1 with pEF-BFP, pEF-AncBE4max, and a dual-targeting sgRNA (pDT-sgRNA) vector that contained both sg(BG) and a sgRNA for the glutamine residue at amino acid position 39 in exon 3 of the APOE locus. Successful targeting would result in conversion of the glutamine encoding “CAA” codon to a premature “TAA” stop codon (FIG. 7A). Similar to as previously described, we isolated clonal cell lines established from single GFP-positive sorted cells. Analysis of these clones by Sanger sequencing (FIG. 7B) revealed that more than 80% of the clones had a stop codon introduced at the target site with greater than 50% of the edited clones displaying a biallelic modification (FIG. 7C). Importantly, none of the clones analyzed had indels at the same target site. Lastly, to demonstrate that introduction of a premature stop codon in exon 3 results in functional loss of APOE, we measured the amount of APOE in the conditioned media secreted by unedited and edited cells using ELISA. Compared with the unedited wild-type (Q39/Q39) cells that secreted robust amounts of APOE, cells in which a premature stop codon had been introduced into both alleles (X39/X39) did not secrete any detectable levels of APOE(FIG. 7D). Collectively, these data show that BIG-TREE enables efficient generation of loss-of-function hPSC lines through the introduction of premature stop codons.

BIG-TREE Enables High-Frequency, Multiplex Base Editing in hPSCs

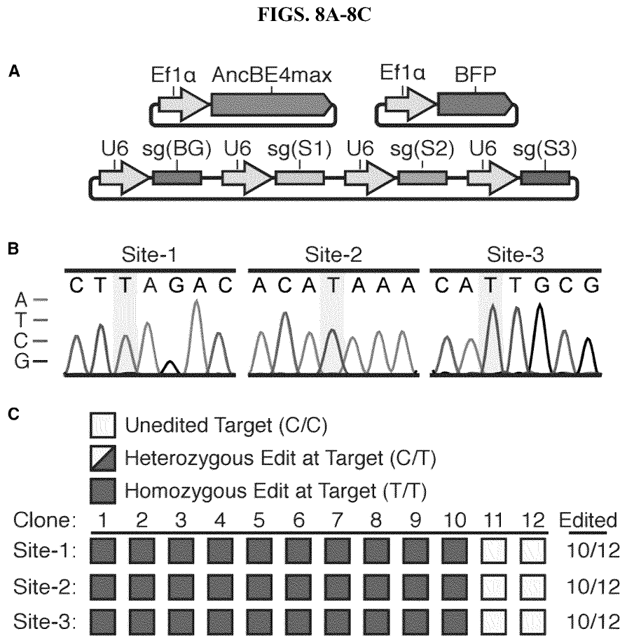

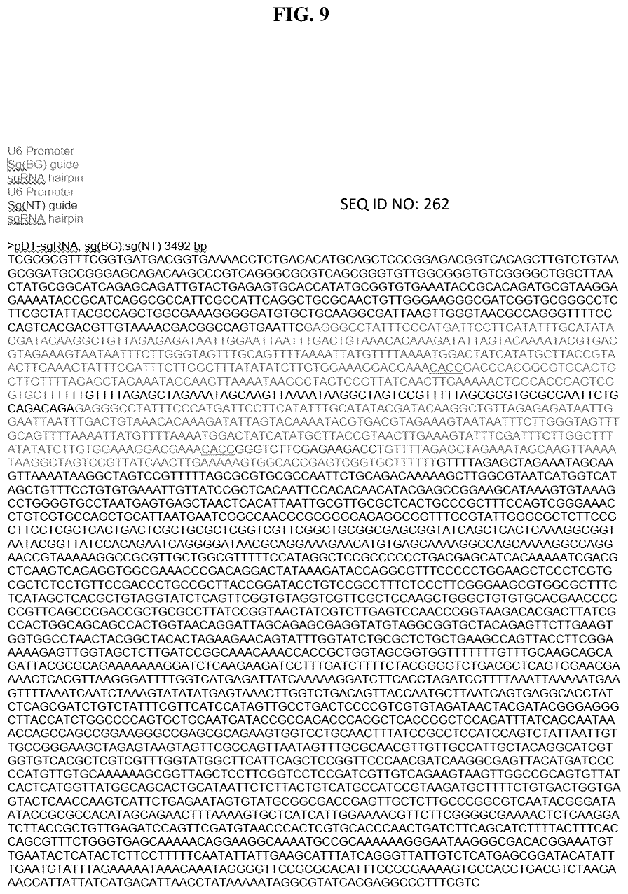

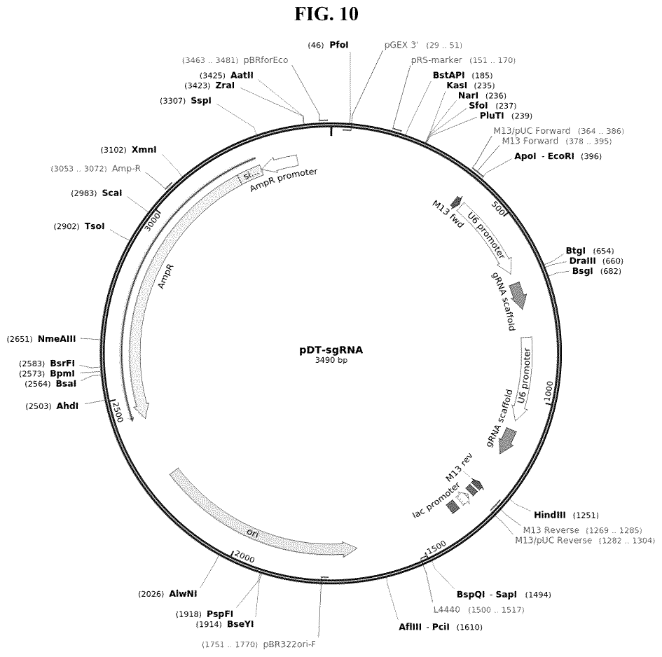

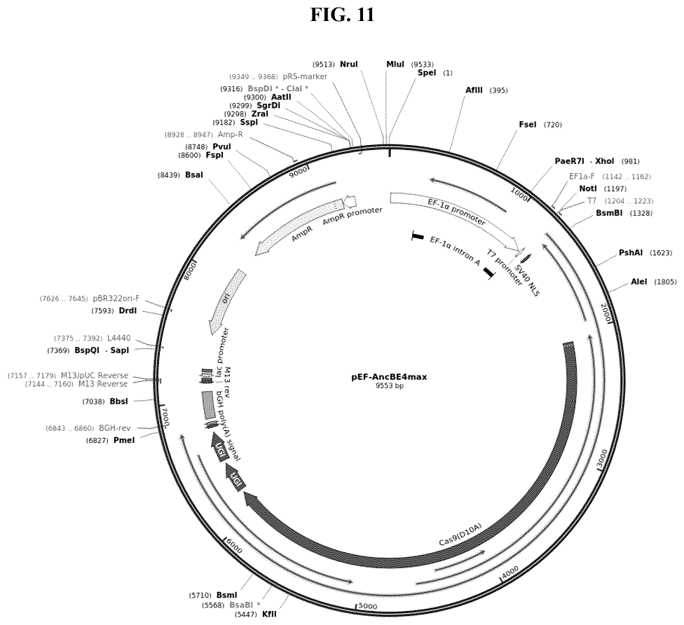

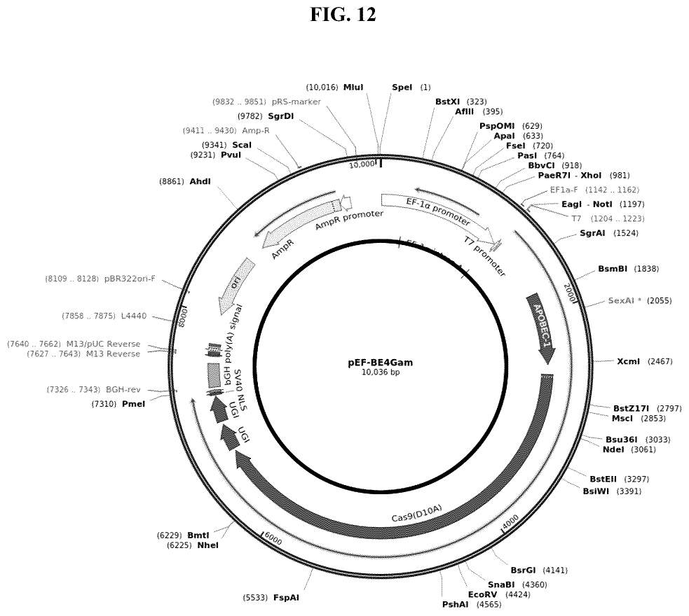

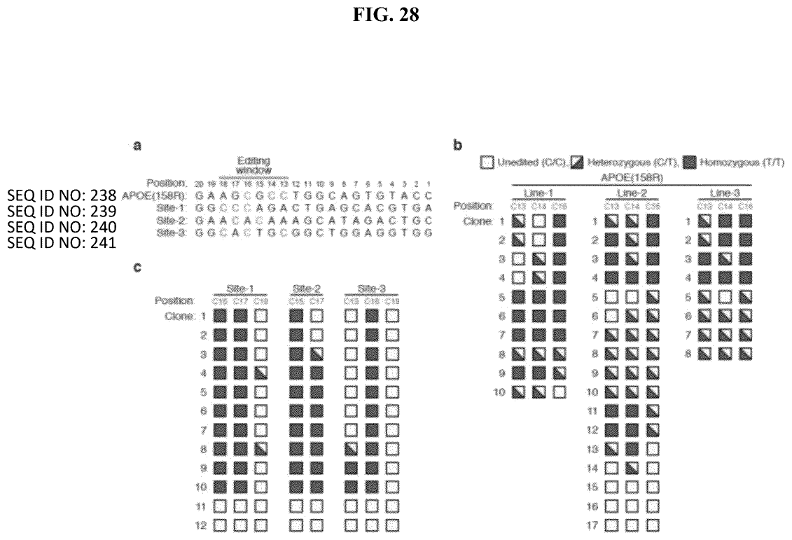

Finally, we wanted to determine if BIG-TREE could be utilized with multiplexed genome modification methods to establish hPSC lines that had been simultaneously edited at multiple genomic locations. Accordingly, a multi-targeting vector (pMT-sgRNA) that contains sg(BG) as well as sgRNAs for three independent genomic target sites (FIG. 8A) was used. Analogous to when BIG-TREE was used to target a single genomic location, TREE was employed to simultaneously target multiple loci by co-transfecting hPSC line 1 with pMT-sgRNA, pEF-BFP, and pEF-AncBE4-max. Sanger sequencing was then performed on the multiplex targeted genomic sites in clonal hPSC lines derived from single GFP-positive cells (FIG. 8B). Along similar lines to when BIG-TREE was used to target a single genomic locus, Sanger sequencing revealed that more than 80% of clones had been targeted at all three sites with all clones displaying biallelic edits (FIG. 8C). Moreover, indels were not identified in any of the clones across all three target sites. Lastly, examination of potential bystander edits within the editing window (FIG. 28A) revealed a number of clones in which at genomic site 2 and site 3 modification only occurred at the target C and not any other Cs within the editing window (FIG. 28C). Specifically, of the ten clones that had homozygous edits at the target C at all three sites, two clones were free from bystander edits at both sites 2 and 3 (clones 1 and 2) and five clones were free from bystander edits at site 3 only (clones 3-7). However, it should be noted that we did not identify any clones in which at genomic site 1 such exclusive modification of the target C occurred. We speculate that because another C occurs immediately adjacent to this target C, that such exclusive modification is likely a rare event that will require site-specific base editors that allow for single-nucleotide changes free from bystander editing at adjacent nucleotides.

### Discussion

In summary, we establish that BIG-TREE is a fast and efficient protocol for the generation of clonal isogenic hPSC lines with homozygous and heterozygous single base pair edits. Because the number of diseases that are a consequence of single point mutations, as well as the growing number of genomic variants of uncertain significance that have been identified through large-scale sequencing efforts, the ability to rapidly engineer isogenic hPSC lines will have a significant impact on the establishment of in vitro models to assess pathogenic risk and dissect disease-causing mechanisms. In addition, in this example, we demonstrate that BIG-TREE can be employed to generate effective loss-of-function cell lines through the introduction of premature stop codons. Currently, most CRISPR/Cas9-based approaches to generate gene knockouts involve the introduction of deleterious DSBs followed by NHEJ-mediated repair that results in frameshift and loss of gene function. As we describe in this example, the ability to rapidly generate gene knockouts without the need for DSBs will have important implications for the use of hPSCs to elucidate the function of specific genes in development and disease. Lastly, we establish that BIG-TREE can allow for the generation of clonal hPSC lines that have been simultaneously edited at multiple independent loci, an important consideration given that many diseases are polygenetic in nature. By comparison, conventional CRISPR/Cas9-based approaches are too inefficient in hPSCs to employ multiplexing editing strategies.

Since the first base editors were engineered, numerous additional base editors with targeting windows, editing efficiencies, PAM specificities, and deaminases have been generated. In the context of BIG-TREE, we employed AncBE4max, which displays a relatively high editing efficiency with low off-target activity. However, one of the limitations of AncBE4max is that it can induce C-to-T conversions at bystander Cs within the editing window. Although bystander editing was a common occurrence in our clonal populations, we did observe clones with exclusive modifications of the target C. More specifically, when generating isogenic lines edited at the APOE(R158) locus, we only isolated one clone that had a monoallelic edit exclusively at the target C. Nonetheless, all of the bystander edits that we observed at the APOE(R158) locus did not impact the amino acid sequence, mitigating the impact on the downstream application of these hPSC lines. With regard to the multiplex editing, we did observe several clones that were free from bystander edits at genomic sites 2 and 3. However, at genomic site 1, where a C is present in the base pair position directly next to the target C, we did not isolate any clones where modification only occurred at the target C. In the future, given the ease of use, we anticipate that utilizing BIG-TREE with these other base editor variants with a narrow editing window will be easily achieved. In this regard, the end-user can select to employ such base editors with a more stringent editing window if editing at a bystander C is not tolerable (e.g., results in changes in the amino acid coding sequence).

In general, there are several enabling aspects to the methods presented in the example that will allow for the facile adoption by a broad set of researchers. First, the high editing frequencies do not require the screening of large numbers of clones to identify those with the desired modification. Moreover, we demonstrate that BIG-TREE is robust, as it allows for the efficient editing of multiple loci and across several independent hPSC lines. Because of these efficiencies, clonal lines can be identified, expanded, and characterized in the course of a few weeks. Along similar lines, the high efficiency of BIG-TREE allows for the biallelic or multiplexed targeting without the need for sequential re-targeting. In addition, BIG-TREE is compatible with off-the-shelf chemical transfection reagents and does not require the cloning of complex viral constructs or the use of specialized cell transfection systems. In fact, all sgRNA vectors were designed to allow for the facile cloning of new target sites via BbsI restriction enzyme digestion and ligation of oligonucleotides that target the desired genomic sequence. Lastly, BIG-TREE offers the flexibility to be used in conjunction with other base editor variants that have altered PAM specificities and editing windows. For instance, the PAM sequence and edit distance can be modified to match the editing specificity and window of the new base editor. Such modifications are straightforward to achieve with the BFP vector using TREE, or the stop codon between RFP and GFP using BIG-TREE. In this manner, BIG-TREE is a readily adoptable method that will enhance and accelerate the use of base-editing approaches in hPSCs.

### Supplemental Experimental Procedures

Human iPSC and HEK293 culture. HPSCs were maintained in mTeSR1 medium (Stemcell Technologies) on feeder-free Matrigel (Corning)-coated plates. Subculture was performed every 3 days using Accutase (Life Technologies) in mTeSR1 medium supplemented with 5 μM Y-27632 (Tocris). Control and AD-patient hPSCs were generated from dermal fibroblasts as previously described (Park et al., 2008).

TABLE 3
HPSC lines described in the example

| Cell Line | Disease Status | MMSE | Mutation |
|---|---|---|---|
| hPSC Line 1 | Non-demented control | 30 | n/a |
| hPSC Line 2 | Familial AD | n/a | APP V717I |
| hPSC Line 3 | Familial AD | n/a | PSEN1 A246E |

mCherry expressing HEK293 line was generated using lentiviral integration of a constitutively expressing mCherry transgene as previously described (Standage-Beier et al., 2019). HEK293 cells were cultured on poly-L-ornithine (4 μg/mL; Sigma Aldrich, St. Louis MO, USA) coated plates in the following media: 1× high glucose DMEM, 10% (v/v) fetal bovine serum, 1% (v/v) L-glutamine penicillin/streptomycin. Culture medium was changed every other day and cells were passaged with Accutase every 5 days.

Plasmid construction. Unless otherwise noted, for all molecular cloning PCRs were performed using Phusion High-Fidelity DNA polymerase (New England Biolabs, Ipswich, MA, USA) using the manufacturer's recommended protocols. All restriction enzyme (New England Biolabs) digests were performed according to the manufacturer's instructions. Ligation reactions were performed with T4 DNA ligase (New England Biolabs) according to the manufacturer's instructions. PCR primers and oligonucleotides were synthesized by Integrated DNA Technologies (Coralville, IA, USA). All PCR products and intermediate plasmid products were confirmed via Sanger sequencing (DNASU Sequencing Core Facility). Complete plasmid sequences will be made available upon request.

For construction of the pEF-BFP plasmid, we utilized PCR to add the H-66 and PAM site mutations into a GFP cassette (Addgene #11154). PCR products containing these mutations were digested with SapI/EcoRI and SapI/NotI and ligated into an EcoRI/NotI digested EF1α expression vector (Addgene #11154).

For construction of the pDT-sgRNA vector, sgRNAs were synthesized as pairs of oligonucleotides (Table 12). Subsequently, 5′ phosphates were added to each oligonucleotide pair by incubating 1 μg oligo nucleotide in 50 μL reactions containing 1×T4 DNA Ligase Buffer (New England Biolabs) and 10 units of T4 Polynucleotide Kinase at 37° C. overnight. Oligonucleotides were then duplexed by heating the kinase reactions to 90° C. on an aluminum heating block for 5 minutes followed by slowly returning the reaction to room temperature over 1 hour. Following duplexing, guides were cloned into a modified pSB1C3 vector containing a U6 promoter, inverted BbsI restriction enzyme digestion sites, and an S. pyogenes sgRNA hairpin. For construction of pMT-sgRNA, pairs of sgRNAs (Table 12) were PCR amplified with primers adding EcoRI/SapI restriction enzyme digestion sites or SapI/XbaI restriction enzyme digestion sites. Purified PCR products were then digested with the respective restriction enzymes and ligated into EcoRI/XbaI digested pUC19 vector (Addgene #50005). The resultant vector contained pairs of sgRNA expression cassettes. To add additional sgRNA expression cassettes, pairs of sgRNAs were PCR amplified with primers that add HindIII/SapI or SapI/HindIII restriction enzyme digestion sites. These products were then digested with HindIII/SapI and ligated into HindIII digested and dephosphorylated pDT-sgRNA vector.

For insertion of the EF1α promoter into pCMV-AncBE4max (Addgene #112094), EF1α was PCR amplified from an EF1α expression vector (Addgene #11154) adding SpeI/NotI restriction enzyme digestion sites. After purification and digestion, these PCR products were ligated into SpeI/NotI digested and dephosphorylated pCMV-AncBE4max vector.

hPSC base editing and clonal isolation. hPSCs were passaged onto Matrigel-coated 12-well plates with 5 μM Y-27632. Media was changed, and transfection were performed 24 hours after passage. 900 ng base editor (pEF1α-AncBE4max), 300 ng sgRNA, and 300 ng pEF1α-BFP was transfected per well using 4 μL Lipofectamine Stem transfection reagent (Life Technologies). Media was changed 24 hours post-transfection. Cells were dissociated using Accutase 48 hours post-transfection and passed through a 0.45 μm filter. Single GFP-positive hPSCs were FACS sorted into 96-well Matrigel coated plates in mTeSR1 supplemented with CloneR (Stemcell Technologies), plates were immediately centrifuged at 100*g for 1 minute and incubated at 37° C. Media was changed 48 hours post-sort with fresh mTeSR1 supplemented with CloneR. 96 hours post-sort, media was changed to mTeSR1 without supplement and clonal hPSC colonies were expanded with fresh media changes daily until ready for subculture.

### Genotyping and Sequence Analysis.

Clones were amplified with the primers listed in Table 13 to determine genotype following base editing. Genomic DNA was prepared from expanded clones using the DNeasy kit (Qiagen) and PCR products were generated with Phusion High-Fidelity Polymerase (New England Biolabs). Amplicons were purified using the QIAquick PCR purification kit (Qiagen) according to manufacturer's instructions prior to Sanger sequencing (Genewiz). For multiplex clones, hPSCs were directly added to a 50 μL master mix consisting of 1× Phire Hot Start II DNA Polymerase (ThermoFisher), 1 μM forward primer, and 1 μM reverse primer. PCR was performed using the following conditions: 98° C. for 5 minutes, followed by 40 cycles at 99° C. for 5 seconds, 56° C. for 5 seconds, and 72° C. for 20 seconds, followed by a final 5 min 72° C. extension. All products sizes were confirmed on a 1% agarose gel prior to Sanger sequencing.

HEK293 transfections. HEK293 cells stably expressing mCherry were transfected in 24 well tissue culture plates at 40% confluence with the following reagents per well: 300 ng pEF1α-AncBE4max, 100 ng sgRNA vector or sg(NT),

0.75 uL Lipofectamine 3000 Transfection Reagent (ThermoFisher), and 1 uL P3000 reagent (Thermo Fisher). Flow cytometry was performed at 7 days post-transfection to evaluate loss of mCherry expression. Genomic DNA was isolated and mCherry was PCR amplified before Sanger sequencing to determine editing efficiency.

Immunofluorescence. Cultures were gently washed twice with PBS prior to fixation. Cultures were then fixed for 15 min at room temperature (RT) with BD Cytofix Fixation Buffer (BD Biosciences). The cultures were then washed twice with PBS and permeabilized with BD Phosflow Perm Buffer III (BD Biosciences) for 30 min at 4OC. Cultures were then washed twice with PBS. Primary antibodies were incubated overnight at 4OC and then washed twice with PBS at room temperature. Secondary antibodies were incubated at RT for 1 hr. Nucleic acids were stained for DNA with Hoechst 33342 (2 μg/mL; Life Technologies) for 10 min at RT and then washed twice with PBS. Antibodies used are as follows at the following concentrations: NANOG (ThermoFisher Scientific; Cat #PA1-097, RRID: AB_2539867; 1:500), OCT4 (ThermoFisher Scientific; Cat #PA5-27438, RRID: AB_2544914; 1:500), SOX2 (ThermoFisher Scientific; Cat #PA1-094, RRID: AB_2539862; 1:500), AFP (Santa Cruz Biotechnology; Cat #sc-15375, RRID: AB_2223935; 1:50), SMA (Santa Cruz Biotechnology; Cat #sc-53015, RRID: AB_628683; 1:50), TUJ1 (Fitzgerald; Cat #10R-T136A, RRID: AB_1289248; 1:1000), Alexa 488 donkey anti-mouse (ThermoFisher Scientific; Cat #A-21206, RRID: AB_2535792; 1:500), and Alexa 488 donkey anti-rabbit (ThermoFisher Scientific; Cat #A-21202, RRID: AB_141607; 1:500).

Tri-lineage differentiation of edited hPSCs. HPSCs were harvested using Accutase and plated on ultra-low attachment plates in mTeSR1 medium. The following day, media was changed to differentiation medium (DM; DMEM/F12, 20% FBS, 1% Pen/Strep). After 5 days, embryoid bodies were plated on Matrigel-coated plates and cultured with DM. After 21 days in DM, cells were fixed, permeabilized, and stained for germ layer markers.

Fluorescence microscopy. All imaging was performed on a Nikon Ti-Eclipse inverted microscope with an LED-based Lumencor SOLA SE Light Engine using a Semrock band pass filter. GFP was visualized with an excitation at 472 nm and emission at 520 nm. BFP was visualized with the DAPI fluorescence channel with excitation at 395 nm and emission at 460 nm. mCherry was visualized with an excitation of 562 nm and emission at 641/75 nm.

Flow cytometry. Cells were dissociated with Accutase for 10 min at 37° C., triturated, and passed through a 40 μm cell strainer. Cells were then washed twice with flow cytometry buffer (BD Biosciences) and resuspended at a maximum concentration of 5×10⁶ cells per 100 μL. Flow cytometry analysis was performed on an Attune NxT (Thermo Fisher Scientific). Flow cytometry files were analyzed using with FlowJo (FlowJo LLC, Ashland, OR, USA).

Off-target analysis. For the data presented in FIG. 28, analysis was performed for the top three off-target loci for sg(BG) and sg(APOE-R158) predicted in silico via CCTop using default parameters for S. pyogenes Cas9 against human genome reference sequence hg38 (Stemmer et al., 2015). Determination of base editing at these off-target sites was performed in a similar manner to that at on-target sites. The PCR primers used to analyze these off-target sites are presented in Table 13.

Quantification of editing in mCherry expressing HEK293 cells. Sanger sequencing of PCRs from genomic DNA from mCherry HEK cells treated with or without base editor and sgRNA were analyzed using EditR (Kluesner et al., 2018). For forward sequencing reactions, the “sgRNA Sequence” was the same as the protospacer. For reverse sequencing reads, the “sgRNA Sequence” was the reverse complement of the protospacer. The 5′ and 3′ start are the corresponding nucleotide number (starting at 1 for the first nucleotide of the sequencing read) 100 bp upstream and downstream of the protospacer, respectively.

Apolipoprotein E (APOE) ELISA. Cells were seeded in a 6 well plate at a density of 3×10⁵ cells per well. Media was changed every 24 hours. On day 3, 24-hour conditioned media was collected and ApoE levels in the medium were measured with the Human APOE(AD2) ELISA Kit (Thermo Scientific).

Statistical analysis. Unless otherwise noted, all data are displayed as mean±standard deviation (S.D).

### SUPPLEMENTAL REFERENCES

Kluesner, M. G., Nedveck, D. A., Lahr, W. S., Garbe, J. R., Abrahante, J. E., Webber, B. R., and Moriarity, B. S. (2018). EditR: A Method to Quantify Base Editing from Sanger Sequencing. CRISPR J. 1, 239-250.
Park, I.-H., Arora, N., Huo, H., Maherali, N., Ahfeldt, T., Shimamura, A., Lensch, M. W., Cowan, C., Hochedlinger, K., and Daley, G. Q. (2008). Disease-specific induced pluripotent stem cells. Cell 134, 877-886.
Stemmer, M., Thumberger, T., Del Sol Keyer, M., Wittbrodt, J., and Mateo, J. L. (2015). CCTop: An Intuitive, Flexible and Reliable CRISPR/Cas9 Target Prediction Tool. PloS One 10, e0124633.

### Example 3-CasMAs (XMAS)-TREE: A Cas9-Mediated Adenosine Transient Reporter for Editing Enrichment

Adenine base editors (ABE) enable single nucleotide modifications without the need for harmful double stranded DNA breaks (DSBs) induced by conventional CRIPSR/Cas9-based approaches. However, most approaches that employ ABEs require inefficient downstream technologies to identify targeted cell populations. This example demonstrates development and characterization of a fluorescence-based method, entitled Cas9-mediated adenosine transient reporter for editing enrichment (CasMAs-TREE; herein abbreviated XMAS-TREE), to facilitate the real-time identification of base-edited cell populations. In particular, this section demonstrates use of XMAS-TREE to detect ABE activity. These studies also demonstrate that, at several independent loci, XMAS-TREE can be used to rapidly identify and purify modified cell populations. In addition, this section demonstrates that XMAS-TREE can be used in concert with multiplex editing schemes to efficiently edit several independent loci. In addition, XMAS-TREE can be used to edit human pluripotent stem cells (hPSCs), a cell type refractory to traditional gene editing approaches. In particular, XMAS-TREE allows for the efficient generation of clonal isogenic hPSCs at loci not editable using typical reporter of transfection (RoT)-based enrichment techniques. Collectively, XMAS-TREE is an easily implemented method that will greatly facilitate the use of ABEs in downstream basic biomedical science and translational applications.

### Results

### Development of a Fluorescent Reporter for Cas9-Mediated Adenosine Base Editing

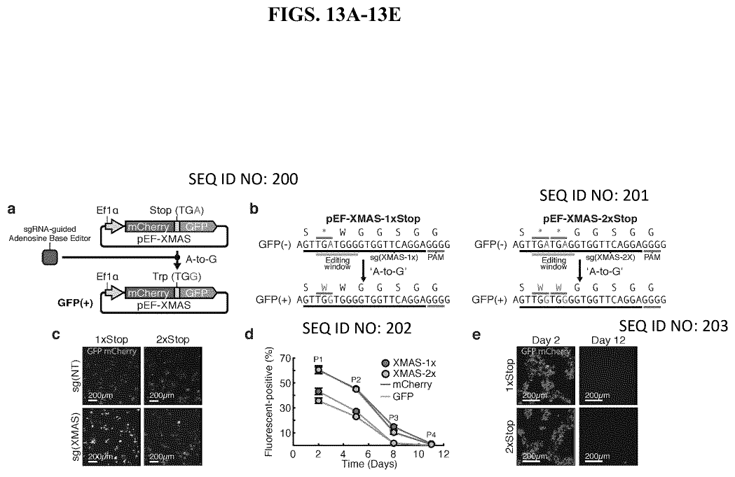

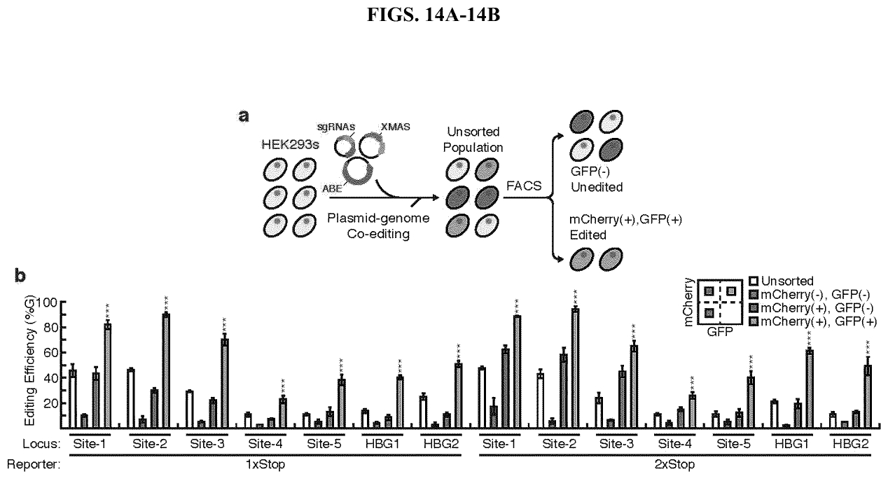

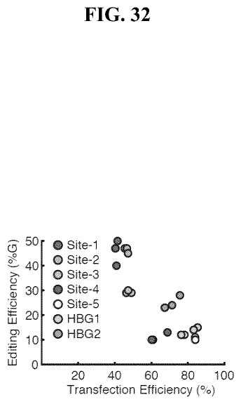

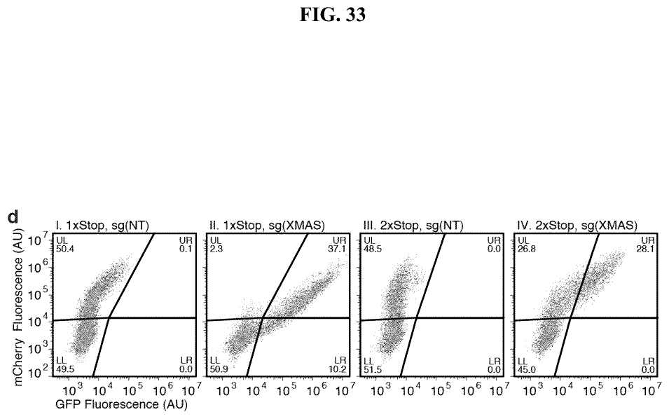

As we have previously shown with cytosine base editors (CBEs), conventional approaches that use reporters of transfection, such as co-transfection or co-expression with a fluorescent protein (herein abbreviated as RoT) only report on the efficiency of plasmid delivery to a cell but not directly on the efficiency of base editing within these cells. To determine if the same was true with adenosine base editing approaches, HEK293 cells were transfected with a reporter plasmid (mCherry), an adenine base editor (ABEmax; pCMV-ABEmax), and a sgRNA for a genomic target site [sg(TS)]. This analysis revealed no correlation between transfection efficiency (percentage of mCherry-positive cells) and editing efficiency (percentage of A-to-G conversion at target nucleotide) (FIG. 32). To that end, we sought to leverage our experience developing fluorescent reporters of editing activity to enable XMAS-TREE. To establish a fluorescent assay to detect ABE activity within a cell, we engineered a construct encoding a mCherry fluorescent protein followed by a stop codon (TGA) immediately preceding the coding sequence for a green fluorescent protein (GFP). Consequently, the A-to-G conversion of that codon to ‘TGG’ (encoding tryptophan) will enable translational read-through and expression of GFP. To determine the utility of this fluorescent-based construct to report on ABE activity, a vector was assembled with a human EF1α promoter to drive expression of the fluorescent reporters (pEF-XMAS; FIG. 13A). In addition, we engineered two versions of this vector, one with a single stop codon (pEF-XMAS-1× Stop) and another with two stop codons (pEF-XMAS-2× Stop; FIG. 13B). It was speculated that A-to-G conversion of two stop codons within the editing window would provide a higher degree of stringency with respect to reporting on base editing activity within a cell. In addition, we designed a sgRNA vector [sg(XMAS)] that would direct the ABE to the target ‘TGA’ resulting in an A-to-G conversion and allow for subsequent translation of the downstream GFP cassette. Next, HEK293 cells were co-transfected with pEF-XMAS, pCMV-ABEmax, and sg(XMAS) or a control non-targeting sgRNA [sg(NT)]. Fluorescence microscopy (FIG. 13C) and flow cytometry (FIG. 13D) revealed that targeting pEF-XMAS with sg(XMAS) resulted in the generation of mCherry/GFP double positive cells, suggesting A-to-G base editing in the target codons allowing for GFP expression. Conversely, targeting pEF-XMAS-1× Stop or pEF-XMAS-2× Stop with sg(NT) did not result in the generation of any GFP positive cells (FIG. 33). Despite similarities in transfection efficiency between pEF-XMAS-1× Stop and pEFXMAS-2× Stop (as measured by percentage of mCherry-positive cells), the percentage of GFP-positive cells was significantly lower in sg(XMAS) targeted cells transfected with pEF-XMAS-2× Stop, suggesting that a higher level of base editing activity was necessary for the activation of GFP expression with the 2× Stop plasmid. Interestingly, a significant percentage of cells that were mCherry-positive were not GFP-positive, verifying that the reporter of transfection (mCherry) does not report on base editing activity within a cell (FIG. 13D). Finally, we wanted to demonstrate that the fluorescent output associated with the XMAS-TREE reporter was transient. As such, the long-term fluorescence of cells transfected with pEF-XMAS and targeted with sg(XMAS) was measured. Indeed, analysis of these cells by flow cytometry (FIG. 13D) and fluorescence microscopy (FIG. 13E) revealed no long-term detectable fluorescent signal, confirming that the XMAS-TREE fluorescent output was transient. Collectively, this data establishes that editing of the XMAS-TREE plasmid provides a transient fluorescent reporter for base editing activity within a cell.

### XMAS-TREE Allows for the Identification and Isolation of Base-Edited Cell Populations

Next, we wanted to demonstrate the utility of XMAS-TREE for the identification and isolation of cells in which targeted genomic adenosine base editing had occurred. To facilitate this, a dual-targeting sgRNA (pDT-sgRNA) vector that contains both sg(XMAS) and a guide matching an endogenous target site, sg(TS) was designed. Additionally, the pDT-sgRNA vector was designed to allow for the straightforward cloning of new target sites via BbsI restriction enzyme digestion and ligation of sg(TS) oligonucleotides. We designed pDT-sgRNA vectors with sequences targeting five genomic loci (Sites 1-5) as well as the promoter of the γ-globin genes HBG1 and HBG2. To utilize XMAS-TREE for enrichment of cells that have been edited at a specific genomic location, we co-transfected these pDT-sgRNA vectors with pEF-XMAS-1× Stop or 2× Stop and pCMV-ABE into HEK293 cells (FIG. 14A). Flow cytometry was then used to isolate reporter positive cell populations and Sanger sequencing was performed on the targeted genomic sites in isolated populations (FIG. 14A). As expected, mCherry-positive/GFP-positive cells were enriched for edited cells when compared to double-negative cell populations (FIG. 14B). Importantly the transfection marker mCherry-positive population had significantly reduced editing compared to the editing reporter positive GFP-positive population. This demonstrates the benefit of utilizing a real-time reporter of base editing. (FIG. 14B). Finally, comparison of mCherry-positive/GFP-positive cells isolated using the 2× Stop versus the 1× Stop vector revealed that use of the 2× Stop vector led to increased editing efficiencies, especially at loci (i.e., HBG1, HBG2) that were more resistant to editing. This suggests that at more difficult to edit loci, the 2× Stop plasmid might provide a higher level of stringency necessary to enrich for edited cell populations. Overall, these results confirm that XMAS-TREE could be used to identify and enrich for adenosine base edited cell populations at a variety of genomic target sites.

### XMAS-TREE Enables Efficient Multiplex Base Editing at Genomic Loci

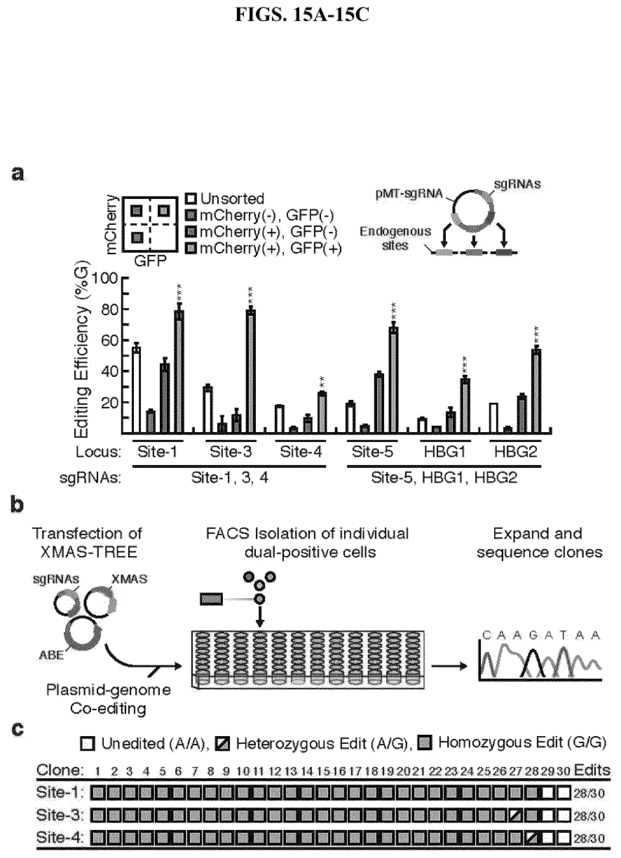

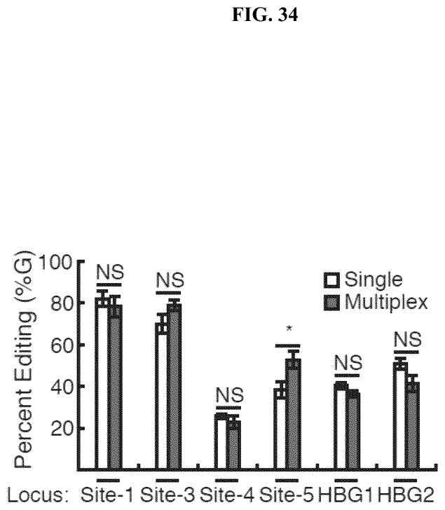

XMAS-TREE was further evaluated to determine if it could be used for multiplexed genome editing. To that end, we generated a multi-targeting vector (pMT-sgRNA) that contains sg(TREE) as well as sgRNAs for multiple genomic targets. More specifically, we generated two pMT-sgRNA vectors—one that would target Site-1/Site-3/Site-4 and another that would simultaneously edit Site-5/HBG1/HBG2. We employed XMAS-TREE to simultaneously target multiple genomic sites by co-transfecting HEK293 cells with pMT-sgRNA, pEF-XMAS, and pCMV-ABEmax. Reporter-positive and -negative cells were isolated by flow cytometry and analyzed by Sanger sequencing at the targeted loci. Consistent with single locus targeting, mCherry-positive/GFP-positive cells displayed a significantly higher frequency of base editing at the target sites than editing levels that were observed in unsorted, mCherry-negative/GFP-negative, and mCherry-positive/GFP-negative cell populations (FIG. 15A). Importantly, there was no significant reduction in editing efficiency when XMAS-TREE was used to target these sites individually or a multiplexed fashion (FIG. 34).

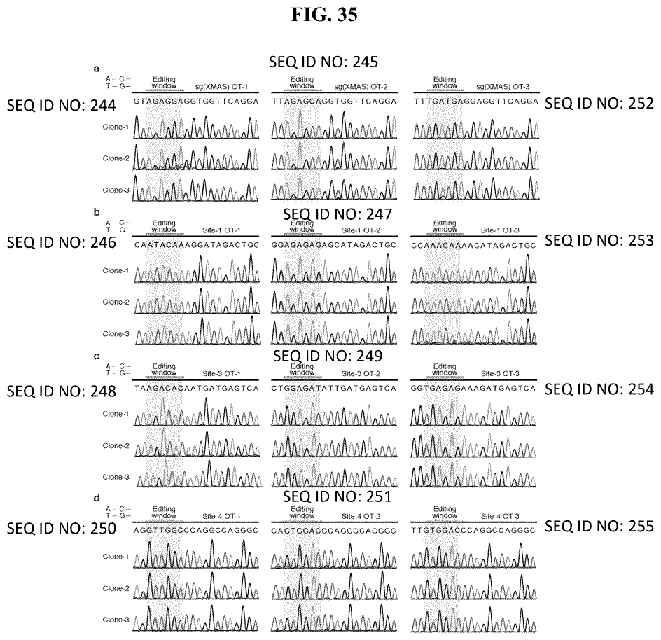

Initial analysis of bulk sorted mCherry-positive/GFP-positive cells suggested that multiplexed editing with XMAS-TREE resulted in a large percentage of cells that had been simultaneously edited at multiple loci. To verify this observation, XMAS-TREE was used for the clonal isolation of base edited populations (FIG. 15B). Briefly, we co-transfected HEK293 cells with pEF-XMAS, pCMV-ABEmax, and a pMT-sgRNA designed to simultaneously target genomic Site-1/Site-3/Site-4. Single GFP-positive cells were sorted into a 96-well plate and expanded prior to analysis. Genomic DNA was isolated from clonal populations and the multiplexed genomic sites were subject to Sanger sequencing after PCR amplification. Remarkably, this analysis revealed that greater than 90% of the clones isolated had been edited, with 26 out of the 30 clones having biallelic conversions at all three genomic loci (FIG. 15C). In addition, we did not observe indels in any of the clones at these target sites. Lastly, we wanted to determine if XMAS-TREE increased A-to-G conversion at off-target loci. Therefore, in several clones that had biallelic edits at all three target sites, we performed off-target analysis at the top predicted sites for sg(XMAS) as well as the sgRNAs used to target Site-1/Site-3/Site-4. At all of the off-target sites analyzed, we did not observe substantial A-to-G edits at these off-target loci (FIG. 35). In addition, indels were not observed at any of the off-target sites in the clones analyzed. Collectively, these results demonstrate the broad utility of XMAS-TREE to allow for the highly efficient, simultaneous editing of multiple independent loci.

Highly Efficient Editing of Human Pluripotent Stem Cells (hPSC's) Using XMAS-TREE

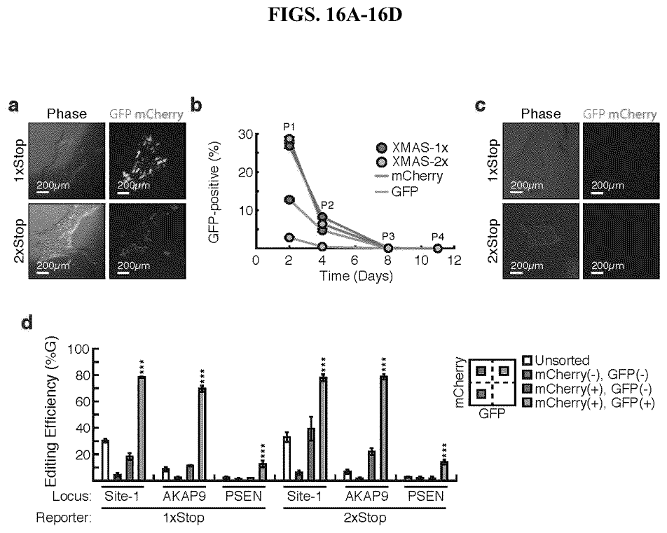

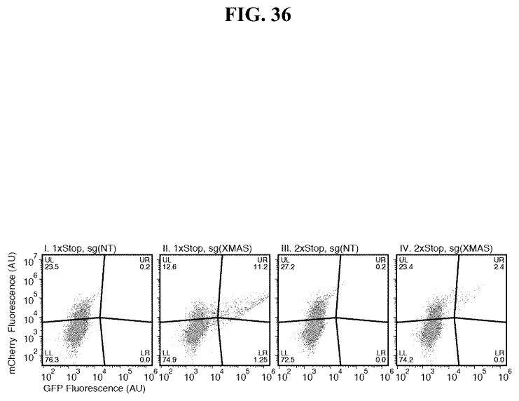

Traditional CRISPR-based approaches to modify single base pairs in hPSCs suffer from extremely low efficiencies. Therefore, we wanted to determine if XMAS-TREE could be utilized to efficiently mediate A-to-G conversions at specific loci in hPSCs. To confirm that the XMAS reporter was functioning in hPSCs, we transfected hPSCs with pEF-XMAS1× Stop/2× Stop, pEF-ABEmax, and sg(XMAS) or sg(NT). Similar to our experiments with HEK293 cells, fluorescence microscopy (FIG. 16A) and flow cytometry (FIG. 16B) with sg(XMAS), but not with sg(NT) (FIG. 36), resulted in the generation of mCherry-positive/GFP-positive cells, indicative of adenosine base editing of the pEF-XMAS reporter plasmid. Additionally, this analysis revealed that the proportion of cells that were positive for the base editing reporter (GFP) relative to the transfection reporter (mCherry) were markedly reduced in hPSCs, consistent with reports that hPSCs are recalcitrant to genomic modification. In this vein, these results suggest that purifying hPSC populations solely with a reporter of transfection (mCherry) would significantly dilute out cells with targeted genomic base edits. In addition, the level of base editing of the 2× Stop plasmid was significantly lower that than observed with the 1× Stop, suggesting that the 2× Stop plasmid provides a higher degree of stringency in identifying base edited populations in hPSCs. Finally, flow cytometry (FIG. 16B) and fluorescence analysis (FIG. 16C) demonstrated that there was no detectable mCherry or GFP signal after 2 weeks of culture, confirming that the fluorescent signal associated with the XMAS-TREE reporter was transient in hPSCs.

Since we established pEF-XMAS reports on functional base editing in hPSCs, we wanted to determine if XMAS-TREE could be employed to enrich for cells with single-base pair edits at target loci in hPSCs. In this regard, we co-transfected hPSCs with pEF-XMAS1× Stop/2× Stop and pEF-ABEmax along with a pDT-sgRNA targeting genomic Site-1 or single base pair changes in AKAP9 and PSEN1 that have been previously associated with increased risks of developing Alzheimer's disease (AD). In turn, flow cytometry was used to purify reporter-positive and -negative cell populations and Sanger sequencing was performed on the targeted genomic locations in isolated populations. This analysis demonstrated that mCherry-positive/GFP-positive cells displayed a statistically significant increase in editing efficiency at the target loci when compared to other populations analyzed (FIG. 16D). In fact, in the more difficult to edit loci, AKAP9 and PSEN1, editing was virtually absent in populations not positive for our base editing reporter (GFP). Furthermore, mCherry-positive/GFP-positive cells isolated using the 2× Stop plasmid allowed for greater level of enrichment. Together, these results demonstrated that XMAS-TREE can been used for the isolation of base-edited hPSC populations.

XMAS-TREE Enables Highly Efficient Generation of Clonal Isogenic hPSC Lines

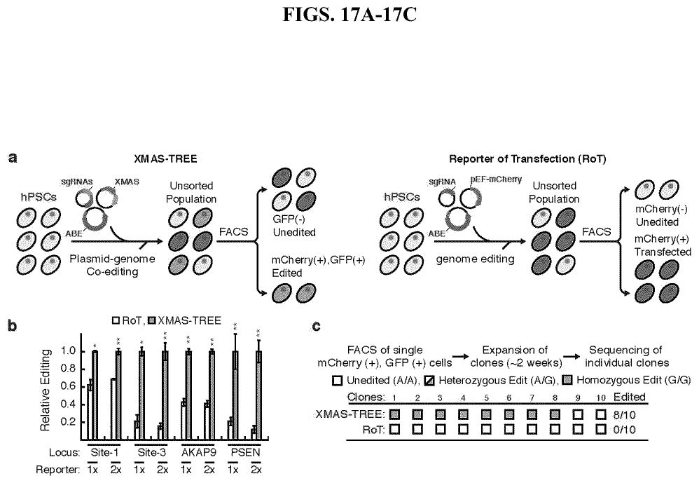

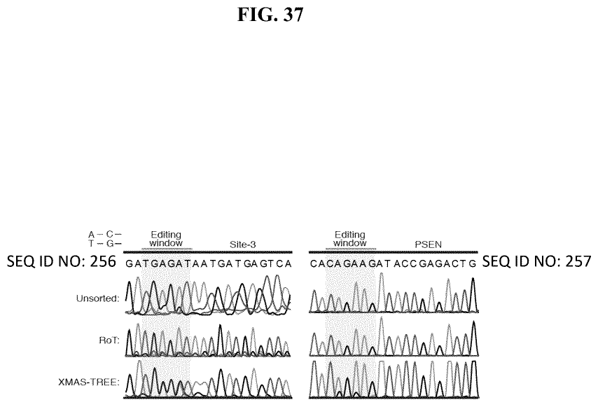

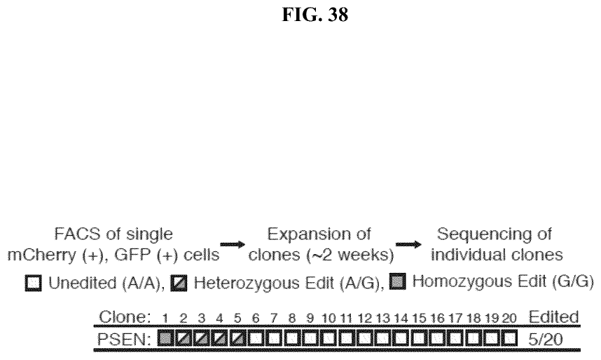

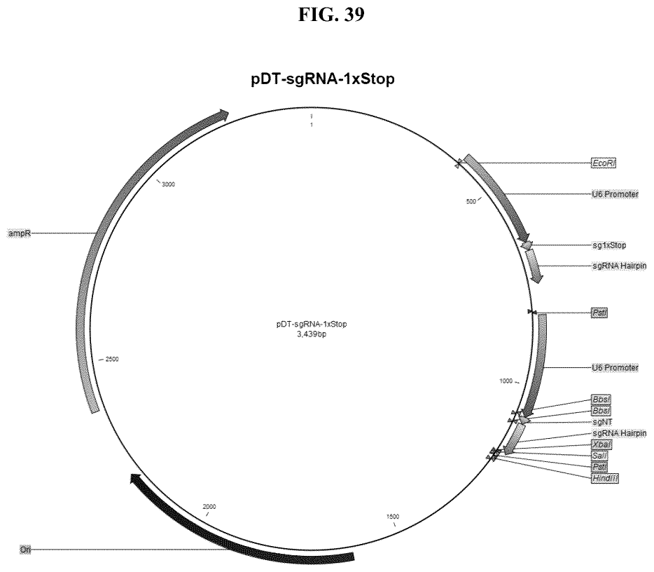

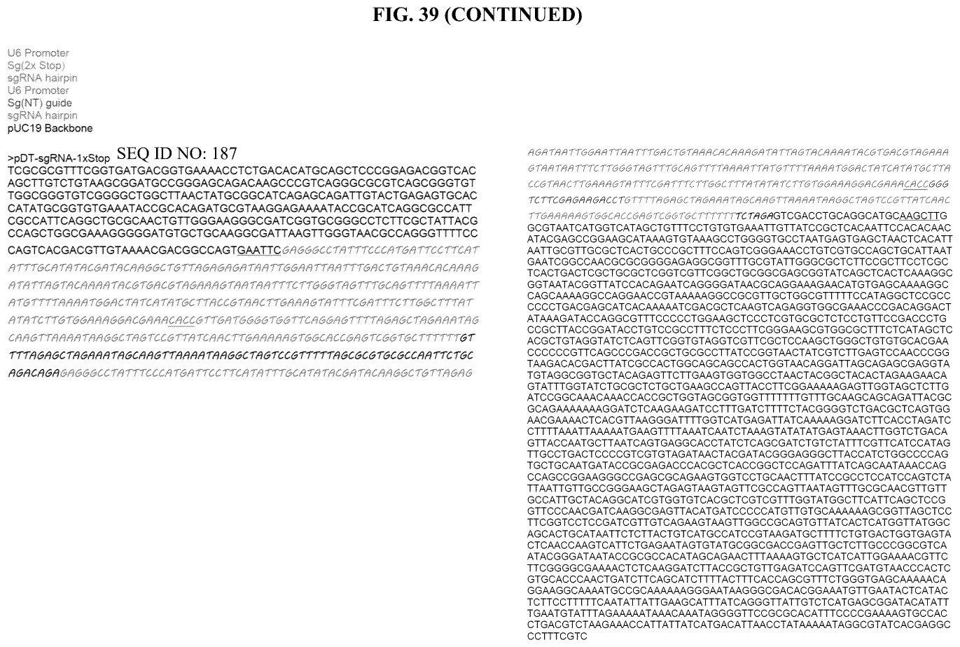

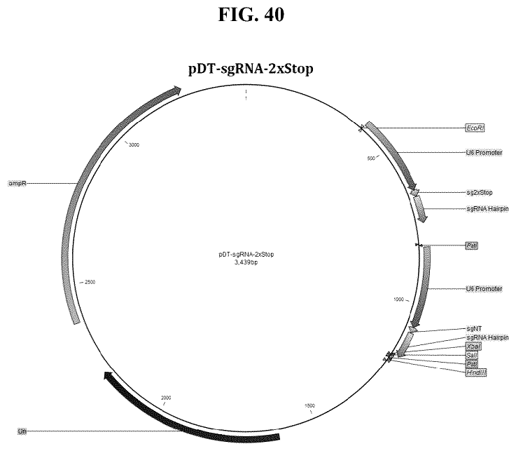

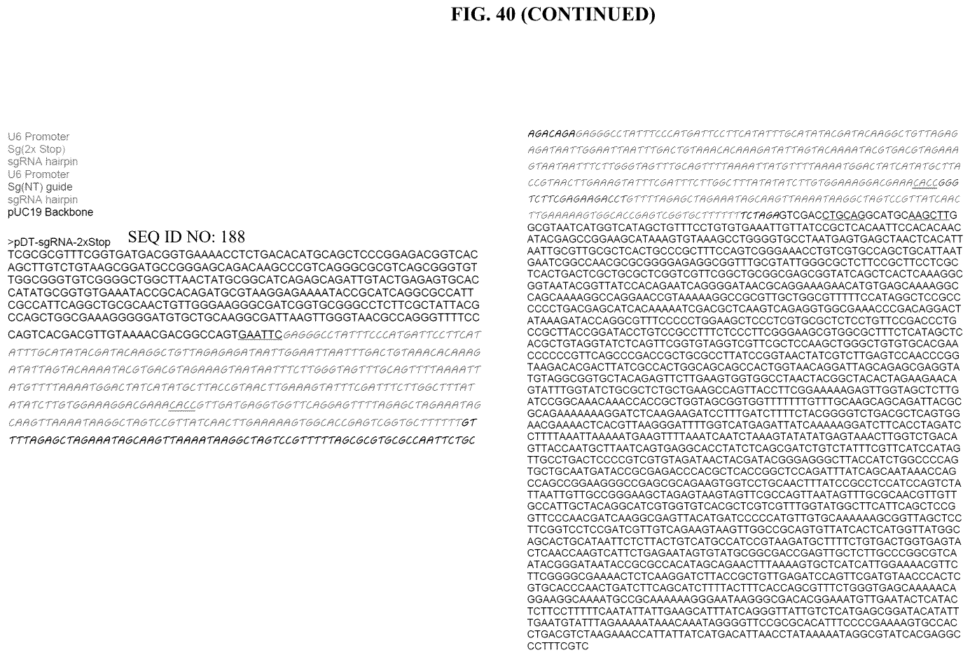

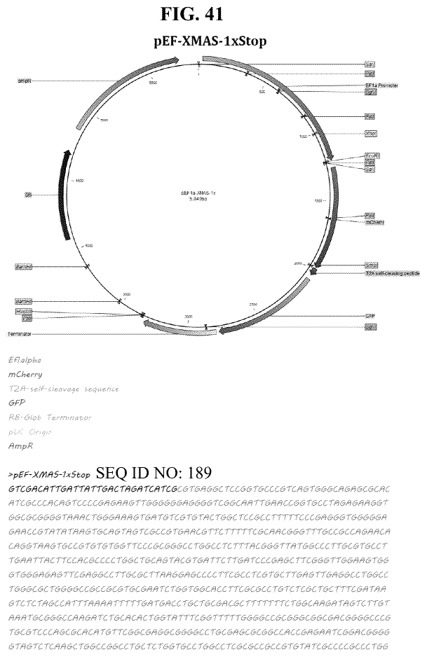

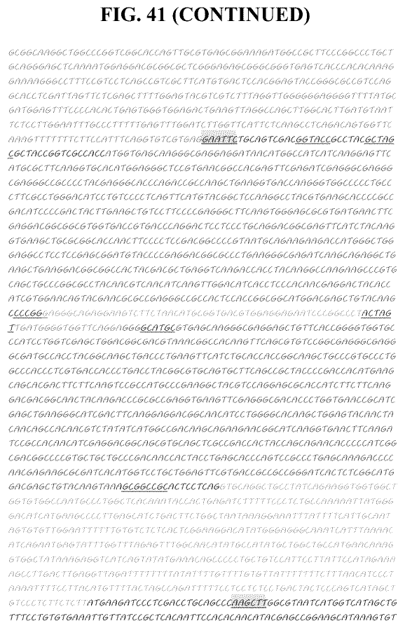

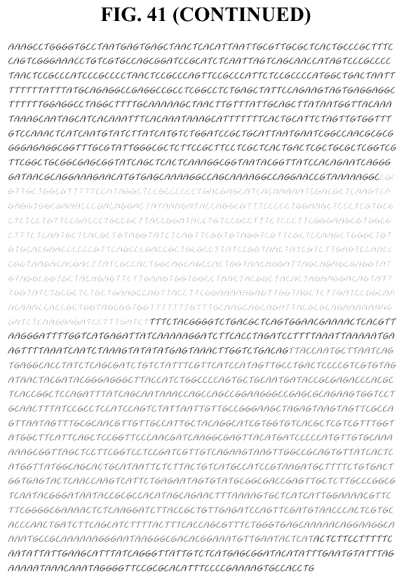

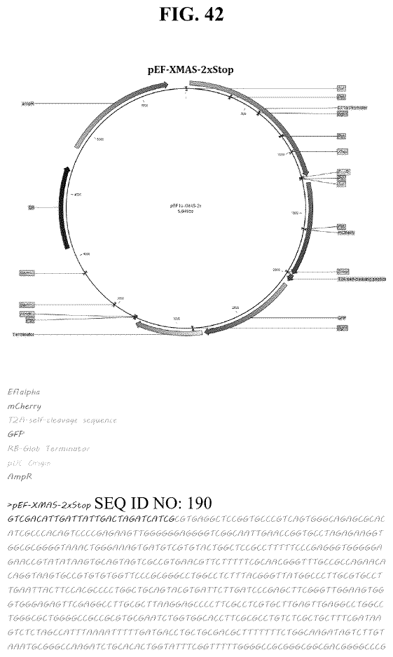

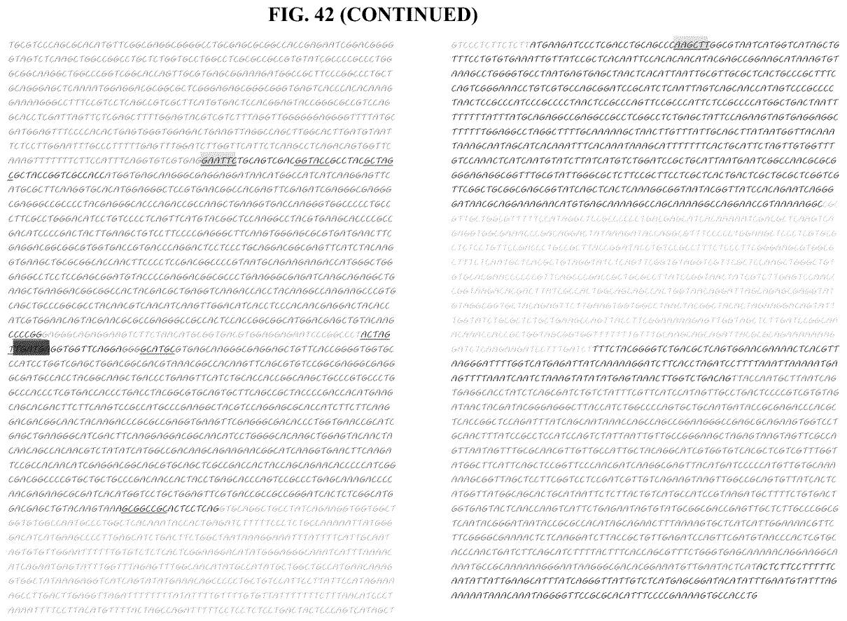

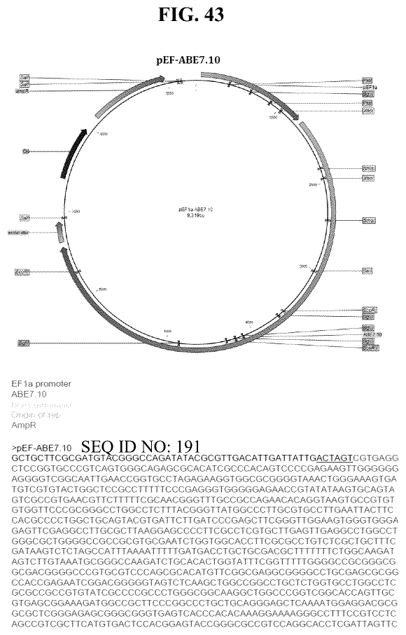

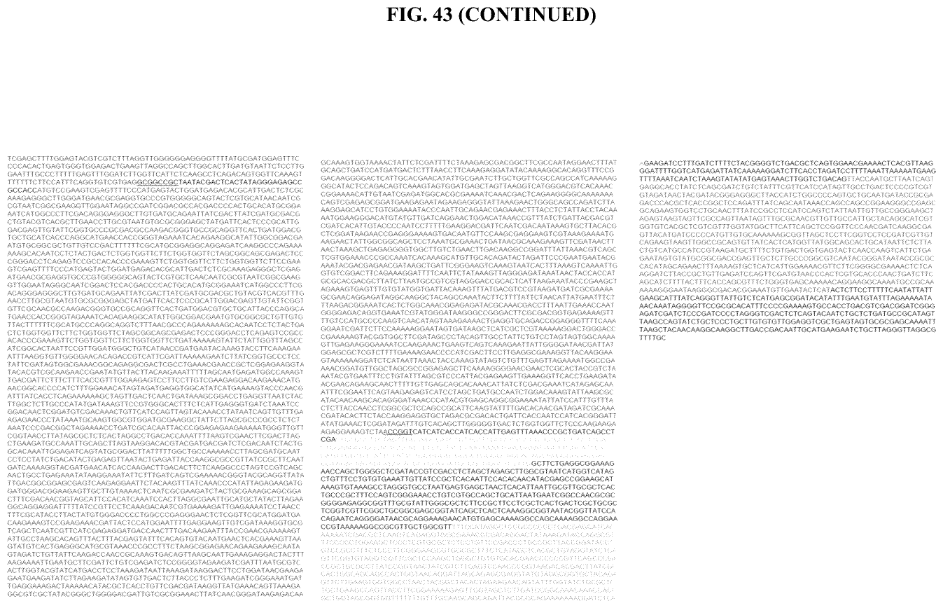

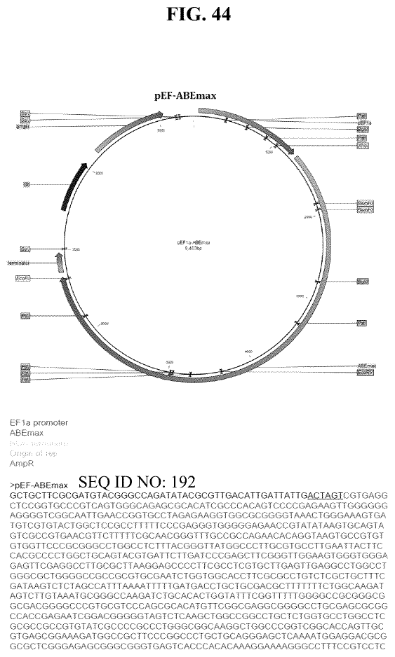

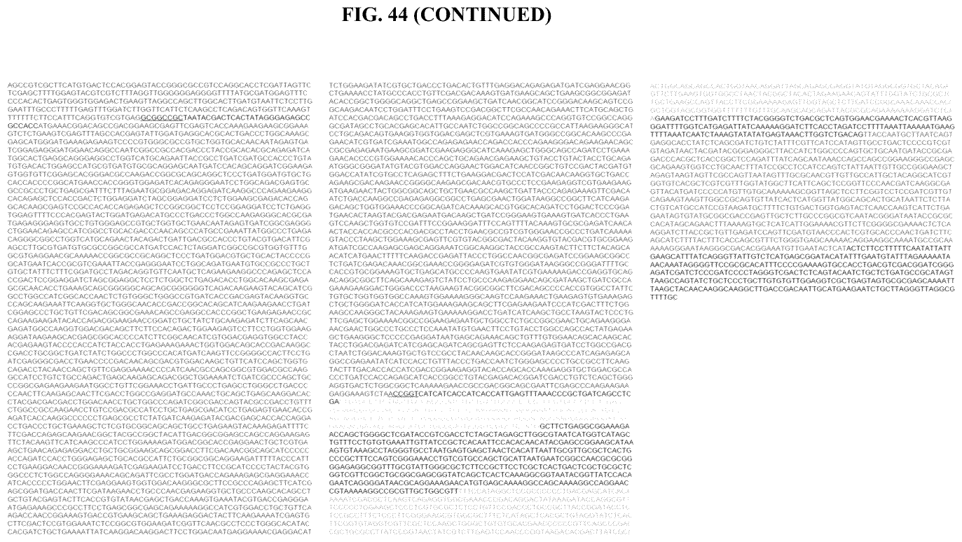

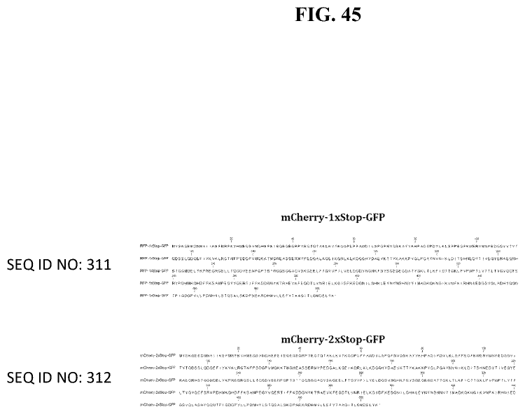

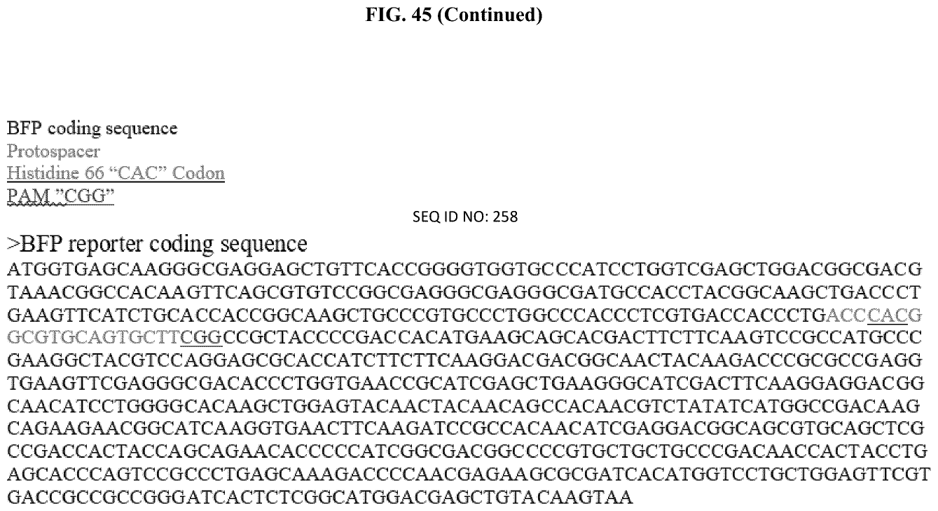

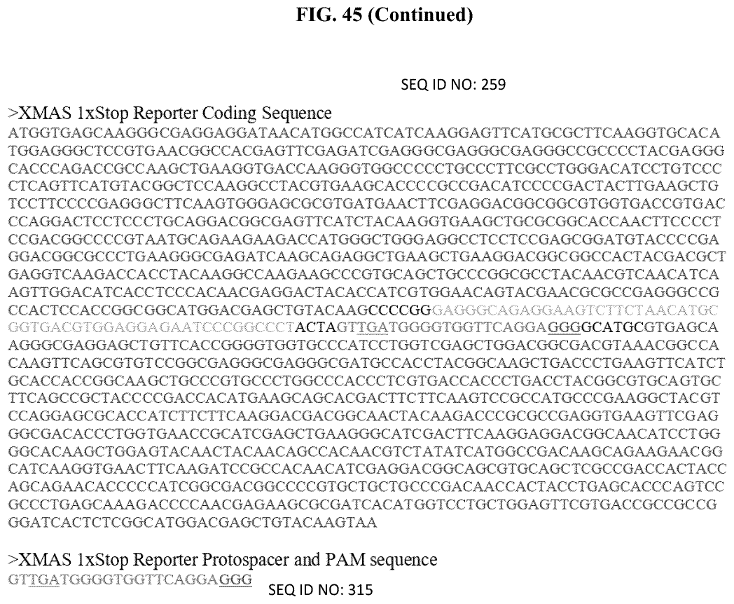

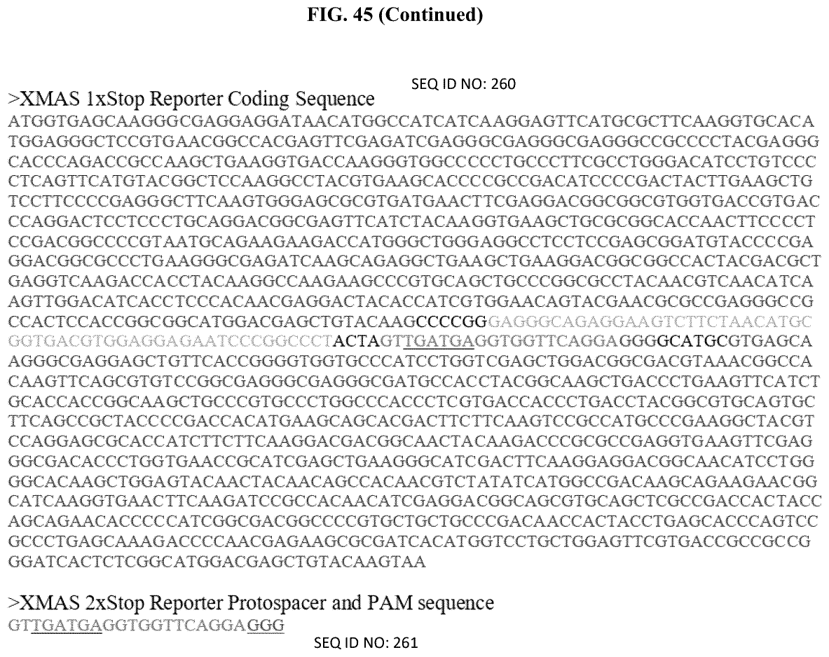

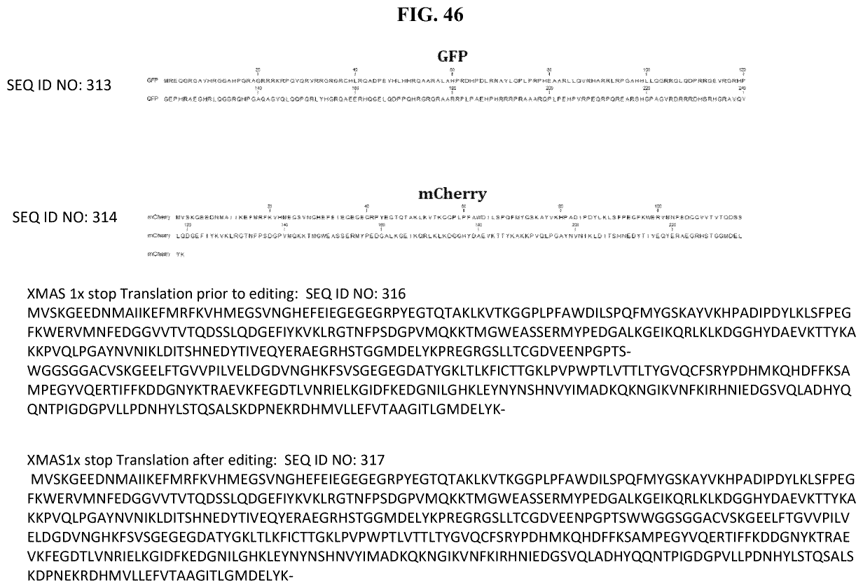

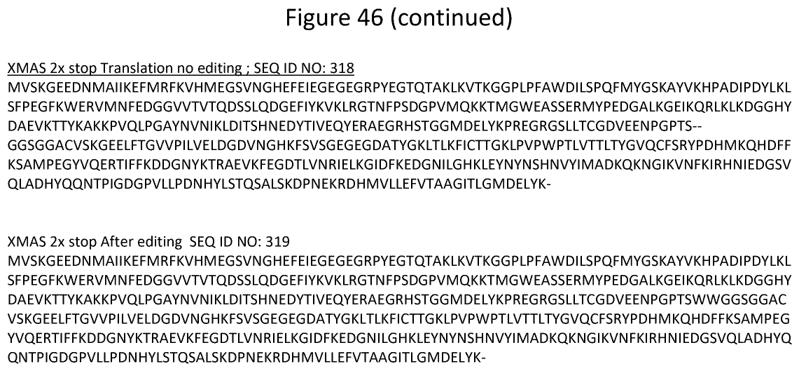

We next wanted to compare the editing efficiency enabled by XMAS-TREE compared to conventional reporters of transfection (RoT). Accordingly, we co-transfected hPSCs with a reporter plasmid (pEF-mCherry), an adenine base editor (pEF-ABEmax), and a sgRNA for various genomic target sites [sg(TS)] (FIG. 17A). Flow cytometry was then used to sort mCherry-positive cell populations (RoT) and Sanger sequencing was performed on the targeted genomic sites. This analysis revealed that across all targeted sites that mCherry-positive/GFP-positive cells isolated using XMAS-TREE had a significantly higher frequency of base editing than mCherry-positive cells isolated using traditional ROT approaches (FIG. 17B). In fact, several targeted loci (i.e. Site-3, PSEN) displayed undetectable levels of editing when traditional ROT approaches were applied (FIG. 37). We then wanted to directly compare the efficiency by which XMAS-TREE and RoT-based methods could be utilized to generate clonal isogenic lines modified at these difficult to edit sites. To this end, we transfected hPSCs with pEF-XMAS, pEF-ABEmax, and pDT-sgRNA containing a sgRNA to target genomic Site-3. Single mCherry-positive/GFP-positive cells were sorted into 96-well plates, expanded, and subject to Sanger sequencing. Of the 10 clones analyzed, 80% had a homozygous A-to-G edit at the genomic Site-3 locus (FIG. 17C). Importantly, indels were not identified in any of the clones at the target site. For comparison to a more conventional ROT approach to generate isogenic lines, this same hPSC line was transfected with a plasmid in which the base-editor (ABEmax) was co-transfected with a pEFmCherry vector as well as the same sgRNA for the Site-3 locus. After 48 hours post-transfection, single GFP-positive cells were sorted into 96-well plates. Clonal lines were then passaged, expanded, and subjected to Sanger sequencing at the targeted locus. Notably, analysis of 10 clonal lines revealed that this ROT-based approach did not result in generation of a single isogenic clone at the target site (FIG. 17C). This ability of XMAS-TREE to generate isogenic clonal lines at sites that did not display significant editing in bulk ROT approaches was also confirmed at the PSEN locus (FIG. 38). In sum, these results demonstrate that XMAS-TREE can not only provide for a higher level of enrichment of base-edited cell populations compared to ROT approaches, but also can allow for the generation of isogenic lines at genomic loci that are not achievable with conventional ROT methods.

Multiplex Editing of hPSC's Using XMAS-TREE

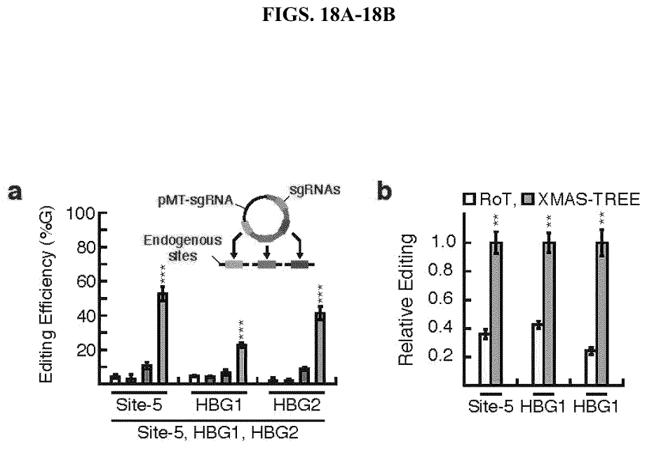

Lastly, we wanted to establish that XMAS-TREE could allow for multiplexed genome modification in hPSCs. HPSCs were co-transfected with pEF-XMAS, pEF-ABEmax, and a pMT-sgRNA with sgRNAs targeting Site-5, HBG1, and HBG2. Similar to our results obtained with HEK293 cells, mCherry-positive/GFP-positive cells had a statistically significant higher level of base editing at all three target sites when compared to those in unsorted, mCherry-negative/GFP-negative, and mCherry-positive/GFP-negative cell populations (FIG. 18A). In addition, direct comparison of multiplex editing using XMAS-TREE and ROT approaches demonstrated that XMAS-TREE allowed for a statistically significant higher level of base editing than by RoT-based methods (FIG. 18B). Altogether, this data demonstrates that XMAS-TREE enables efficient simultaneous editing of multiple loci in hPSCs.

### Discussion

Together, CBEs and ABEs have the potential ability to modify up to 60% of the disease-causing point mutations. That said, BEs can be used in the context of cellular models of human disease models to establish genotype-to-phenotype relationship associated with genetic risk factors, investigate disease mechanisms, and test therapeutic strategies. In our previous work, we describe the development of a transient reporter for editing enrichment (TREE) as a fluorescence-based assay to report on cytosine base editing (CBE) activity within a single cell. In this work, we develop an analogous reporter system, Cas9-mediated adenosine transient reporter for editing enrichment (CasMAs-TREE; XMAS-TREE) that allows for the real-time detection of adenosine base editing for the identification and enrichment of base-edited cell populations. Notably, at several loci, XMAS-TREE allows for the targeted gene editing at efficiencies approaching 90%. As part of these efforts, we also utilized XMAS-TREE to enrich for cells that have been edited at several disease-relevant loci including those associated with sickle-cell anemia (i.e., HBG1, HBG2) and Alzheimer's disease (i.e., AKAP9, PSEN1). In addition, we demonstrate that XMAS-TREE can be used in the context of multiplex genome engineering strategies to facilitate simultaneous A-to-G (or T-to-C) conversions at several independent loci at the same efficiencies when single loci were targeted. Critically, the ability of XMAS-TREE to generate clonal lines that had been simultaneously edited at multiple loci will enable the facile generation of cell-based models of polygenetic diseases. Finally, we establish that the same XMAS-TREE-based methods can be applied in human pluripotent stem cells (hPSCs), a cell population in which gene editing technologies, including base editors and multiplex genome modification, have been challenging to implement. In particular, we show that XMAS-TREE can facilitate the establishment of isogenic hPSC lines at loci that were not able to be modified using well-accepted reporter of transfection (RoT) methods. In fact, we show that at certain target sites that XMAS-TREE can allow for derivation of isogenic clonal populations with biallelic modification with 80% efficiency. Notably, all targeted clones were free from indels at all on-target sites. The clonal targeting efficiencies that we observe with XMAS-TREE in hPSCs are significantly higher than those previously reported with other CRISPR/Cas9-based methods, which are often in the single digits at most loci. In addition, the inefficiencies associated with these well-established methods make it difficult to achieve homozygous or multiplexed editing in hPSCs.

We speculate that XMAS-TREE can be utilized in other applications not described in this example. For example, several groups have reported the generation of additional ABEs with non-NGG PAM specificities, narrower targeting windows, and reduced by-product formation34-36. Accordingly, future application of XMAS-TREE with these next-generation ABE variants will be straightforward. In addition, we anticipate that XMAS-TREE can be applied to induce alterations in target gene expression. More specifically, we previously described how CBEs can be used with other TREE-based strategies to generate gene knockout lines without the introduction of DSBs through in-frame conversion of ‘CAG’ codon encoding for glutamine to a ‘TAG’ premature stop codon. However, these approaches do not allow targeting for all genes and can be limited by the propensity of CBEs to induce genome-wide Cas9-independent off-target mutations. As an alternative, Wang and colleagues recently described an ABE-mediated strategy to induce gene knockout through modification of the ATG start codon to ACG or GTG. Moving forward, XMAS-TREE can be utilized with such strategies to enrich for cell populations with targeted gene knockouts.

In summary, there are several features of XMAS-TREE based methods that will enable extensive use by the research community. First, XMAS-TREE only requires the use of common lipid-based reagents for cell transduction. We envision that XMAS-TREE is compatible with other DNA delivery systems (i.e., electroporation) or expression methods (i.e., ribonucleoprotein complexes [RNP]) that have been utilized in other CRISPR/Cas9- and BE-based genome engineering strategies. In the future, XMAS-TREE associated plasmids can also be easily cloned into nonintegrating viral vectors to facilitate the development of in vivo gene editing methods2. Second, we have designed the sgRNA vectors to allow for the simple restriction enzyme-based cloning of new target sites. In this regard, we show that XMAS-TREE can allow for the highly efficient editing of a diverse set of loci across multiple cell lines. In the future, XMAS-TREE can be easily utilized in other animal, primary, or immortalized cell types. In addition, because of the high editing efficiencies associated with XMAS-TREE, establishment of clonal lines with the targeted base pair edit does not require the screening of hundreds of clones, which is typical of other methods. Finally, we demonstrate that the use of XMAS-1× Stop and -2× Stop plasmids allows the end-user to balance the need for cell yield versus editing stringency. Specifically, the XMAS-1× Stop plasmid provides for a higher degree of cell yield compared to the XMAS-2× Stop plasmid while allowing for enrichment of editing cells at levels higher than conventional approaches. Alternatively, the XMAS-2× Stop plasmid enables a higher degree enrichment at the target loci, especially at difficult to edit genomic locations. Collectively, these enabling features of XMAS-TREE will significantly enhance the use of ABE-based technologies in a variety of contexts and cell populations.

It should be noted that the above description, attached figures and their descriptions are intended to be illustrative and not limiting. Many themes and variations of this disclosure will be suggested to one skilled in this and, in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

TABLE 4
List of sgRNA sequences used in this example.

| Site | Sequence (5′→3′) |
|---|---|
| Site-1 | GGCCCAGACTGAGCACGTG |
| | A |
| | (SEQ ID NO: 14) |
| Site-2 | GAACACAAAGCATAGACTG |
| | C |
| | (SEQ ID NO: 15) |
| Site-3 | GGCACTGCGGCTGGAGGTG |
| | G |
| | (SEQ ID NO: 16) |
| APOE(R158) | GAAGCGCCTGGCAGTGTAC |
| | C |
| | (SEQ ID NO: 17) |
| BFP(H66Y) | GACCCACGGCGTGCAGTGCT |
| | T |
| | (SEQ ID NO: 18) |
| C1ORF228 | GTGCTGTTAGCACCCTGGAA |
| | A |
| | (SEQ ID NO: 19) |

TABLE 5
List of primers used in this example to amplify on-target sites.

| Primer | Forward Sequence (5′→3′) | Reverse Sequence (5′→3′) |
|---|---|---|
| Site-1 | ATGTGGGCTGCCTAGAAAGG (SEQ ID | CCCAGCCAAACTTGTCAACC (SEQ |
| | NO: 20) | ID NO: 21) |
| Site-2 | CCAGCCCCATCTGTCAAACT (SEQ ID | TGAATGGATTCCTTGGAAACAATGA |
| | NO: 22) | (SEQ ID NO: 23) |
| Site-3 | TGGTCTTCTTTCCCCTCCCCTGCCCTCC | GGCCTGGAGGCGGGGGCTCAGAGA |
| | (SEQ ID NO: 24) | (SEQ ID NO: 25) |
| APOE(R158) | GGACGAGACCATGAAGGAGTTGAAGGC | CCACCTGCTCCTTCACCTCGTCCAG |
| | (SEQ ID NO: 26) | (SEQ ID NO: 27) |

TABLE 6
Parameters for EditR analysis.

| Target | Sequencing | | | |
|---|---|---|---|---|
| Site | Direction | Protospacer | 5′ bound | 3′ bound |
| Site 1 | Forward | GGCCCAGACTGAGCACGTGA | GGCCTGGGTCAA (SEQ ID | TTCCTTTCCTCTG (SEQ ID |
| | | (SEQ ID NO: 28) | NO: 29) | NO: 30) |
| | Reverse | TCACGTGCTCAGTCTGGGCC | GAGGAAAGGAAGCCCTGCT | CAGGCCAGGGCTGGA |
| | | (SEQ ID NO: 31) | (SEQ ID NO: 32) | (SEQ ID NO: 33) |
| Site-2 | Forward | GAACACAAAGCATAGACTGC | CCCGCTGGCCCTGT (SEQ ID | TCAGGCTGGCCCGC (SEQ |
| | | (SEQ ID NO: 34) | NO: 35) | ID NO: 36) |
| | Reverse | GCAGTCTATGCTTTGTGTTC | CCAGCCCGCTGGCCCTGTA | AGCTATTCAGGCT (SEQ ID |
| | | (SEQ ID NO: 37) | (SEQ ID NO: 38) | NO: 39) |
| Site 3 | Forward | GTGGCACTGCGGCTGGAGGT | GATGACAGGCAGGGGCA | CAGCACCAGA (SEQ ID NO: |
| | | (SEQ ID NO: 40) | (SEQ ID NO: 41) | 42) |
| | Reverse | ACCTCCAGCCGCAGTGCC | CCGCGGTGCCCCTGCCT | AAGCGGAGACTCTGGTGC |
| | | (SEQ ID NO: 43) | (SEQ ID NO: 44) | (SEQ ID NO: 45) |
| APOE(R158) | Forward | GAAGCGCCTGGCAGTGTACC | CTGCGCAAGCTGCG (SEQ ID | TCGGCGCCCTCGCG (SEQ |
| | | (SEQ ID NO: 46) | NO: 47) | ID NO: 48) |
| | Reverse | GGTACACTGCCAGGCGCTTC | GGATGGCGCTGA (SEQ ID | GCCTCGCCTCCCACC (SEQ |
| | | (SEQ ID NO: 49) | NO: 50) | ID NO: 51) |

TABLE 7
PCR conditions for each target site analyzed by Sanger sequencing.

| | | Denature time and | Annealing time and | Extension time and | Final extension |
|---|---|---|---|---|---|
| | Initial denature time | temperature | temperature | temperature | time and |
| Target | and temperature | 40 cycles | | | temperature |
| Site-1 | 98° C., 45 seconds | 98° C., 10 seconds | 54° C., 5 seconds | 72° C., 20 seconds | 72° C., 10 minutes |
| Site-2 | 98° C., 45 seconds | 98° C., 10 seconds | 56° C., 5 seconds | 72° C., 20 seconds | 72° C., 10 minutes |
| Site-3 | 98° C., 45 seconds | 98° C., 10 seconds | 56° C., 5 seconds | 72° C., 20 seconds | 72° C., 10 minutes |
| APOE(R158) | 98° C., 45 seconds | 98° C., 10 seconds | 62° C., 5 seconds | 72° C., 20 seconds | 72° C., 10 minutes |

TABLE 8
List of primers used in this example to amplify off-target sites. Abbreviations:
BG-OT = Off-targets associated with sg(BG), Site 1-OT = Off-targets associated with
sg(Site-1), Site2-OT = Off-targets associated with sg(Site-2), Site3-OT = Off-targets
associated with Sg(Site-3).

| Primer | Forward Sequence (5′→3′) | Reverse Sequence (5′→3′) |
|---|---|---|
| BG-OT1 | GATGCGCTTCCGGAAGACC (SEQ | GCTTCTTGAGCTTCTCAGCG (SEQ ID NO: |
| | ID NO: 52) | 53) |
| BG-OT2 | GGTAGCATGTTCAGGCACCAG (SEQ | CATCCCTAGTACCGAATCCCATATAGC |
| | ID NO: 54) | (SEQ ID NO: 55) |
| BG-OT3 | CATCCTCCCACCTAAGCCTTTCAA | TTGAGTTAATAGCATTATAACAATTTCCACA |
| | (SEQ ID NO: 56) | (SEQ ID NO: 57) |
| BG-OT4 | ACTCCTTACAACCGGAAGGCAAAC | TGGACGTGGTGAAGCCCGTGGTG (SEQ ID |
| | (SEQ ID NO: 58) | NO: 59) |
| BG-OT5 | TAGGTCTCTAGGGGGCCTCTG (SEQ | AGGCTGCCCAACAGCCCCACT (SEQ ID |
| | ID NO: 60) | NO: 61) |
| Site1-OT1 | TCCCCTGTTGACCTGGAGAA (SEQ | CACTGTACTTGCCCTGACCA (SEQ ID NO: |
| | ID NO: 62) | 63) |
| Site1-OT2 | TGAGATGTGGGCAGAAGGG (SEQ | TTGGTGTTGACAGGGAGCAA (SEQ ID NO: |
| | ID NO: 64) | 65) |
| Site1-OT3 | GTCCAAAGGCCCAAGAACCT (SEQ | TGAGAGGGAACAGAAGGGCT (SEQ ID |
| | ID NO: 66) | NO: 67) |
| Site1-OT4 | GCTCATCTTAATCTGCTCAGCC | TCCTAGCACTTTGGAAGGTCG (SEQ ID |
| | (SEQ ID NO: 68) | NO: 69) |
| Site1-OT5 | AAAGGAGCAGCTCTTCCTGG (SEQ | GTCTGCACCATCTCCCACAA (SEQ ID NO: |
| | ID NO: 70) | 71) |
| Site2-OT1 | GTGTGGAGAGTGAGTAAGCCA | ACGGTAGGATGATTTCAGGCA (SEQ ID |
| | (SEQ ID NO: 72) | NO: 73) |
| Site2-OT2 | TTTTTTGGTACTCGAGTGTTATTCAG | CACAAAGCAGTGTAGCTCAGG (SEQ ID |
| | (SEQ ID NO: 74) | NO: 75) |
| Site3-OT1 | GGCATGGCTTCTGAGACTCA (SEQ | CCCCTTGCACTCCCTGTCTTT (SEQ ID NO: |
| | ID NO: 76) | 77) |
| Site3-OT2 | GAAGAGGCTGCCCATGAGAG (SEQ | TTTGGCAATGGAGGCATTGG (SEQ ID NO: |
| | ID NO: 78) | 79) |
| Site3-OT3 | GGTCTGAGGCTCGAATCCTG (SEQ | CTGTGGCCTCCATATCCCTG (SEQ ID NO: |
| | ID NO: 80) | 81) |
| Site3-OT4 | TTTCCACCAGAACTCAGCCC (SEQ | CCTCGGTTCCTCCACAACAC (SEQ ID NO: |
| | ID NO: 82) | 83) |
| Site3-OT5 | GCAGGGGAGGGATAAAGCAG (SEQ | CACGGGAAGGACAGGAGAAG (SEQ ID |
| | ID NO: 84) | NO: 85) |

TABLE 9
List of primers used in this example for NGS analysis.

| Primer | Forward Sequence (5′→3′) | Reverse Sequence (5′→3′) |
|---|---|---|
| Site-1 | ATGTGGGCTGCCTAGAAAGG (SEQ ID | CCCAGCCAAACTTGTCAACC (SEQ |
| | NO: 86) | ID NO: 87) |
| APOE(R158) | GGACGAGACCATGAAGGAGTTGAAGGC | CCACCTGCTCCTTCACCTCGTCCAG |
| | (SEQ ID NO: 88) | (SEQ ID NO: 89) |

TABLE 10
PCR conditions for each target site subjected to NGS analysis.

| | | Denature time and | Annealing time and | Extension time and | Final extension |
|---|---|---|---|---|---|
| | Initial denature time | temperature | temperature | temperature | time and |
| Target | and temperature | 40 cycles | | | temperature |
| Site-1 | 98° C., 45 seconds | 98° C., 10 seconds, | 54° C., 5 seconds | 72° C., 20 seconds | 72° C., 10 minutes |
| APOE(R158) | 98° C., 45 seconds | 98° C., 10 seconds, | 62° C., 5 seconds | 72° C., 20 seconds | 72° C., 10 minutes |

TABLE 11
Comparison of editing efficiency using RoT-based approaches at the
same target loci in this example, Komar et al., and Koblan et al.

| | | | | FIG. 5C Komar et. al Sci Adv. | | | FIG. 1C Koblan et. al Nat | | |
|---|---|---|---|---|---|---|---|---|---|
| | FIG. 3E Standage-Beier et al. | | | 2017 Aug 30; 3(8) | | | Biotechnol. 2018 October; 36(9): 843-846 | | |
| | Reporter of Transfection | | | No Reporter | | | Reporter of Transfection | | |
| | Unsorted | Reporter− | Reporter+ | Unsorted | Reporter− | Reporter+ | Unsorted | Reporter− | Reporter+ |
| Site-1 | 21.3 ± 2.9 | 3.3 ± 2.8 | 40.7 ± 7.0 | ^{~}45 | N/A | N/A | ^{~}38 | N/A | ^{~}55 |
| (HEK 3) | | | | | | | | | |
| Site-2 | 36.6 ± 3.8 | 13.3 ± 5.9 | 49.7 ± 5.1 | ^{~}35 | N/A | N/A | ^{~}20 | N/A | ^{~}38 |
| (HEK 2) | | | | | | | | | |
| Site-3 | 24.0 ± 6.6 | 7.6 ± 5.0 | 45.3 ± 1.5 | ^{~}45 | N/A | N/A | ^{~}25 | N/A | ^{~}40 |
| (HEK 4) | | | | | | | | | |

TABLE 12
List of sgRNA sequences used in this example.

| Site | Sequence (5′→3′) |
|---|---|
| BFP(H66Y) | GACCCACGGCGTGCAGTGCT |
| | T (SEQ ID NO: 90) |
| Site-1 | GGCCCAGACTGAGCACGTGA |
| | (SEQ ID NO: 91) |
| Site-2 | GAACACAAAGCATAGACTGC |
| | (SEQ ID NO: 92) |
| Site-3 | GGCACTGCGGCTGGAGGTGG |
| | (SEQ ID NO: 93) |
| APOE(R158C) | GAAGCGCCTGGCAGTGTACC |
| | (SEQ ID NO: 94) |
| APOE(Q39X) | GTGGCAGAGCGGCCAGCGCT |
| | (SEQ ID NO: 95) |
| mCh1 | GCACCCAGACCGCCAAGCTG |
| | A (SEQ ID NO: 96) |
| mCh2 | GACCCAGGACTCCTCCCTGC |
| | (SEQ ID NO: 97) |
| mCh3 | GCAAGCAGAGGCTGAAGCTG |
| | A (SEQ ID NO: 98) |
| Non-target (NT) | GGGTCTTCGAGAAGACCT |
| | (SEQ ID NO: 99) |

TABLE 13
List of primer sequences used in this example for on- and off-target sites.
Abbreviations: BG-OT = Off-targets associated with sg(BG), APOE(R158C) = Off-targets
associated with sg(APOE^{R158C})

| Primer | Forward Sequence (5′→3′) | Reverse Sequence (5′→3′) |
|---|---|---|
| Site-1 | ATGTGGGCTGCCTAGAAAGG (SEQ ID | CCCAGCCAAACTTGTCAACC |
| | NO: 100) | (SEQ ID NO: 101) |
| Site-2 | CCAGCCCCATCTGTCAAACT (SEQ ID | TGAATGGATTCCTTGGAAACAATGA |
| | NO: 102) | (SEQ ID NO: 103) |
| Site-3 | TGGTCTTCTTTCCCCTCCCCTGCCCTC | GGCCTGGAGGCGGGGGCTCAGAGA |
| | C (SEQ ID NO: 104) | (SEQ ID NO: 105) |
| APOE(R158C) | GGACGAGACCATGAAGGAGTTGAAGG | CCACCTGCTCCTTCACCTCGTCCAG |
| | C (SEQ ID NO: 106) | (SEQ ID NO: 107) |
| APOE(Q39X) | TCAGAAGGACCCTGACCCACCT (SEQ | ATGAAACCTGGACCTGGGGAGGTATA |
| | ID NO: 108) | (SEQ ID NO: 109) |
| mCherry | AGCTGTGACCGGCGCCTACG (SEQ ID | GGGATTCTCCTCCACGTCAC |
| | NO: 110) | (SEQ ID NO: 111) |
| BG-OT1 | GATGCGCTTCCGGAAGACC (SEQ ID | GCTTCTTGAGCTTCTCAGCG |
| | NO: 112) | (SEQ ID NO: 113) |
| BG-OT2 | GGTAGCATGTTCAGGCACCAG (SEQ ID | CATCCCTAGTACCGAATCCCATATAGC |
| | NO: 114) | (SEQ ID NO: 115) |
| BG-OT3 | CATCCTCCCACCTAAGCCTTTCAA | TTGAGTTAATAGCATTATAACAATTTC |
| | (SEQ ID NO: 116) | CACA (SEQ ID NO: |
| | | 117) |
| APOE(R158C)- | GATACACCATAAAGGGGTTTGACTG | ACCATTTCCCCCCAATTCTACTC |
| OT1 | (SEQ ID NO: 118) | (SEQ ID NO: 119) |
| APOE(R158C)- | CATCTGCATTGGCTTGAAACATC (SEQ | TTACAAAAGTGCTAAATGATGCACAT |
| OT2 | ID NO: 120) | (SEQ ID NO: 121) |
| APOE(R158C)- | ACTCAGTAAAGCTCCTCTTCAAC (SEQ | TTTTGCTTAGGTCCACTGGGC |
| OT3 | ID NO: 122) | (SEQ ID NO: 123) |

TABLE 14
List of sgRNA sequences used in this example.

| | |
|---|---|
| Site | Sequence (5′→3′) |

| | |
|---|---|
| XMAS-1xStop | GTTGATGGGGTGGTTCAGGA |
| | (SEQ ID NO: 124) |
| XMAS-2xStop | GTTGATGAGGTGGTTCAGGA |
| | (SEQ ID NO: 125) |
| Site-1 | GAACACAAAGCATAGACTGC |
| | (SEQ ID NO: 126) |
| Site-2 | GAGTATGAGGCATAGACTGC |
| | (SEQ ID NO: 127) |
| Site-3 | GATGAGATAATGATGAGTCA |
| | (SEQ ID NO: 128) |
| Site-4 | GGATTGACCCAGGCCAGGGC |
| | (SEQ ID NO: 129) |
| Site-5 | GTAGAAAAAGTATAGACTGC |
| | (SEQ ID NO: 130) |
| HBG1 | GCTTGACCAATAGCCTTGACA |
| | (SEQ ID NO: 131) |
| HBG2 | GATATTTGCATTGAGATAGTG |
| | (SEQ ID NO: 132) |
| AKAP9 | GAAAATAGTTGAAGAAAAAG |
| | (SEQ ID NO: 133) |
| PSEN | GCACAGAAGATACCGAGACTG |
| | (SEQ ID NO: 134) |
| Non-target (NT) | GGGTCTTCGAGAAGACCT |
| | (SEQ ID NO: 135) |

TABLE 15
List of primer sequences used in this example.

| Primer | Forward Sequence (5′→3′) | Reverse Sequence (5′→3′) |
|---|---|---|
| Site-1 | TCCTTGGAAACAATGATAACAAGAC | CCAGCCCCATCTGTCAAACT (SEQ |
| | (SEQ ID NO: 136) | ID NO: 137) |
| Site-2 | GCTTATATTCTAGGGAGACAGACAT | ACCTGAGGTCAGAAGTTTGAGA |
| | (SEQ ID NO: 138) | (SEQ ID NO: 139) |
| Site-3 | GTCTGAGGTCACACAGTGGG (SEQ ID | AGAGCAGGGACCACATCTAC (SEQ |
| | NO: 140) | ID NO: 141) |
| Site-4 | GCCAAACTTGTCAACCAGTA (SEQ ID | ATGTGGGCTGCCTAGAAAGG (SEQ |
| | NO: 142) | ID NO: 143) |
| Site-5 | TCCATTTATATGAAATGTTCAGAAAAG | GTAACTATATGCTCTCTGATTCTCC |
| | GCAAAT (SEQ ID NO: 144) | TATTAGC (SEQ ID NO: 145) |
| HBG1 | CCTACCTTCCCAGGGTTT (SEQ ID NO: | AAGAAGTCCTGGTATCTTCTATG |
| | 146) | (SEQ ID NO: 147) |
| HBG2 | TCAGACGTTCCAGAAGCGAG (SEQ ID | GACAAGAAGGTGAAAAACGGCTG |
| | NO: 148) | (SEQ ID NO: 149) |
| AKAP9 | GATTCAAAGCATACCAGAGAATAGT | TCAAACTAGTATGCATTTCAACAAC |
| | (SEQ ID NO: 150) | (SEQ ID NO: 151) |
| PSEN1 | GAGTGTAGCTGTTTTTCTCAGGTT | GAATACCCAACCATAAGAAGAACAG |
| | (SEQ ID NO: 152) | (SEQ ID NO: 153) |

TABLE 16
Phire PCR conditions for each target site analyzed by Sanger sequencing.

| | Initial denature | Denature time | Annealing time | | Extension time | Final extension |
|---|---|---|---|---|---|---|
| | time and | and temperature | and temperature | | and temperature | time and |
| Target | temperature | 40 cycles | | | | temperature |
| Site-1 | 98 C. 5 min | 98 C. 5 sec | 58 C. | 5 sec | 72 C. 30 sec | 72 C. 5 min |
| Site-2 | 98 C. 5 min | 98 C. 5 sec | 62 C. | 5 sec | 72 C. 30 sec | 72 C. 5 min |
| Site-3 | 98 C. 5 min | 98 C. 5 sec | 56.8 C. | 5 sec | 72 C. 30 sec | 72 C. 5 min |
| Site-4 | 98 C. 5 min | 98 C. 5 sec | 61.3 C. | 5 sec | 72 C. 30 sec | 72 C. 5 min |
| Site-5 | 98 C. 5 min | 98 C. 5 sec | 65 C. | 5 sec | 72 C. 30 sec | 72 C. 5 min |
| HBG1 | 98 C. 5 min | 98 C. 5 sec | 59.2 C. | 5 sec | 72 C. 30 sec | 72 C. 5 min |
| HBG2 | 98 C. 5 min | 98 C. 5 sec | 59 C. | 5 sec | 72 C. 30 sec | 72 C. 5 min |
| AKAP9 | 98 C. 5 min | 98 C. 5 sec | 64 C. | 5 sec | 72 C. 30 sec | 72 C. 5 min |
| PSEN1 | 98 C. 5 min | 98 C. 5 sec | 63 C. | 5 sec | 72 C. 30 sec | 72 C. 5 min |

TABLE 17
Parameters for EditR analysis.

| Target | | | |
|---|---|---|---|
| Site | Protospacer | 5′ bound | 3′ bound |
| Site-1 | GAACACAAAGCATAGAC | 50 | 120 |
| | TGC (SEQ ID NO: 154) | | |
| Site-2 | GAGTATGAGGCATAGAC | 140 | 200 |
| | TGC (SEQ ID NO: 155) | | |
| Site-3 | GATGAGATAATGATGAG | 100 | 180 |
| | TCA (SEQ ID NO: 156) | | |
| Site-4 | GGATTGACCCAGGCCA | 80 | 160 |
| | GGGC (SEQ ID NO: 157) | | |
| Site-5 | GCAGTCTATACTTTTTC | 40 | 120 |
| | TAC (SEQ ID NO: 158) | | |
| HBG1 | CTTGACCAATAGCCTTG | 160 | 260 |
| | ACA (SEQ ID NO: 159) | | |
| HBG2 | ATATTTGCATTGAGATA | 120 | 220 |
| | GTG (SEQ ID NO: 160) | | |
| AKAP9 | GAAAATAGTTGAAGAAA | 200 | 300 |
| | AAG (SEQ ID NO: 161) | | |
| PSEN1 | CACAGAAGATACCGAGA | 120 | 200 |
| | CTG (SEQ ID NO: 162) | | |

TABLE 18
List of primers used in this example to amplify off-target sites.

| Primer | Forward Sequence (5′→3′) | Reverse Sequence (5′→3′) |
|---|---|---|
| XMAS-OT1 | CAGCATTATCCATTTGCTGCCA (SEQ ID | TGGAGACAGCGAGTCTACAGC (SEQ ID |
| | NO: 163) | NO: 164) |
| XMAS-OT2 | TAACACCATTATAGCTGAAGTGGGG | TGAGTTACACACAAGCCAGTTAAATTC |
| | (SEQ ID NO: 165) | (SEQ ID NO: 166) |
| XMAS-OT3 | AGGGAGTGGACATGAGGCGA (SEQ ID | CCCAAGAGGAAGTCCCAAGG (SEQ ID |
| | NO: 167) | NO: 168) |
| Site-1-OT1 | CCTTGGGAAGAGAAGGGGTC (SEQ ID | GAGATACCGGAAGCTTTGATGTAAGA |
| | NO: 169) | (SEQ ID NO: 170) |
| Site-1-OT2 | CTTGGGGAGAAAGGTCCAGG (SEQ ID | CAAGCTTTTCCTCCTGGGATGTAAAA |
| | NO: 171) | (SEQ ID NO: 172) |
| Site-1-OT3 | CTGGCAAGCTGTTCTCACATG (SEQ ID | GAGGCTGAGGCAGGAGTATG (SEQ ID |
| | NO: 173) | NO: 174) |
| Site-3-OT1 | GTTTTCAGTAGAAGAGTATATAATACATA | ATATTCTCAGCCTAGGCCTG (SEQ ID |
| | AT (SEQ ID NO: 175) | NO: 176) |
| Site-3-OT2 | TGTTGGACATGGGTGCCTTATT (SEQ ID | TTCACCCTCTCTGGATGGCG (SEQ ID |
| | NO: 177) | NO: 178) |
| Site-3-OT3 | GCAGGAGGAGGCAGTGAAAG (SEQ ID | CAGAGAAATAACACTCTGGCAGCTG |
| | NO: 179) | (SEQ ID NO: 180) |
| Site-4-OT1 | CAGCATTTATCACGCAGTATTGTTATTG | TCATTTCGTGTTGTGCTTTATCACTTAAA |
| | (SEQ ID NO: 181) | A (SEQ ID NO: 182) |
| Site-4-OT2 | GTGAGCAGTAAACTTAATTGTTGATACA | CTTTTAGAATGAAAGTGTGCATCTTAGTA |
| | ATAAATC (SEQ ID NO: 183) | AAGAAA (SEQ ID NO: 184) |
| Site-4-OT3 | GTTCCTCACTGATTCTCAGCAGG (SEQ | CACAAAAGGGATAAATGCTCTATCCATTT |
| | ID NO: 185) | (SEQ ID NO: 186) |

We claim:

1. A method for selecting a base edited cell, the method comprising
(a) introducing into a cell a first nucleic acid sequence encoding one or more reporter proteins, a second nucleic acid sequence encoding a first sgRNA adjacent to a protospacer adjacent motif (PAM), wherein the first sgRNA comprises a protospacer sequence and is complementary to a portion of the nucleic acid sequence encoding one or more reporter proteins; a third nucleic acid encoding a second sgRNA adjacent to a protospacer adjacent motif (PAM), wherein the second sgRNA comprises a protospacer adjacent sequence and is complementary to a portion of a gene of interest to be base edited; and a fourth nucleic acid sequence encoding a base editor, wherein the first nucleic acid includes a PAM site adjacent to a base that when edited causes a change in a function or characteristic of the one or more reporter proteins and wherein the change in function or characteristic results in a detectable signal;
(b) culturing the cell of step (a) for about 48 hours to about 72 hours under conditions sufficient for expression of proteins encoded by the first, second, third and fourth nucleic acid sequences;
(e) sorting cells based on the presence or absence of a detectable signal, wherein a change in the detectable signal indicates that the base editor caused a base-to-base conversion or other genetic modification in the first nucleic acid sequence; and
(f) selecting cells exhibiting the changed detectable signal from the sorted cells, thereby selecting base edited cells.

2. The method of claim 1, wherein the base editor is selected from a cytidine deaminase base editor, an adenine base editor, Cas9-mediated adenosine base editor, and a prime editor.

3. The method of claim 1, wherein one or more of the first, second, third, and fourth nucleic acids is provided in a vector.

4. The method of claim 3, wherein the vector is an episomal vector.

5. The method of claim 1, wherein the reporter protein is a fluorescent protein.

6. The method of claim 5, wherein the fluorescent protein is a green fluorescent protein (GFP), a blue fluorescent protein (BFP), red fluorescent protein (RFP), luciferase, mCherry, or a variant or combination thereof.

7. The method of claim 6, wherein the fluorescent protein is a BFP variant comprising a histidine at amino acid position 66 (numbered relative to SEQ ID NO:1) or a fusion protein of two fluorescent proteins linked via a linker including at least one stop codon and a PAM site.

8. The method of claim 1, wherein the cell is a human cell.

9. The method of claim 8, wherein the human cell is a human pluripotent stem cell.

10. The method of claim 9, wherein the human pluripotent stem cell is a human induced pluripotent stem cell obtained from a somatic cell of a human subject having a disease-associated single nucleotide polymorphism.

11. The method of claim 1, wherein the selecting is performed using a fluorescence activated cell sorter (FACS).
